(19) United States
(12) Patent Application Publication
(10) Pub. No.: US 2007/0293937 A1
(43) Pub. Date: Dec. 20, 2007

(54) Endoluminal medical device for local delivery of cathepsin inhibitors, method of making and treating

(75) Inventors: David P. Biggs, Bloomington, IN (US); David D. Grewe, West Lafayette, IN (US); Neal E. Fearnot, West Lafayette, IN (US); Patrick H. Ruane, Redwood City, CA (US); Anthony O. Ragheb, West Lafayette, IN (US)

Correspondence Address: BRINKS HOFER GILSON & LIONE/CHICAGO/COOK, PO BOX 10395, CHICAGO, IL, 60610, US

(73) Assignee: MED Institute, Inc.

(21) Appl. No.: 11/809,779

(22) Filed: Jun. 1, 2007

Related U.S. Application Data
(63) Continuation-in-part of application No. PCT/US06/48865, filed on Dec. 22, 2006.
(60) Provisional application No. 60/755,961, filed on Jan. 3, 2006.

(51) Int. Cl. A61F 2/06; A61K 38/00; A61P 9/00

(52) U.S. Cl. 623/1.13; 514/12; 623/1.42

(57) ABSTRACT

An endoluminal medical device comprises a drug release system that releases a cathepsin inhibitor at a predetermined location within a lumen of a patient. The endoluminal devices and methods of treatment of disease can treat illnesses such as aneurysms and aortic dissections.

### RELATED APPLICATIONS

The present patent document is a continuation-in-part of PCT Application Serial No. PCT/US2006/048865, filed Dec. 22, 2006, designating the United States and which will be published in English, which claims the benefit of the filing date under 35 U.S.C. § 119(e) of Provisional U.S. Patent Application Ser. No. 60/755,961 filed Jan. 3, 2006. All of the foregoing applications are hereby incorporated by reference.

### BACKGROUND

1. Technical Field

This invention relates generally to methods and medical devices and, more particularly, to medical devices incorporating drugs, bioactive agents, therapeutic agents or diagnostic agents for treating an aneurysm and an aortic dissection. The invention also relates to kits and to treating an aorta wall adjacent to an aortic aneurysm as a preventive measure.

2. Background of the Invention

Endovascular disease may be characterized by weakened vessels due to elastin breakdown, which results in dilation of vessels and aneurysm. An aneurysm is a sac formed by localized dilatation of the wall of an artery, a vein, or the heart. Common areas where aneurysms occur and cause adverse medical conditions include the coronary arteries, the carotid arteries, various cerebral arteries, and the thoracic and abdominal aorta as well as iliac and femoral arteries. When a local dilatation of a vessel occurs, irregular blood flow patterns result in the lumen of the vessel, typically leading to clot formation. Typically, the wall of the vessel also progressively dilates and weakens, often resulting in vessel rupture. Vessel rupture, in turn, often causes dramatic negative consequences such as a stroke, when a cerebral vessel ruptures, or even death, when an abdominal aortic aneurysm (“AAA”) ruptures. In light of these consequences, improved treatment methods and devices for aneurysms are constantly being sought. Although the following discussion focuses on MA treatment, it is equally applicable to endovascular disease in other locations as well as to aortic dissections.

Aortic dissections occur when the inner layer of the aorta's artery wall splits open (dissects). The normal aorta contains collagen, elastin, and smooth muscle cells that contribute to the intima, media, and adventitia, which are the layers of the aorta. Hypertension with aging is believed to contribute to degenerative changes that may lead to breakdown of the collagen, elastin, and smooth muscle cells and, ultimately, dissection of the aorta. Aortic dissection is more likely to occur where pressure on the artery wall from blood flow is high, such as the proximal aorta or the ascending aorta (the first segment of the aorta). When the aortic wall splits, the pulses of blood can penetrate the artery wall and its inner layer, causing the aorta to tear or split further. This tear usually continues distally (away from the heart) down the descending aorta and into its major branches. Less often, the tear may run proximally (back toward the heart). Aortic dissection can also start in the descending (distal) segment of the aorta.

It is known to treat a variety of medical conditions, including endovascular disease, by temporarily or permanently introducing a medical device, and, in particular, a medical device partly or completely into the esophagus, trachea, colon, biliary tract, urinary tract, vascular system or other location within a human or veterinary patient. Many treatments of the vascular or other systems entail the introduction of a device such as a stent, a catheter, a balloon, a wire guide, a cannula or the like. For this purpose, a stent may most simply be considered as a cylinder of relatively short length which opens a body passage or lumen or which maintains a body passage or lumen in an open condition. In addition, balloons such as angioplasty or dilation balloons are expanded to open a body passage or vessel lumen, thereby causing potential trauma or injury to the expanded passage or vessel.

Endovascular grafts have been investigated as another example of a method for the treatment of aneurysmal aortic disease. One of the main concerns regarding endovascular grafting is the continued blood flow into the aneurysm after grafting which blood flow is termed in the art as an endoleak (White, et al., J. Endovasc. Surg., 3:124-125 (1996)). Endoleaks have been reported in from about 7 to about 37% of endovascular aortic aneurysm repairs with some reports placing this number as high as 44% (Marty, et al., “Endoleak After Endovascular Graft Repair of Experimental Aortic Aneurysms: Does Coil Embolization with Angiographic “Seal” Lower Intraaneursymal Pressure”, J. Vasc. Surg., 27(3):454-462 (1998)).

Specifically, endovascular grafting requires catheter placement of an endovascular prosthesis, for example, at the aortic aneurysm site. Endoleaks arising after such grafting may be caused by incomplete sealing between the endovascular prosthesis and the aortic wall or by defects within the endovascular prosthesis. In addition, back bleeding from patent lumbar and inferior mesenteric arteries following placement of the endovascular prostheses in the aorta has also been recited as a potential cause of endoleaks (Hopkinson, et al., “Current Critical Problems, New Horizons and Techniques in Vascular and Endovascular Surgery” JPIII 4.1-4.2, Presented at the 6^{th} Annual Symposium on Current Issues and New Techniques in Interventional Radiology at New York, N.Y. in November, 1998). There is uniform agreement that large endoleaks that lead to aneurysm enlargement necessitate treatment in order to prevent aneurysm rupture. It is also reported that the size of the endoleak does not appear to be a relevant factor for pressure transmission into the aneurysm (Marty, et al., “Endoleak After Endovascular Graft Repair of Experimental Aortic Aneurysms: Does Coil Embolization with Angiographic “Seal” Lower Intraaneursymal Pressure”, J. Vasc. Surg., 27(3):454-462 (1998)).

The present invention seeks to provide an improved endoluminal medical device.

### SUMMARY

According to an aspect of the present invention, there is provided an endoluminal medical device as specified in claim 1 or 24.

The preferred embodiments described herein can provide devices and methods able to stop and/or reverse the progression of endovascular disease preventing further weakening and dilation of the vessel wall.

One embodiment provides an endoluminal medical device comprising a drug release system that releases a cathepsin inhibitor at a predetermined location within a lumen of a patient. The cathepsin inhibitor may be selected from a group consisting of cysteine proteinase inhibitors, aspartic proteinase inhibitors, and serine proteinase inhibitors. The cathepsin inhibitor may be an inhibitor of cathepsin B, inhibitor of cathepsin C, inhibitor of cathepsin H, inhibitor of cathepsin L, inhibitor of cathepsin S, inhibitor of cathepsin S, inhibitor of cathepsin K, inhibitor of cathepsin O, inhibitor of cathepsin D, inhibitor of cathepsin E, inhibitor of cathepsin G, or inhibitor of cathepsin A. The cathepsin inhibitor may be selected from the group consisting of compounds CP-1, CP-2, CP-3 from Aspergillus sp.; epoxysuccinamide derivative; peptide derivative; epoxysuccinamide derivative; thiomethylene-containing aldehyde; Monobactam derivative; peptidic oxadiazole and oxathiazole derivatives; 3,4-disubstituted azetidin-2-one derivatives; 4-substituted-3-(2-amino-2-cycloalkylmethylacetamido)azetidin-2-one derivatives; lactam penam and cepham derivatives; O-benzoylhydroxylaminoe dipeptides; piperidylketocarboxylic acids; benzamidoaldehyde; ketobenzamide; heterocyclic substituted benzamide; substituted oxodiazole derivatives; ketoamide derivatives; Quinolone-containing ketoamide; dipeptide nitrile derivatives; thiadiazole derivatives; substituted benzamides; N-carbonylalkyl-benzamide; heterocyclically-substituted amide derivatives; N-cyanomethyl-amide derivative; amide derivatives; 3-acetamidoazetidin-2-one derivatives; dipeptide derivatives; cyclic amide hypercalcaemia and dipeptide derivative; substituted pyrrolidin-2-one derivative; N-aminoalkyl-N-hydrazine derivatives; diacyl carbohydrazine compounds; thiazole guanidine derivatives; morpholinoethoxybenzofuran compounds; butyl amide derivatives; sulfonamide and carboxamide derivatives; modulated amyloid precursor protein and tau protein; hydroxypropylamide peptidomimetics; hydroxystatine amide hydroxyphosphonate peptidomimetics; hydroxyamino acid amide derivatives; peptoid compounds; heteroaryl amidines methylamidines and guanidines; 1,2,5-thiadiazolidin-3-one 1,1-dioxide derivatives; transhexahydro-pyrrolo[3,2-b]pyrrolone derivatives; pyrolopyrrolidine derivatives; furopyrrolidine derivatives; and anthraquinone derivatives; and mixtures thereof. The cysteine proteinase inhibitor may be an endogenous cathepsin inhibitor. The cysteine proteinase inhibitor may be an exogenous cysteine proteinase inhibitor, wherein the exogenous cysteine proteinase inhibitor is a small peptide derivative or a beta phosphonic acid. The cathepsin inhibitor may be a dipeptide nitrile. The drug release system may be a stent, a stent graft or other suitable prosthesis. The drug release system may further comprise a tubular graft material supported by the stent. The stent may be a self-expanding stent or a balloon expandable stent. The graft material may comprise an extracellular matrix material, such as a small intestine submucosa. The device may further comprise a delivery system for delivering the prosthesis. The drug release system may be integrated with the delivery system. The device may also comprise a drug delivery system for delivering the device. The drug release system may be integrated with the delivery system for delivering the device. The delivery system may comprise a balloon. The drug release system may be integrated with the balloon. The balloon may include one or more perforations configured to release the cathepsin inhibitor. In another example, the cathepsin inhibitor may be carried on the outer surface of the balloon. The balloon may be a torroidal balloon and may further include a carrier. The balloon may be a photodynamic therapy balloon. The drug release system may include an expandable wire basket. The endoluminal medical device may further comprise a polymer layer to provide a controlled release of the cathepsin inhibitor at the predetermined location. The device may be for treatment of aneurysms, such as an abdominal aortic aneurysm, or aortic dissection.

Another embodiment provides an endoluminal medical device for treating an aneurysm, the device comprising a drug release system that releases a cathepsin inhibitor at a predetermined location near the aneurysm. The cathepsin inhibitor may be selected from a group consisting of cysteine proteinase inhibitor, aspartic proteinase inhibitor, and serine proteinase inhibitor. The cathepsin inhibitor may be an inhibitor of cathepsin B, inhibitor of cathepsin C, inhibitor of cathepsin H, inhibitor of cathepsin L, inhibitor of cathepsin S, inhibitor of cathepsin S, inhibitor of cathepsin K, inhibitor of cathepsin O, inhibitor of cathepsin D, inhibitor of cathepsin E, inhibitor of cathepsin G, or inhibitor of cathepsin A. The cathepsin inhibitor may be selected from the group consisting of compounds CP-1, CP-2, CP-3 from Aspergillus sp.; epoxysuccinamide derivative; peptide derivative; epoxysuccinamide derivative; thiomethylene-containing aldehyde; Monobactam derivative; peptidic oxadiazole and oxathiazole derivatives; 3,4-disubstituted azetidin-2-one derivatives; 4-substituted-3-(2-amino-2-cycloalkylmethylacetamido)azetidin-2-one derivatives; β-lactam penam and cepham derivatives; O-benzoylhydroxylaminoe dipeptides; piperidylketocarboxylic acids; benzamidoaldehyde; ketobenzamide; heterocyclic substituted benzamide; substituted oxodiazole derivatives; ketoamide derivatives; Quinolone-containing ketoamide; dipeptide nitrile derivatives; thiadiazole derivatives; substituted benzamides; N-carbonylalkyl-benzamide; heterocyclically-substituted amide derivatives; N-cyanomethyl-amide derivative; amide derivatives; 3-acetamidoazetidin-2-one derivatives; dipeptide derivatives; cyclic amide hypercalcaemia and dipeptide derivative; substituted pyrrolidin-2-one derivative; N-aminoalkyl-N-hydrazine derivatives; diacyl carbohydrazine compounds; thiazole guanidine derivatives; morpholinoethoxybenzofuran compounds; butyl amide derivatives; sulfonamide and carboxamide derivatives; modulated amyloid precursor protein and tau protein; hydroxypropylamide peptidomimetics; hydroxystatine amide hydroxyphosphonate peptidomimetics; hydroxyamino acid amide derivatives; peptoid compounds; heteroaryl amidines methylamidines and guanidines; 1,2,5-thiadiazolidin-3-one 1,1-dioxide derivatives; transhexahydro-pyrrolo[3,2-b]pyrrolone derivatives; pyrolopyrrolidine derivatives; furopyrrolidine derivatives; and anthraquinone derivatives; and mixtures thereof. The cysteine proteinase inhibitor may be an endogenous cathepsin inhibitor. The cysteine proteinase inhibitor may be an exogenous cysteine proteinase inhibitor, wherein the exogenous cysteine proteinase inhibitor is a small peptide derivative or a beta phosphonic acid. The cathepsin inhibitor may be a dipeptide nitrile. The aneurysm may be an abdominal aortic aneurysm.

In yet another embodiment, there is provided a method of making an endoluminal medical device, comprising providing a drug release system as part of the medical device that releases a cathepsin inhibitor at a predetermined location within a lumen of a patient. The method further includes providing a at least one polymer layer configured to provide a controlled release of the cathepsin inhibitor. The cathepsin inhibitor may be coated on the drug release system.

In yet another embodiment, there is provided a method for treating an aneurysm, the method comprising delivering a cathepsin inhibitor releasing device. The releasing device may be an endoluminal medical device comprising a drug release system releasing a cathepsin inhibitor at the location near the aneurysm. The endoluminal device may be a stent graft for treating an aortic aneurysm. The cathepsin inhibitor may be selected from the group consisting of cysteine proteinase inhibitors, aspartic proteinase inhibitors, and serine proteinase inhibitors. The cathepsin inhibitor may be an inhibitor of cathepsin B, inhibitor of cathepsin C, inhibitor of cathepsin H, inhibitor of cathepsin L, inhibitor of cathepsin S, inhibitor of cathepsin S, inhibitor of cathepsin K, inhibitor of cathepsin O, inhibitor of cathepsin D, inhibitor of cathepsin E, inhibitor of cathepsin G, or inhibitor of cathepsin A. The cathepsin inhibitor may be selected from the group consisting of compounds CP-1, CP-2, CP-3 from Aspergillus sp.; epoxysuccinamide derivative; peptide derivative; epoxysuccinamide derivative; thiomethylene-containing aldehyde; Monobactam derivative; peptidic oxadiazole and oxathiazole derivatives; 3,4-disubstituted azetidin-2-one derivatives; 4-substituted-3-(2-amino-2-cycloalkylmethylacetamido)azetidin-2-one derivatives; lactam penam and cepham derivatives; O-benzoylhydroxylaminoe dipeptides; piperidylketocarboxylic acids; benzamidoaldehyde; ketobenzamide; heterocyclic substituted benzamide; substituted oxodiazole derivatives; ketoamide derivatives; Quinolone-containing ketoamide; dipeptide nitrile derivatives; thiadiazole derivatives; substituted benzamides; N-carbonylalkyl-benzamide; heterocyclically-substituted amide derivatives; N-cyanomethyl-amide derivative; amide derivatives; 3-acetamidoazetidin-2-one derivatives; dipeptide derivatives; cyclic amide hypercalcaemia and dipeptide derivative; substituted pyrrolidin-2-one derivative; N-aminoalkyl-N-hydrazine derivatives; diacyl carbohydrazine compounds; thiazole guanidine derivatives; morpholinoethoxybenzofuran compounds; butyl amide derivatives; sulfonamide and carboxamide derivatives; modulated amyloid precursor protein and tau protein; hydroxypropylamide peptidomimetics; hydroxystatine amide hydroxyphosphonate peptidomimetics; hydroxyamino acid amide derivatives; peptoid compounds; heteroaryl amidines methylamidines and guanidines; 1,2,5-thiadiazolidin-3-one 1,1-dioxide derivatives; transhexahydro-pyrrolo[3,2-b]pyrrolone derivatives; pyrolopyrrolidine derivatives; furopyrrolidine derivatives; and anthraquinone derivatives; and mixtures thereof. The cysteine proteinase inhibitor may be an endogenous cathepsin inhibitor. The cysteine proteinase inhibitor may be an exogenous cysteine proteinase inhibitor, wherein the exogenous cysteine proteinase inhibitor is a small peptide derivative or a beta phosphonic acid. The cathepsin inhibitor may be a dipeptide nitrile.

### BRIEF DESCRIPTION OF THE DRAWINGS

Embodiments of the present invention are described below, by way of example only, with reference to the accompanying drawings, in which:

FIG. 1 is a cross-sectional view of a first preferred embodiment of the present invention;

FIG. 2 is a cross-sectional view of another preferred embodiment of the present invention;

FIG. 3 is a cross-sectional view of yet another preferred embodiment of the present invention;

FIG. 4 is a cross-sectional view of a further preferred embodiment of the present invention;

FIG. 5 is a cross-sectional view of an additional preferred embodiment of the present invention;

FIG. 6 is a cross-sectional view of an additional preferred embodiment of the present invention;

FIGS. 7A and 7B are cross-sectional views of an additional preferred embodiment of the present invention;

FIG. 8 is a partial, enlarged top view of the embodiment shown in FIG. 6;

FIG. 9 is an enlarged, sectional view along lines 9-9 of FIG. 8;

FIGS. 10A-10D are enlarged cross-sectional views along lines 10-10 of FIG. 8;

FIG. 11 shows a modular bifurcated aortic endoluminal medical device with graft material impregnated with a cathepsin inhibitor, implanted within an aneurysmal aorta;

FIG. 12 shows a stent graft impregnated with a cathepsin inhibitor;

FIG. 13 illustrates a catheter-based aneurysmal drug release system of the present invention;

FIG. 14 illustrates an additional embodiment of catheter-based aneurysmal drug release systems of the present invention;

FIG. 15 illustrates yet another embodiment of catheter-based aneurysmal drug release systems of the present invention;

FIG. 16 illustrates yet another embodiment of catheter-based aneurysmal drug release systems of the present invention;

FIG. 17 illustrates an additional embodiment of catheter-based aneurysmal drug release systems of the present invention;

FIG. 18 illustrates yet another embodiment of catheter-based aneurysmal drug release systems of the present invention;

FIG. 19 is a sectional view of a balloon and balloon catheter inserted into an artery of the patient with the balloon inflated to cause the microencapsulated spheres to become embedded in the wall of the artery; and

FIG. 20 is a cross-sectional view of an additional preferred embodiment of the present invention.

### DETAILED DESCRIPTION OF THE DRAWINGS AND THE PRESENTLY PREFERRED EMBODIMENTS

The following description discloses endoluminal medical devices, which release cathepsin inhibitors, and methods of using these endoluminal medical devices to prevent breakdown of host connective tissue and treat variety of other diseases and conditions, including endovascular disease. By including cathepsin inhibitors with the device, the progression of local endovascular disease may be stopped and/or reversed, preventing further weakening and dilation of vessel wall. These types of devices may preferably be used for treatment of aneurysms, especially aortic abdominal aneurysms.

I. Definition of Terms

Unless defined otherwise, all technical and scientific terms used herein have the same meaning as commonly understood to one of ordinary skill in the art to which this invention belongs.

The term “cathepsin inhibitor” refers to a compound or a plurality of compounds, chemical compositions, polypeptides, polynucleotides, etc. capable of interfering with, such as downregulating, suppressing or neutralizing enzymatic activity of cathepsin family members. In accordance with the present invention the inhibitor preferably interacts with its ligand, for example, by specifically binding to said ligand. Cathepsin inhibitor can be in the form of a pharmaceutically acceptable salt, free base, solvate, hydrate, stereoisomer, clathrate or prodrug thereof. Inhibitory activity of cathepsin inhibitor may be determined by an assay or animal model well-known in the art. Cathepsin inhibitor may include any inhibitor of cysteine protease cathepsins, such as for example, cathepsin B, C, H, K, L, O, and S; aspartic protease cathepsins, such as, for example, cathepsin D and E; and serine protease cathepsins, such as, for example, cathepsin A and G. Examples of cathepsin inhibitors are provided below.

“Specifically binding” means “specifically interacting with” whereby the interaction may be covalent, non-covalent, hydrogen bond, electrostatic and/or van der Waals. Thus, an inhibitor may be an antagonist or a compound, which inhibits or decreases, for example, the interaction between a protein and another molecule. Examples of such inhibitors are described below in more detail and may be obtained by the methods described herein.

The term “pharmaceutically acceptable salt thereof” includes an acid addition salt or a base salt.

As used herein and unless otherwise indicated, the term “prodrug” means a cathepsin inhibitor derivative that can hydrolyze, oxidize, or otherwise react under biological conditions (in vitro or in vivo) to provide an active compound, particularly a cathepsin inhibitor. Examples of prodrugs include, but are not limited to, derivatives and metabolites of a cathepsin inhibitor that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

The term “pharmaceutically acceptable carrier” includes any material which, when combined with a cathepsin inhibitor, allows the inhibitor to retain biological activity, such as the ability to inhibit cathepsins and breakdown of host connective tissue, and is non-reactive with the subject's immune system. Examples include, but are not limited to, any of the standard pharmaceutical carriers such as a phosphate buffered saline solution, water, emulsions such as oil/water emulsions, various polymer carrier materials, and various types of wetting agents. Compositions comprising such carriers are formulated by well known conventional methods (see, for example, Remington's Pharmaceutical Sciences, Chapter 43, 14th Ed., Mack Publishing Co., Easton, Pa.).

The term “polynucleotide” refers to a linear or circular sequence of nucleotides. The nucleotides may be in a linear or circular sequence of polyribonucleotides or polydeoxyribonucleotides, or a mixture of both. Examples of polynucleotides in the context of the present invention include single- and double-stranded DNA, single- and double-stranded RNA, and hybrid molecules that have both mixtures of single- and double-stranded DNA and RNA. Further, the polynucleotides of the present invention may have one or more modified nucleotides.

As used herein, the phrase “controlled release” refers to the release of a therapeutic agent at a predetermined rate. A controlled release may be characterized by a drug elution profile, which shows the measured rate that the material is removed from a material-coated device in a given solvent environment as a function of time. A controlled release does not preclude an initial burst release associated with the deployment of the medical device, because in some embodiments of the invention an initial burst, followed by a more gradual subsequent release, may be desirable. The release may be a gradient release in which the concentration of the therapeutic agent released varies over time or a steady state release in which the therapeutic agent is released in equal amounts over a certain period of time (with or without an initial burst release).

The term “graft” means any replacement for a bodily tissue or a function of the bodily tissue. A graft may be of any of the known types and may also be of a type which may be transplanted from a donor to a recipient to repair a part of a body, and in some cases the patient can be both donor and recipient. For example, a graft may replace tissue that has been destroyed or create new tissue where none exists.

The term “tubular” refers to the general shape of an endoluminal device which allows the module to carry fluid along a distance or fit within a tubular structure such as an artery. Tubular prosthetic modules include both branched and bifurcated modules.

The term “stent” is intended to have a broad meaning and encompasses any expandable prosthetic device for implantation in a body passageway (e.g., a lumen or artery). A stent may be used to obtain and maintain the patency of the body passageway while maintaining the integrity of the passageway. A stent may also be used to form a seal.

The term “stent graft” refers to a type of endoluminal prosthesis made of a tubular graft material and supported by at least one stent.

As used in this specification, the term “body passageway” is intended to have a broad meaning and encompasses any duct (e.g., natural or iatrogenic) within the human body and can include a member selected from the group comprising: blood vessels, respiratory ducts, gastrointestinal ducts, and the like.

The term “healing” means replacing, repairing, healing, or treating of damaged or diseased tissues of a patient's body.

The terms “patient,” “subject,” and “recipient” as used in this application refer to any mammal, especially humans.

Researchers have hypothesized that the development, expansion and rupture of AAAs are related to connective tissue destruction. For a discussion of this hypothesis, see for example, “Pharmacologic suppression of experimental abdominal aortic aneurysms: A comparison of doxycycline and four chemically modified tetracyclines,” Curci, John A., Petrinec, Drazen, et al., Journal of Vascular Surgery, December 1998, vol. 28, no. 6, 1082-1093 (hereinafter “Curci article. Connective tissue destruction, in turn, has been linked to the presence of a number of enzymes which break down blood vessel wall connective tissues such as elastin. Examples of such “elastolytic” enzymes include serine proteinases, such as cathepsins. It has been found that increased levels of some elastolytic enzymes are typically present in AAAs.

Cathepsins may be classified as cysteine protease cathepsins, aspartic protease cathepsins, and serine protease cathepsins.

Cysteine proteases, for example, belong to the enzyme classification EC 3.4.22 (Barrett, A. J., N. D. Rawlings, et al. Handbook of proteolytic enzymes. London, Academic Press), and include proteases such as cathepsins B, C (also known as dipeptidyl peptidase I or DPPI), H, K, L, O and S. (A. J. Barrett et al., Perspectives in Drug Discovery and Design, 6:1 (1996); Pagano M B, et al., PNAS, 104(8):2855-2860 (February 2007)).

Aspartic protease cathepsins include, for example, cathepsins D and E.

Serine protease cathepsins include, for example, cathepsin G and A.

Cathepsins are involved in the normal proteolysis and turnover of target proteins and tissues as well as in initiating proteolytic cascades by proenzyme activation and in participating in MHC class II molecule expression (Baldwin, Proc. Natl. Acad. Sci., 90: 6796-6800 (1993); Mixuochi, Immunol. Lett., 43:189-193 (1994)). As previously discussed, in addition to playing a role in lysosomal, endosomal, and extracellular protein degradation, cathepsins have also been implicated in many disease processes. Accordingly, it may be desirable to inhibit cathepsins at a location in a body to stop and/ore prevent further progression and/or development of a vascular disease, such as AAA or aortic dissection.

II. Medical Devices Containing Cathepsin Inhibitors

One aspect of the present invention provides an endoluminal medical device (“medical device”) comprising a drug release system that releases a cathepsin inhibitor at a predetermined location within a lumen of a patient. One or more cathepsin inhibitors may be provided for release from the medical device.

The cathepsin inhibitors may be included, for example, as part of at least a portion of the base material forming the drug release system of the medical device itself; be contained within reservoir, a well or a groove; be within a carrier material deposited on at least a portion of the medical device, or as a separate layer deposited on at least a portion of the medical device (the layer may optionally be over coated with another layer) or on at least a portion of the medical device that has been coated with a primer layer for increased adhesion; or within the hollow walls of the device; or any combination of these. The cathepsin inhibitor may also be included in a separate carrier layer (or a multi-layered structure) that may be placed between the elements of the medical device. For example, the separate layer may be placed between a stent and a graft material.

The inventors have determined that there are several approaches to controlling the release of a cathepsin inhibitor from a medical device, which will be described in more detail below. The controlled release allows for smaller amounts of the cathepsin inhibitor to be released for longer periods of time (days, weeks, years), preferably in a zero order elution profile manner.

A. Cathepsin Inhibitors

Either a single cathepsin inhibitor compound or a combination of cathepsin inhibitor compounds may be used. Consequently, as used herein and in the appended claims, and as noted above, the term “cathepsin inhibitor” refers to either a single compound or a combination of compounds that inhibit cathepsin. Cathepsin inhibitor compositions described below, including carrier materials, may also be used.

Cathepsin inhibitors may be classified as cysteine proteinase inhibitors, aspartic proteinase inhibitors, or serine proteinase inhibitors. For a comprehensive review of cathepsin inhibitors see Kim W. and Kang K, “Recent developments of cathepsin inhibitors and their selectivity,” Expert Opin. Ther. Patents (2002) 12(3), pp 419-432.

1) Cysteine Proteinase Inhibitors

Cathepsin inhibitor may be, for example, a cysteine proteinase inhibitor. Cysteine proteinase inhibitors include endogenous inhibitors, cystatins, which are divided into three families. Members of family I, or stefins, are small proteins about 100 amino acids, which have no internal disulfide bonds. The inhibitors of family II contain two disulfides and have about 120 amino acids. The members of family III are the larger glycoproteins, called kininogens.

Family I of cystatins includes, for example cystatin A, which consists of 98 amino acids. The inhibitory activity of cystatin A towards papain-like proteinase is believed to be due to the interaction of the wedge-shaped edge of the inhibitor with the active site cleft of the enzyme (Estrada S. et al. Biochemistry (1999) 38:7339-7345; Pavlova A. et al. FEBS Lett. (2000) 487(2):156-160). The inhibitory wedge is formed by three segments of the protein, N-terminal end of the chain and two hairpin loops, one central and one closer to the C-terminus.

Cysteine protease inhibitors may include, but are not limited to inhibitors of cathepsins B, C, H, L, S, K, and O.

Exemplary cysteine proteinase inhibitors include, but are not limited to, inhibitors of cathepsins B and L, including inhibitors shown in Table A below.

TABLE A

| Com- | | |
|---|---|---|
| pound | Name | Structure |
| 1 | CP-1, CP-2, CP-3 from Aspergillus sp. | 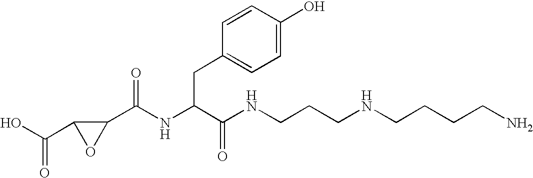 |
| 2 | epoxysuccinamide derivative | 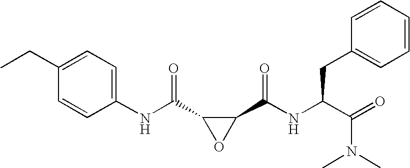 |
| 3 | peptide derivative | 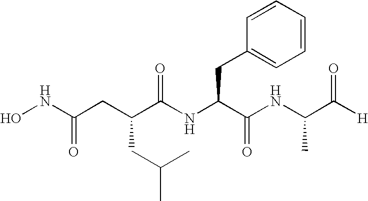 |
| 4 | epoxysuccinamide derivative | 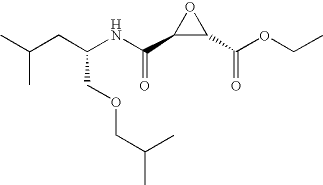 |
| 5 | thiomethylene-containing aldehydes | 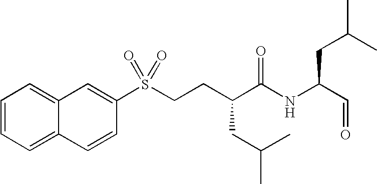 |
| 6 | Monobactam derivatives | 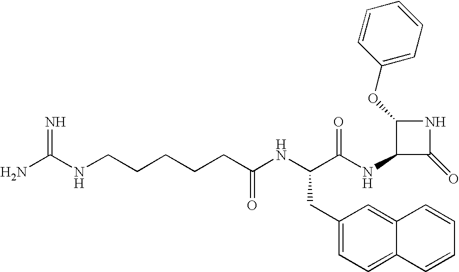 |
| 7 | peptidic oxadiazole and oxathiazole derivatives | 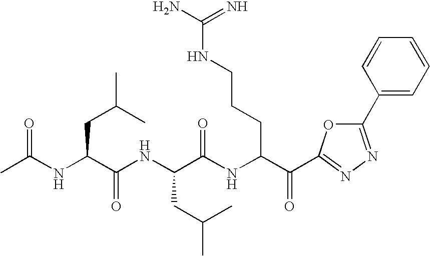 |
| 8 | 3,4-disubstituted azetidin-2-one derivatives | 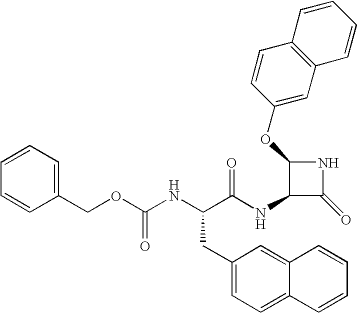 |
| 9 | 4,substituted-3-(2-amino-2- cycloalkylmethylacetamido)azetidin- 2-one derivatives | 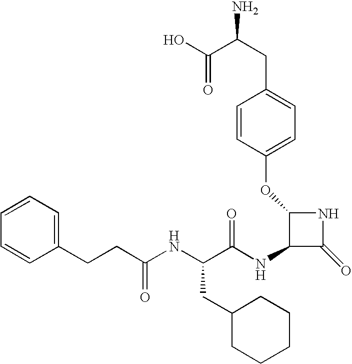 |
| 10 | β-lactam penam and cepham derivatives | 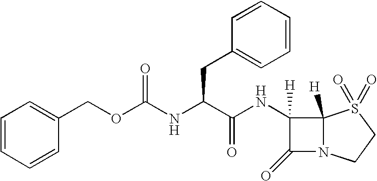 |
| 11 | O-benzoylhydroxylaminoe dipeptides | 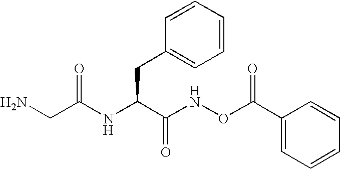 |
| 12 | piperidylketocarboxylic acids | 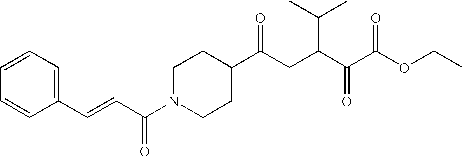 |
| 13 | benzamidoaldehyde | 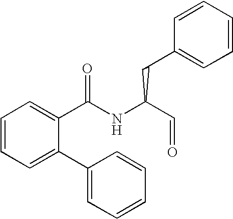 |
| 14 | ketobenzamide | 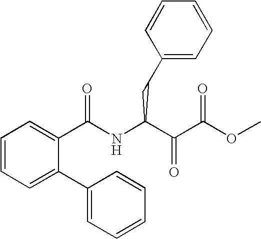 |
| 15 | hetercyclic substituted benzamide | 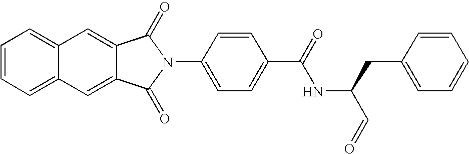 |
| 16 | substituted oxodiazole derivatives | 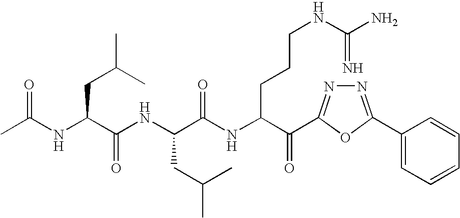 |
| 17 | ketoamide derivatives | 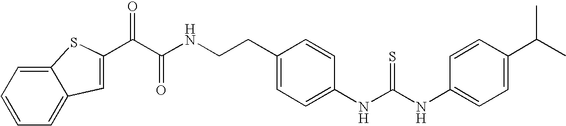 |
| 18 | Quinolone-containing ketoamide | 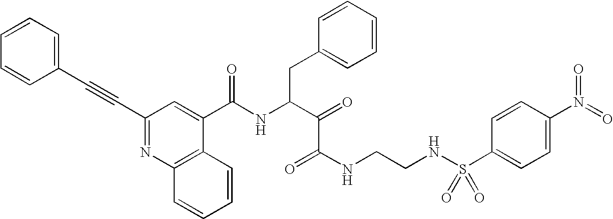 |
| 19 | dipeptide nitrile derivatives | 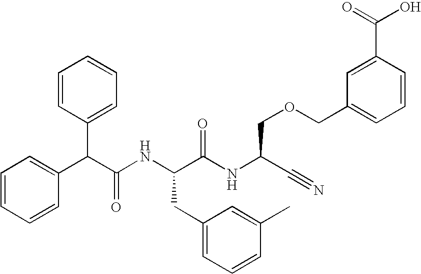 |
| 20 | thiadiazole derivatives | 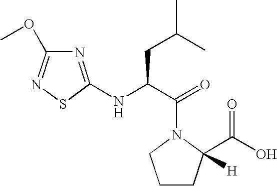 |
| 21, 25 | substituted benzamides | 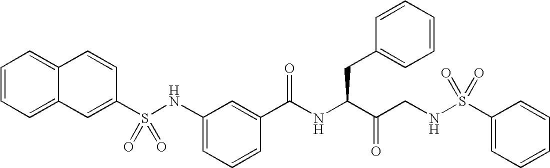 |
| | | 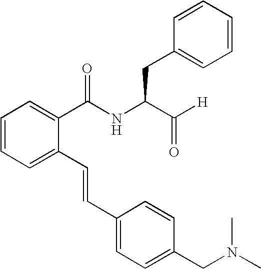 |
| 22 | N-carbonylalkyl-benzamide | 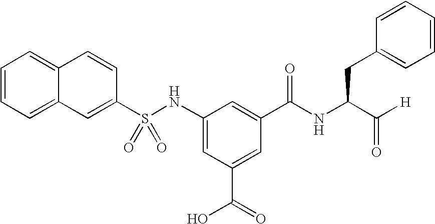 |
| 23, 24, 26 | heterocyclically-substituted amide derivatives | 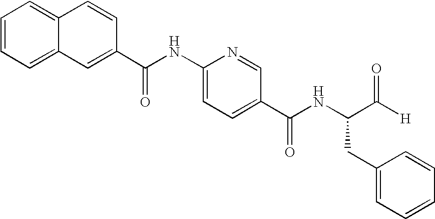 |
| | | 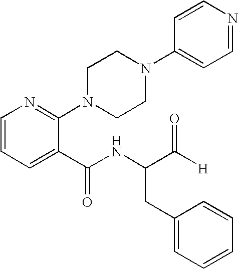 |
| | | 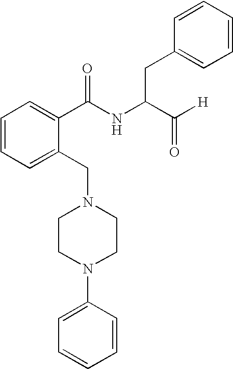 |

Other exemplary cysteine proteinase inhibitors include, but are not limited to, inhibitors of cathepsins K and S, including compounds shown in Table B below.

TABLE B

| Compound | Name | Structure |
|---|---|---|
| 28 | N-cyanomethyl-amide derivative | 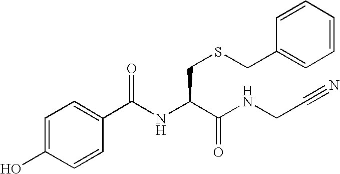 |
| 29 | amide derivatives | 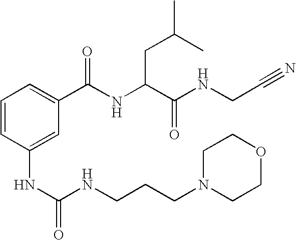 |
| 27, 30 | amide derivatives | 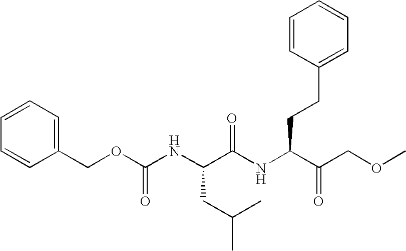 |
| | | 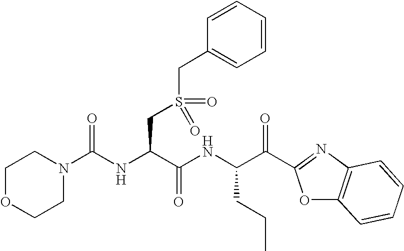 |
| 31 | 3-acetamidoazetidin-2-one derivatives | 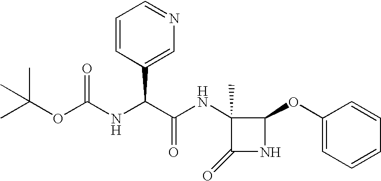 |
| 32 | dipeptide derivatives | 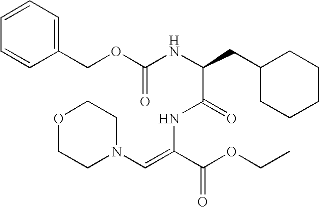 |
| 33 | cyclic amide hypercalcaemia and dipeptide derivative | 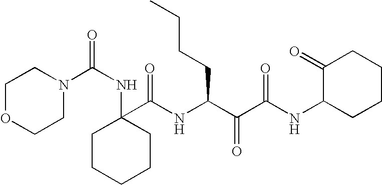 |
| 34 | substituted pyrrolidin-2-one derivative | 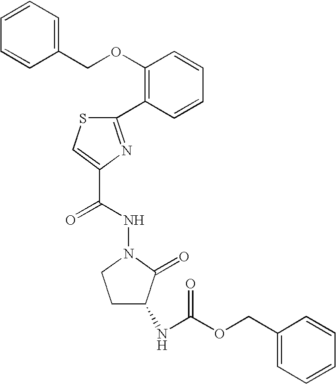 |
| 35 | N-aminoalkyl-N-hydrazine derivatives | 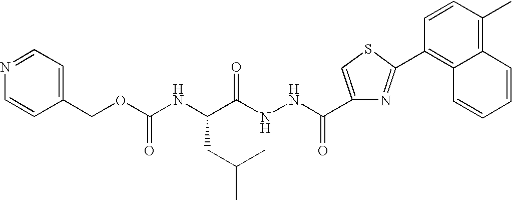 |
| 36 | diacyl carbohydrazine compounds | 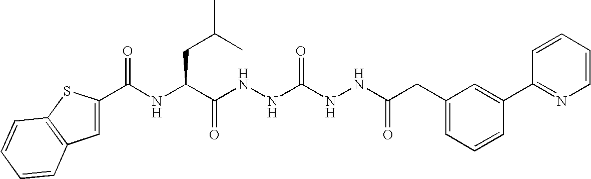 |
| 37 | thiazole guanidine derivatives | 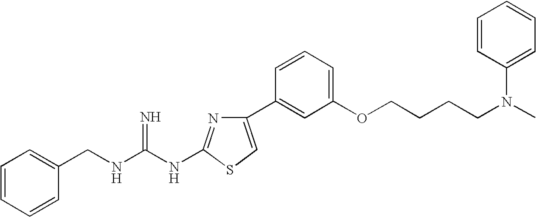 |
| 38 | mopholinoethoxybenzofuran compounds | 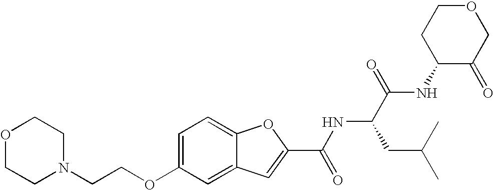 |
| 39, 40 | butyl amide derivatives | 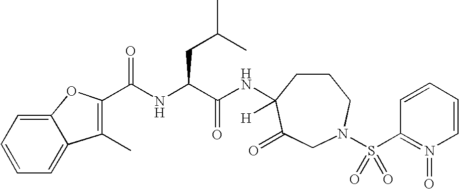 |
| | | 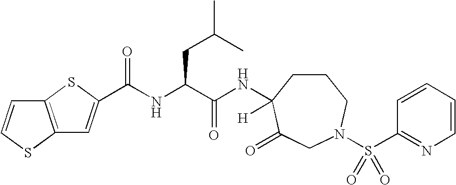 |

Additional cysteine proteinase inhibitors include, but are not limited to, inhibitors of cathepsin C. Cathepsin C inhibitors include for example, synthetic inhibitors, such as diazomethyl ketones (Gly-Phe-CHN₂; Z-Phe-Ala-CHN₂; and Z-Phe-Gly-Phe-CHN₂) reported by Green G D J and Shaw J (Green G D J, Shaw J, J. Biol. Chem., 256:1923-1028 (1981)) and diazomethyl ketones, such as GF-dmk (Gly-Phe-doazomethylketone; purchased from Enzyme Systems Products, Livermore, Calif.) reported by Bidere N, et al. (Bidere N, et al., The Journal of Biological Chemistry, 277(35):32339-32347 (2002)). One example of an effective cathepsin C inhibitor includes Gly-Phe-CHN₂ with k_{obs}/[l] of 10⁴ M⁻¹ s⁻¹ (Green G D J, Shaw J, J. Biol. Chem., 256:1923-1028 (1981)).

Additional examples of cathepsin C inhibitors include those also reported by Tompson S A, et al. (Tompson S A, et al., J. Med. Chem., 29 (1):104-111 (1986)), such as compounds of formula VI:

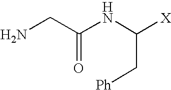

Where X is CONH₂, CSNH₂, CN, or trans-CH═CHCOOMe.

A reversible nitrile inhibitor Gly-NHCH(CH₂Ph)CN was found to inhibit cathepsin C with Kₗ value of 2.7 μM and Gly-Phe-VS—CH₃ was found to inhibit cathepsin C with k₂/Kₗ of 56 M⁻¹ s⁻¹ (Tompson S A, et al., J. Med. Chem., 29:104-111 (1986)).

Other exemplary synthetic inhibitors of cathepsin C include compounds described in U.S. Pat. No. 6,844,316 B2, which is incorporated herein by reference in its entirety. U.S. Pat. No. 6,844,316 B2 also provides methods for synthesis of these compounds. Specifically, exemplary inhibitors of cathepsin C include compounds having the general Formula VII:

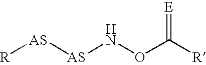

Wherein:

R is an acyl-residue including a urethane or peptide, or a branched or unbranched C₁-C₉ alkyl chain, a branched or unbranched C₂-C₉ alkenyl chain, a branched or unbranched C₂-C₉ alkynyl chain, a C₃-C₉ cycloalkyl, C₄-C₉ carbocyclic, C₅-C₁₄ aryl, C₃-C₉ heteroaryl, C₃-C₉ heterocyclic, all of the above residues optionally being substituted, or R is H, the residue AS-AS is a dipeptide or a mimetic thereof,

E is O or S, and

R′ is a branched or unbranched C₁-C₉ alkyl chain, a branched or unbranched C₂-C₉ alkenyl chain, a branched or unbranched C₂-C₉ alkynyl chain, a C₃-C₉ cycloalkyl, C₄-C₉ cycloalkenyl, C₂-C₉ heterocycloalkyl, C₃-C₉ heterocycloalkenyl, C₅-C₁₄ aryl, C₃-C₉ heteroaryl, C₃-C₉ heterocyclic, wherein the heterocycloalkyl, heterocycloalkenyl, heteroaryl, heterocyclic residue can have up to 6 hetero atoms in the ring, or R′ is an amino acid or a peptide or a mimetic thereof, all of the above residues optionally being substituted, or R′ is H or alkoxy, alkenyloxy, alkynyloxy, carbocyclicoxy, heteroraryloxy, heterocyclicoxy, thioether or a substituted residue thereof or pharmaceutically acceptable salts thereof.

Examples of amino acids are L and D-amino acids, N-methyl-amino-acids; allo- and threo-forms of Ile and Thr, which can, e.g. be α-, β- or ω-amino acids, whereof α-amino acids are preferred.

Preferably, the group AS-AS is bound with a peptide bond to R.

In one embodiment, the residue R is a phenyl or naphthyl residue that optionally is mono-, di-, or poly-substituted by C₁-C₆ alkyl, C₂-C₆ alkenyl, C₂-C₆ alkynyl, acyl, C₁-C₆ alkoxy, C₂-C₆ alkenyloxy, C₂-C₆ alkynyloxy, C₃-C₉ heteroaryloxy, C₃-C₉ heterocyclicoxy, C₁-C₆ thioether or a substituted residue thereof, NO₂, NH₂, F, Cl, Br, I atoms or groups. The above residues can be branched or unbranched.

In another embodiment, R′ may be NO₂, NH₂, F, Cl, Br, I atoms or groups or is a phenyl or naphthyl residue, which is optionally mono-, di-, or poly-substituted by C₁-C₆ alkyl, C₂-C₆ alkenyl, C₂-C₆ alkynyl, C₁-C₆ acyl, C₁-C₆ alkoxy, C₂-C₆ alkenyloxy, C₂-C₆ alkynyloxy, C₃-C₉ heteroaryloxy, C₃-C₉ heterocyclicoxy, C₁-C₆ thioether or a substituted residue thereof, NO₂, NH₂, F, Cl, Br, I atoms or groups, or

when R′ is

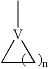

wherein V is N or CH and n=1-6 or pharmaceutically acceptable salts thereof.

In another embodiment, cathepsin C inhibitors include compounds of formula VII, wherein R′ is 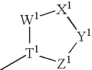
Wherein

T¹ is CH or N,

W¹, X¹, Y¹ and Z¹ are independently from each other selected from CH₂, NR², N⁺(R³)₂, O, S, SO, S(R⁴)₂, and SO₂ with the proviso that at least two or three of W¹, X¹, Y¹ and Z¹ are CH₂-groups, R², R³ and R⁴ are independently from each other a branched or unbranched C₁-C₉ alkyl chain, a branched or unbranched C₂-C₉ alkenyl chain, a branched or unbranched C₂-C₉ alkynyl chain, C₃-C₉ cycloalkyl, C₄-C₉ cycloalkenyl or H or pharmaceutically acceptable salts thereof.

In another illustrative embodiment, cathepsin C inhibitors include compounds of formula VII, wherein R′ is

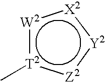

Wherein

T² is C or N⁺,

W², X², Y² and Z² are independently from each other CH, N, N⁺R⁵ or S⁺R⁶ with the proviso that at least two or three of W², X², Y² and Z² are CH₂— groups,

R⁵ and R⁶ are independently from each other a branched or unbranched C₁-C₉ alkyl chain, a branched or unbranched C₂-C₉ alkenyl chain, a branched or unbranched C₂-C₉ alkynyl chain, C₃-C₉ cycloalkyl, C₄-C₉ cycloalkenyl or H or pharmaceutically acceptable salts thereof.

Furthermore, cathepsin C inhibitors include compounds of formula VII, wherein R′ is

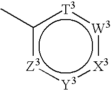

Wherein

T³, W³, X³, Y³ and Z³ independently from each other are CH, N⁺R⁷ or S⁺R⁸ with the proviso that at least two or three of T³, W³, X³, Y³ and Z³ are CH₂— groups,

R⁷ and R⁸ are independently a branched or unbranched C₁-C₉ alkyl chain, a branched or unbranched C₂-C₉ alkenyl chain, a branched or unbranched C₂-C₉ alkynyl chain, C₃-C₉ cycloalkyl, C₄-C₉ cycloalkenyl or H,

or pharmaceutically acceptable salts thereof.

Further cathepsin C inhibitors include compounds of formula VII, wherein R′ is

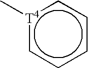

Wherein T⁴ is C or N⁺, or pharmaceutically acceptable salts thereof.

The residues R′ defined herein may be mono- or poly-substituted by, e.g., alkyl, alkoxy, alkenyl, alkynyl, acyl, carbocyclic, aryl, heteroaryl, heterocyclic, thioether, NO₂, NH₂, F, Cl, Br, I atoms or groups, mono- or di-substitution being preferred. It is especially preferred that the substituents are not substituted any further.

Cathepsin C inhibitor compounds also include those of formula VII, wherein R′ is an amino acid, a peptide or a dipeptide or a mimetic thereof.

The salts of the compounds described above, assuming that they have basic properties, be in the form of inorganic or organic salts.

The compounds may be converted into and used as acid addition salts, especially pharmaceutically acceptable acid addition salts. The pharmaceutically acceptable salt generally takes a form in which a basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toulenesulfonic, cyclohexanesulfamic, salicylic, saccharinic or trifluoroacetic acid. All pharmaceutically acceptable acid addition salt forms of the compounds described herein are also included.

In view of the close relationship between the free compounds and the compounds in the form of their salts, whenever a compound is referred to in this context, a corresponding salt is also intended, provided such is possible or appropriate under the circumstances.

Where the compounds have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms of the compounds may exist as polymorphs and as such are intended to be included in the present invention. In addition, some of the compounds may form solvates with water (i.e. hydrates) or common organic solvents, and such solvates are also intended to be encompassed within the scope of this invention.

The compounds, including their salts, can also be obtained in the form of their hydrates, or include other solvents used for their crystallization.

The term “acyl” can denote a C₁₋₂₀ acyl residue, preferably a C₁₋₈ acyl residue and especially preferred a C₁₋₄ acyl residue, “carbocyclic” or “cycloalkyl” can denote a C₃₋₁₂ carbocyclic residue, preferably a C₄, C₅ or C₆ carbocyclic residue, “cycloalkenyl” can denote a C₃₋₁₂ carbocyclic residue, preferably a C₅ or C₆ carbocyclic residue having at least one double band at any desired location. “Heteroaryl” is defined as an aryl residue, wherein 1 to 4, preferably 1, 2 or 3 ring atoms are replaced by heteroatoms like N, S or O. “Heterocycloalkyl” or “heterocyclic” is defined as a cycloalkyl residue, wherein 1, 2 or 3 ring atoms are replaced by heteroatoms like N, S or O. “Heterocycloalkenyl” is defined as a heterocycloalkyl residue having at least one double bond at any desired location. The expression “alkyl” can denote a C₁₋₅₀ alkyl group, preferably a C₆₋₃₀ alkyl group, especially a C₈₋₁₂ alkyl group; an alkyl group may also be a methyl, ethyl, propyl, isopropyl or butyl group. The expression “aryl” is defined as an aromatic residue, preferably substituted or optionally unsubstituted phenyl, benzyl, naphthyl, biphenyl or anthracene groups, which preferably have 6-24, more preferred 8-14 C ring atoms; the expression “alkenyl” can denote a C₂₋₁₀ alkenyl group, preferably a C₂₋₆ alkenyl group, which has the double bond or the double bonds at any desired location and may be substituted or unsubstituted; the expression “alkynyl” can denote a C₂₋₁₀ alkynyl group, preferably a C₂₋₆ alkynyl group, which has the triple bond or the triple bonds at any desired location and may be substituted or unsubstituted; the expression “alkoxy” can denote a C₁₋₅₀ alkyl-oxygen group, preferably a C₁₋₆ alkyl-oxygen group; the expression “alkenyloxy” can denote a C₂₋₁₀ alkenyl-oxygen group, preferably a C₂₋₆ alkenyl-oxygen group; the expression “alkynyloxy” can denote a C₂₋₁₀ alkynyl-oxygen group, preferably a C₂₋₆ alkynyl-oxygen group; the expression “carbocyclicoxy” can denote a C₃₋₁₂ carbocyclic-oxygen group; the expression “heteroaryloxy” can denote a heteroaryl-oxygen group, the expression “heterocyclicoxy” can denote a heterocyclic-oxygen group; the expression “substituted” can denote any desired substitution by one or more, preferably one or two, alkyl, alkenyl, alkynyl, mono- or multi-valent acyl, alkoxy, alkoxyacyl, alkenyloxy, alkynyloxy, carbocyclicoxy, heteroaryloxy, heterocyclicoxy, alkoxyalkyl groups, any monoether or polyether containing identical or different alkyl, aryl, alkenyl, alkynyl, carbocyclic, heteroaryl, heterocyclic residues, or any monothioether or polythioether containing identical or different alkyl, aryl, alkenyl, alkynyl, carbocyclic, heteroaryl, heterocyclic residues; the afore-mentioned substituents may in turn have one or more (but preferably zero) alkyl, alkenyl, alkynyl, mono- or multi-valent acyl, alkoxyacyl or alkoxyalkyl groups as side groups which are preferably not substituted themselves. Organic amines, amides, alcohols or acids, each having from 8 to 50 C atoms, preferably from 10 to 20 C atoms, can have the formulae (alkyl)₂N— or alkyl-NH—, —CO—N(alkyl)₂ or —CO—NH(alkyl), -alkyl-OH or -alkyl-COOH.

The expression urethanes can denote a compound of the formula R—NH—CO—OR″″, wherein R″″ is a substituted alkyl, acyl, alkenyl, alkynyl, carbocyclic, heteroaryl, heterocyclic or aryl residues. R is identical to the residue R of formula VII and is as defined for formula VII. Preferred for R″″ are unsubstituted or substituted alkyl residues, e.g. methyl, ethyl, tert-butyl, 2,2,2-trichloroethyl, 2-(trimethylsilyl)ethyl; unsubstituted or substituted alkenyl residues, e.g. allyl; unsubstituted or substituted aryl residues, e.g. phenyl, benzyl, 9-fluorenylmethyl.

All of the above residues or groups can—if possible—be branched or unbranched, unsubstituted or substituted with, e.g., 1, 2, 3, 4 or 5 substitutents, whereof 1 or 2 substituents are preferred.

The expression “peptide” for the definition of the residue R can denote any di-, tri-, tetra-, penta-, hexa-, or polypeptide. The peptide can be constituted of any amino acids or mimetics of amino acids or peptides.

The group AS-AS can be constituted of any two amino acids or mimetics thereof.

Examples of amino acids include: aspartic acid (Asp), glutamic acid (Glu), arginine (Arg), lysine (Lys), histidine (His), glycine (Gly), serine (Ser) and cysteine (Cys), threonine (Thr), asparagine (Asn), glutamine (Gln), tyrosine (Tyr), alanine (Ala), proline (Pro), valine (Val), isoleucine (Ile), leucine (Leu), methionine (Met), phenylalanine (Phe), tryptophan (Trp), hydroxyproline (Hyp), beta-alanine (beta-Ala), 2-amino octanoic acid (Aoa), azetidine-(2)-carboxylic acid (Ace), pipecolic acid (Pip), 3-amino propionic, 4-amino butyric and so forth, alpha-aminoisobutyric acid (Aib), sarcosine (Sar), ornithine (Orn), citrulline (Cit), homoarginine (Har), t-butylalanine (t-butyl-Ala), t-butylglycine (t-butyl-Gly), N-methylisoleucine (N-Melle), phenylglycine (Phg), cyclohexylalanine (Cha), norleucine (Nle), cysteic acid (Cya) and methionine sulfoxide (MSO), Acetyl-Lys, modified amino acids such as phosphoryl-serine (Ser(P)), benzyl-serine (Ser(Bzl)) and phosphoryl-tyrosine (Tyr(P)), 2-aminobutyric acid (Abu), aminoethylcysteine (AECys), carboxymethylcysteine (Cmc), dehydroalanine (Dha), dehydroamino-2-butyric acid (Dhb), carboxyglutaminic acid (Gla), homoserine (Hse), hydroxylysine (Hyl), cis-hydroxyproline (cis Hyp), trans-hydroxyproline (transHyp), isovaline (Iva), pyroglutamic acid (Pyr), norvaline (Nva), 2-aminobenzoic acid (2-Abz), 3-aminobenzoic acid (3-Abz), 4-aminobenzoic acid (4-Abz), 4-(aminomethyl)benzoic acid (Amb), 4-(aminomethyl)cyclohexanecarboxylic acid (4-Amc), Penicillamine (Pen), 2-Amino-4-cyanobutyric acid (Cba), cycloalkane-carboxylic acids.

Examples of ω̅-amino acids are e.g.: 5-Ara (aminoraleric acid), 6-Ahx (aminohexanoic acid), 8-Aoc (aminooctanoic acid), 9-Anc (aminovanoic acid), 10-Adc (aminodecanoic acid), 11-Aun (aminoundecanoic acid), 12-Ado (aminododecanoic acid).

Further amino acids may be: indanylglycine (Igl), indoline-2-carboxylic acid (Idc), octahydroindole-2-carboxylic acid (Oic), diaminopropionic acid (Dpr), diaminobutyric acid (Dbu), naphtylalanine (1-Nal), (2-Nal), 4-aminophenylalanin (Phe(4-NH₂)), 4-benzoylphenylalanine (Bpa), diphenylalanine (Dip), 4-bromophenylalanine (Phe(4-Br)), 2-chlorophenylalanine (Phe(2-Cl)), 3-chlorophenylalanine (Phe(3-Cl)), 4-chlorophenylalanine (Phe(4-Cl)), 3,4-chlorophenylalanine (Phe(3,4-C₁₂)), 3-fluorophenylalanine (Phe(3-F)), 4-fluorophenylalanine (Phe(4-F)), 3,4-fluorophenyl (Phe(3,4-F₂)), pentafluorophenylalanine (Phe(F₅)), 4-guanidinophenylalanine (Phe(4-guanidino)), homophenylalanine (hPhe), 3-jodophenylalanine (Phe(3-J)), 4-jodophenylalanine (Phe(4-J)), 4-methylphenylalanine (Phe(4-Me)), 4-nitrophenylalanine (Phe-4-NO₂)), biphenylalanine (Bip), 4-phosphonomethylphenylalanine (Pmp), cyclohexyglycine (Ghg), 3-pyridinylalanine (3-Pal), 4-pyridinylalanine (4-Pal), 3,4-dehydroproline (A-Pro), 4-ketoproline (Pro(4-keto)), thioproline (Thz), isonipecotic acid (Inp), 1,2,3,4,-tetrahydroisoquinolin-3-carboxylic acid (Tic), propargylglycine (Pra), 6-hydroxynorleucine (NU(6-OH)), homotyrosine (hTyr), 3-jodotyrosine (Tyr(3-J)), 3,5-dijodotyrosine (Tyr(3,5-J₂)), d-methyl-tyrosine (Tyr(Me)), 3-NO₂-tyrosine (Tyr(3-NO₂)), phosphotyrosine (Tyr(PO₃H₂)), alkylglycine, 1-aminoindane-1-carboxy acid, 2-aminoindane-2-carboxy acid (Aic), 4-amino-methylpyrrol-2-carboxylic acid (Py), 4-amino-pyrrolidine-2-carboxylic acid (Abpc), 2-aminotetraline-2-carboxylic acid (Atc), diaminoacetic acid (Gly(NH₂)), diaminobutyric acid (Dab), 1,3-dihydro-2H-isoinole-carboxylic acid (Disc), homocylcohexylalanin (hCha), homophenylalanin (hPhe oder Hof), trans-3-phenyl-azetidine-2-carboxylic acid, 4-phenyl-pyrrolidine-2-carboxylic acid, 5-phenyl-pyrrolidine-2-carboxylic acid, 3-pyridylalanine (3-Pya), 4-pyridylalanine (4-Pya), styrylalanine, tetrahydroisoquinoline-1-carboxylic acid (Tiq), 1,2,3,4-tetrahydronorharmane-3-carboxylic acid (Tpi), β-(2-thienyl)-alanine (Tha).

Proteinogenic amino acids are defined as natural protein-derived (α-amino acids. Non-proteinogenic amino acids are defined as all other amino acids, which are not building blocks of common natural proteins.

Peptide mimetics per se are known to a person skilled in the art. They are preferably defined as compounds which have a secondary structure like a peptide and optionally further structural characteristics; their mode of action is largely similar or identical to the mode of action of the native peptide; however, their activity (e.g. as an antagonist or inhibitor) can be modified as compared with the native peptide, especially vis à vis receptors or enzymes. Moreover, they can imitate the effect of the native peptide (agonist). Examples of peptide mimetics are scaffold mimetics, non-peptidic mimetics, peptoides, peptide nucleic acids, oligopyrrolinones, vinylogpeptides and oligocarbamates. For the definitions of these peptide mimetics see Lexikon der Chemie, Spektrum Akademischer Verlag Heidelberg, Berlin, 1999.

Further peptide mimetics are defined in J. Gante, Angew. Chemie, 1994, 106, 1780-1802; V. J. Hruby et al., Biopolymers, 1997, 219-266; D. Nöteberg et al., 2000, 43, 1705-1713.

Prodrugs of the compounds provided herein may also be included. In general, such prodrugs will be functional derivatives of the compounds which are readily convertible in vivo into the desired therapeutically active compound. Conventional procedures for the selection and preparation of suitable prodrug derivatives are described, for example, in “Design of Prodrugs”, ed. H. Bundgaard, Elsevier, 1985.

Such prodrugs can be cleaved and the active inhibitors can be released. This activation of the active inhibitors can be achieved both by chemical and enzymatic reactions. Esterases, proteases and peptidases serve to release the active inhibitors from the compounds according to the invention. Esterases, proteases and peptidases, which are suitable in such manner, are disclosed in WO 97/45117, U.S. Pat. Nos. 5,433,955, 5,614,379 and 5,624,894. Preferred proteases are aminopeptidases, dipeptidyl aminopeptidases, endoproteases, and endopeptidases. Especially preferred proteases for the release of the active inhibitors from the precursor of the present invention are aminopeptidase N, aminopeptidase P, pyroglutaminyl aminopeptidase, dipeptidyl peptidase IV and dipeptidyl peptidase IV-like enzymes. Such proteases and their specificity are described in Handbook of Proteolytic Enzymes, Eds. Barrett, A. J., Rawlings, N. D. and Woessner, J. F. Academic Press, New York 1998.

Also, inhibitors of cathepsin C may be, for example, cysteine protease inhibitors, such as peptidyl aldehydes (Woo J-T, et al., Bioorg. Med., Chem. Lett., 5:1501-1504 (1995), and Yasuma T, et al., J. Med. Chem., 41:4301-4308 (1998)); α-ketoesters, α-ketoamides (Hu R J, Bennett V, J. Biol. Chem., 266:18200-18205 (1991), and Li Z, et al., J. Med. Chem., 36:3472-3480 (1993)); acyloxymethyl ketones (Piura D H, et al., Biochem. J., 288:759-762 (1992), and Bromme D, et al., Biol. Chem., Hoppe-Seyler 375 (1994)); vinyl sulfones (Palmer J T, et al., J. Med. Chem., 38:3193-3196, and Bromme D, et al., Biochem. J., 315(Pt. 1) 85-89 (1996)); and epoxysuccinyl derivatives (Barrett A J, et al., Biochem. J., 201:189-198 (1982), Schaschke N, et al., Bioorg. Med. Chem. Lett., 5:1789-1797 (1997), and Gour-Salin B J, et al., J. Med. Chem., 36:720-725 (1993)).

The natural inhibitors of cathepsin C, as well as of other cysteine protease cathepsins include for example, cystatin (Nicklin M J H, et al., Biochem J., 223:245-253 (1984)); leupeptin (McGuire M J, et al., Arch. Biochem. Biophys., 295:280-288 (1992)); and E-64 (Barrett A J, et al., Biochem. J., 201:189-198 (1982)). These inhibitors inhibit cathepsin C at high inhibitor concentrations.

Additional inhibitors of cathepsin C include dipeptide acyloxymethyl ketones, dipeptide fluoromethyl ketones, and dipeptide vinyl sulfones (VS), such as Ala-Hph-VS-Ph (Kam C M, et al., Arch. Biochem. Biophys., 427(2):123-134 (July 2004), which is incorporated by reference herein in its entirety), examples of which are shown in Table C below:

TABLE C

| | |
|---|---|
| Gly-Phe CH₂OCOAr (An Acyloxymethyl Ketone) | 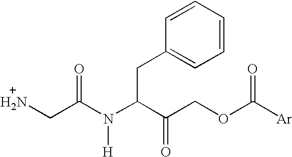 |
| Gly-Phe-VS-R, where R = CH₃, Ph, or 4-Cl—Ph (A Vinyl Sulfone) | 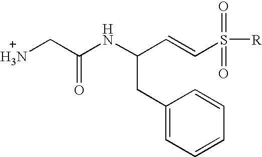 |
| Gly-Phe-CH₂F (A Fluoromethyl Ketone) | 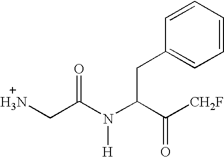 |

Specific examples of dipeptide acyloxymethyl ketones include:

N-Glycyl-L-phenylalanyl(benzoyloxy)methane hydrochloride (Gly-Phe-CH₂OCOPh.HCl);

Gly-Phe-CH₂OCO-(2,4,6-trimethyl)C₆H₂;

Gly-Phe-CH₂OCO-(2,6-dichloro)C₆H₉; and

N-Glycyl-L-glutamyl(benzoyloxy)methane hydrochloride (Gly-Glu-CH₂OCOPh.HCl).

Specific examples of dipeptide vinyl sulfones include:

Diethyl phenylsulfonylmethane phosphonate (Ph-SO₂—CH₂—PO(OEt) 2);

3-Amino-5-carboxyl-1-(phenylsulfonyl)-1-pentene hydrochloride (Glu-VS-Ph.HCl);

Glu-Phe-VS-Ph;

Gly-Phe-VS-Ph;

Ala-Phe-VS-Ph;

Val-Phe-VS-Ph;

Leu-Phe-VS-Ph;

Lys(Z)-Phe-VS-Ph;

Lys(Fla-Adp)-Phe-VS-Ph;

Ala-Hph-VS-Ph;

Nva-Hph-VS-Ph;

Glu-Hph-VS-Ph;

Ala-Lys(Fla-Adp)-VS-Ph;

Glu-Glu-VS-Ph;

Gly-Glu-VS-Ph;

Thr-Glu-VS-Ph;

Gly-Phe-VS—CH₃;

Ala-Phe-VS—CH₃;

Val-Phe-VS—CH₃

Leu-Phe-VS—CH₃;

Val-Phe-VS-Ph-Cl;

Leu-Phe-VS-Ph-Cl;

Arg-Phe-VS-Ph;

Arg-Hph-VS-Ph;

Boc-Gly-Phe-VS-Ph;

Boc-Thr(OtBu)-Glu-VS-Ph;

Phe-VS-Ph; and

3-(N-L-Glutamyl)amino-5-carboxyl-1-(phenylsulfonyl)-1-pentene hydrochloride (Glu-Glu-VS-Ph.HCl). As found by Kam et al. supra, the dipeptide vinyl sulfones with the Ala-Hph or Nva-Hph sequence were the best inhibitors of purified cathepsin C. Gly-Phe-CHN2 was also found to be a potent inhibitor of cathepsin C.

Specific example of dipeptide fluoromethyl ketones include N-Glycyl-L-phenylalanyl fluoromethane hydrochloride (Gly-Phe-CH₂F.HCl).

Cathepsin C inhibitors may also include phosphonic tripeptides and analogues.

Cathepsin C inhibitors also include dipeptide nitriles (Bondebjerg J, et al., Bioorg Med Chem Lett., 16(13):3614-3617 (July 2006).

Dipeptide nitriles are also considered to be inhibitors of other cysteine cathepsins. Dipeptide nitriles were described in great detail in U.S. Pat. No. 6,353,071.

The general definitions used to describe the dipeptide nitrile compounds of Formula I, II, II′, III, III′, III″, IV, V, V′ and V″, as defined below, have the following meaning in this disclosure, unless otherwise specified.

The term “lower” in connection with organic radicals or compounds respectively defines such as branched or unbranched with up to and including 7, preferably up to and including 4 and advantageously one or two carbon atoms.

A lower alkyl group is branched or unbranched and contains 1 to 7 carbon atoms, preferably 1-4 carbon atoms. Lower alkyl represents, for example, methyl, ethyl, propyl, butyl, isopropyl, isobutyl, or tert-butyl.

Lower alkenyl represents either straight chain or branched alkenyl of 2 to 7 carbon atoms, preferably 24 carbon atoms, e.g., as vinyl, propenyl, isopropenyl, butenyl, isobutenyl, or butadienyl.

Lower alkynyl represents either straight chain or branched alkynyl of 2 to 7 carbon atoms, preferably 2-4 carbon atoms, e.g., as acetylenyl, propynyl, isopropynyl, butynyl, or isobutynyl.

Lower alkyl, lower alkenyl and lower alkynyl may be substituted by up to 3 substituents selected from lower alkoxy, aryl, hydroxy, halogen, cyano, or trifluoromethyl.

Lower alkylene represents either straight chain or branched alkylene of 1 to 7 carbon atoms and represents preferably straight chain alkylene of 1 to 4 carbon atoms, e.g., a methylene, ethylene, propylene or butylene chain, or said methylene, ethylene, propylene or butylene chain mono-substituted by C₁-C₃-alkyl (advantageously methyl) or disubstituted on the same or different carbon atoms by C₁-C₃-alkyl (advantageously methyl), the total number of carbon atoms being up to and including 7.

A lower alkoxy (or alkyloxy) group preferably contains 14 carbon atoms, advantageously 1-3 carbon atoms, and represents, for example, ethoxy, propoxy, isopropoxy, or most advantageously methoxy.

Halogen (halo) preferably represents chloro or fluoro but may also be bromo or iodo.

An acyl group as represented by R₃₀ is preferably derived from an organic carbonic acid, an organic carboxylic acid, a carbamic acid or an organic sulfonic acid.

Acyl which is derived from a carboxylic acid represents, for example, carbocyclic or heterocyclic aroyl, cycloalkylcarbonyl, (oxa or thia)-cycloalkylcarbonyl, lower alkanoyl, (lower alkoxy, hydroxy or acyloxy)-lower alkanoyl, (mono- or di-carbocyclic or heterocyclic)-(lower alkanoyl or lower alkoxy-, hydroxy- or acyloxy-substituted lower alkanoyl), or biaroyl.

Carbocyclic aroyl represents, for instance, benzoyl, benzoyl substituted, by one to three substituents selected independently from e.g., halo, trifluoromethyl, lower alkyl, lower alkoxy, hydroxy, methylenedioxy, nitro, di-lower alkylamino, cyano, or carbocyclic aroyl represents, e.g., 1- or 2-naphthoyl.

Heterocyclic aroyl represents, for instance, 2-, 3- or 4-pyridylcarbonyl (such as nicotinoyl), furoyl, thienoyl, oxazoloyl, isoxazoloyl, quinoxaloyl, each optionally substituted by, e.g., halo, lower alkyl, lower alkoxy or nitro.

(Oxa- or thia)-cyclolalkylcarbonyl is, for example, tetrahydrofuranoyl or tetrahydrothienoyl. Dicarbocyclic or heterocyclic)aryl-lower alkanoyl is, for example, diphenylacetyl or dipyridylacetyl.

Aryl-(lower alkoxy, hydroxy or acyloxy substituted) lower alkanoyl is, for example, phenyl-(2-alkoxy, hydroxy or acyloxy)-acetyl.

Biaroyl is, for example, 2, 3 or 4-biphenylcarbonyl.

Acyl which is derived from an organic carbonic acid is, for example, alkoxycarbonyl, especially lower alkoxycarbonyl, which is unsubstituted or substituted by carbocyclic or heterocyclic aryl or is cycloalkoxycarbonyl, especially C₃-C₇-cycloalkyloxycarbonyl, which is unsubstituted or substituted by lower alkyl.

Acyl which is derived from a carbamic acid is, for example, aminocarbonyl which is optionally substituted on nitrogen by one or two of lower alkyl, carbocyclic or heterocyclic aryl-lower alkyl, carbocyclic or heterocyclic aryl, or by lower alkylene or lower alkylene interrupted by O or S.

Acyl which is derived from an organic sulfonic acid represents, for example, lower alkylsulfonyl, carbocyclic or heterocyclic arylsulfonyl, carbocyclic or heterocyclic aryl-lower alkysulfonyl, in which aryl is, e.g., phenyl, naphthyl or thienyl, such being optionally substituted by, for example, lower alkyl, lower alkoxy, halo, nitro, trifluoromethyl, carboxyl or lower alkoxycarbonyl.

Aryl represents carbocyclic or heterocyclic aryl.

Carbocyclic aryl represents monocyclic, bicyclic or tricyclic aryl, for example phenyl or phenyl mono-, di- or tri-substituted by one, two or three radicals selected from lower alkyl, lower alkoxy, aryl, hydroxy, halogen, cyano, trifluoromethyl, lower alkylenedioxy and oxy-C₂-C₃-alkylene; or 1- or 2-naphthyl; or 1- or 2-phenanthrenyl. Lower alkylenedioxy is a divalent substituent attached to two adjacent carbon atoms of phenyl, e.g., methylenedioxy or ethylenedioxy. Oxy-C₂-C₃-alkylene is also a divalent substituent attached to two adjacent carbon atoms of phenyl, e.g., oxyethylene or oxypropylene. An example for oxy-C₂-C₃-alkylene-phenyl is 2,3-dihydrobenzofuran-5-yl.

Preferred as carbocyclic aryl is naphthyl, phenyl or phenyl mono- or disubstituted by lower alkoxy, phenyl, halogen, lower alkyl or trifluoromethyl, especially phenyl or phenyl mono- or disubstituted by lower alkoxy, halogen or trifluoromethyl, and in particular phenyl.

Examples of substituted phenyl groups as R are, e.g., 4-chlorophen-1-yl, 3,4-dichlorophen-1-yl, 4-methoxyphen-1-yl, 4-methylphen-1-yl, 4-aminomethylphen-1-yl, 4-methoxyethylaminomethylphen-1-yl, 4-hydroxyethylaminomethylphen-1-yl, 4-hydroxyethyl-(methyl)-aminomethylphen-1-yl, 3-aminomethylphen-1-yl, 4-N-acetylaminomethylphen-1-yl, 4-aminophen-1-yl, 3-aminophen-1-yl, 2-aminophen-1-yl, 4-phenyl-phen-1-yl, 4-(imidazol-1-yl)-1-yl, 4-(imidazol-1-ylmethyl)-phen-1-yl, 4-(morpholin-1-yl)-phen-1-yl, 4-(morpholin-1-ylmethyl)-phen-1-yl, 4-(2-methoxyethylaminomethyl)-phen-1-yl and 4-(pyrrolidin-1-ylmethyl)-phen-1-yl, 4-(2-thiophenyl)-phen-1-yl, 4-(3-thiophenyl)-phen-1-yl, 4-(4-methylpiperazin-1-yl)-phen-1-yl, and 4-(piperidinyl)-phenyl and 4-(pyridinyl)-phenyl optionally substituted in the heterocyclic ring.

Heterocyclic aryl represents monocyclic or bicyclic heteroaryl, such as, pyridyl, indolyl, quinoxalinyl, quinolinyl, isoquinolinyl, benzothienyl, benzofuranyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, or any said radical substituted, especially mono- or di-substituted, by, e.g., lower alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represent preferably 3-benzopyranyl or 3-benzothiopyranyl, respectively. Thiazolyl represents preferably 2- or 4-thiazolyl, advantageously 4-thiazolyl. Triazolyl is preferably 1-, 2- or 5-(1,2,4-triazolyl). Tetrazolyl is preferably 5-tetrazolyl.

Preferably, heterocyclic aryl is pyridyl, indolyl, quinolinyl, pyrrolyl, thiazolyl, isoxazolyl, triazolyl, tetrazolyl, pyrazolyl, imidazolyl, thienyl, or any said radical substituted, especially mono- or di-substituted, by lower alkyl or halogen; and in particular pyridyl.

Arylene (Ar in Formula III) is an aryl linking group in which aryl is heterocyclic or carbocyclic aryl, preferably monocyclic as defined above.

A heterocyclic aryl linking group is for instance (but not limited thereto) 1,3-pyrazolyl, 2,4- or 2,5-pyridyl or 1,4-imidazolyl in which the groups as depicted in Formula III are attached to the ring at the indicated positions.

A carbocyclic aryl linking group is for instance (but not limited thereto) optionally substituted phenyl in which the two groups as depicted in Formula I are attached ortho, meta or para to each other.

Biaryl may be carbocyclic biaryl, preferably biphenyl, namely 2, 3 or 4-biphenyl, advantageously 4-biphenyl, each optionally substituted by, e.g., lower alkyl, lower alkoxy, halogen, trifluoromethyl or cyano, or heterocyclic-carbocyclic biaryl, preferably thienylphenyl, pyrrolylphenyl and pyrazolylphenyl.

Cycloalkyl represents a saturated cyclic hydrocarbon optionally substituted by lower alkyl which contains 3 to 10 ring carbons and is advantageously cyclopentyl, cyclohexyl, cycloheptyl or cyclooctyl optionally substituted by lower alkyl.

Bicycloalkyl may be, for example, norbornyl.

Heterocyclic represents a saturated cyclic hydrocarbon containing one or more, preferably 1 or 2, hetero atoms selected from O, N or S, and from 3 to 10, preferably 5 to 8, ring atoms; for example, tetrahydrofuranyl, tetrahydrothienyl, tetrahydropyrrolyl, piperidinyl, piperazinyl, or morpholino.

Aryl-lower alkyl represents preferably (carbocyclic aryl or heterocylic aryl)-lower alkyl.

Carbocyclic aryl-lower alkyl represents preferably straight chain or branched aryl-C₁₋₄-alkyl in which carbocyclic aryl has meaning as defined above, e.g., benzyl or phenyl-(ethyl, propyl or butyl), each unsubstituted or substituted on phenyl ring as defined under carbocyclic aryl above, advantageously optionally substituted benzyl, e.g., benzyl substituted or phenyl lay lower alkyl.

Heterocyclic aryl-lower alkyl represents preferably straight chain or branched heterocyclic aryl-C₁₋₄-alkyl in which heterocyclic aryl has meaning as defined above, e.g., 2-, 3- or 4-pyridylmethyl or (2,3- or 4-pyridyl)-(ethyl, propyl or butyl); or 2- or 3-thienylmethyl or (2- or 3-thienyl)-(ethyl, propyl or butyl); 2-, 3- or 4-quinolinylmethyl or (2-, 3- or 4-quinolinyl)-(ethyl, propyl or butyl); or 2- or 4-thiazolylmethyl or (2- or 4-thiazolyl)-(ethyl, propyl or butyl).

Cycloalkyl-lower alkyl represents, e.g., (cyclopentyl- or cyclohexyl)-(methyl or ethyl).

Biaryl-lower alkyl represents, e.g., 4-biphenylyl-(methyl or ethyl).

Acyl as in acyloxy is derived from an organic carboxylic acid, carbonic acid or carbarnic acid. Acyl represents, e.g., lower alkanoyl, carbocyclic aryl-lower alkanoyl, lower alkoxycarbonyl, aroyl, di-lower alkylaminocarbonyl or di-lower alkylamino-lower alkanoyl. Preferably, acyl is lower alkanoyl.

Lower alkanoyl represents, e.g., C₁₋₇-alkanoyl including formyl, and is preferably C₂₋₄-alkanoyl such as acetyl or propionoyl.

Aroyl represents, e.g., benzoyl or benzoyl mono- or di-substituted by one or two substituents selected from lower alkyl, lower alkoxy, halogen, cyano and trifluoromethyl; or 1- or 2-naphthoyl; and also, e.g., pyridylcarbonyl.

Lower alkoxycarbonyl represents preferably C₁₋₄-alkoxycarbonyl, e.g., ethoxycarbonyl.

Esterified carboxyl is carboxyl derivatized as a pharmaceutically acceptable ester, such as, lower alkoxycarbonyl, benzyloxycarbonyl, or allyloxycarbonyl.

Amidated carboxyl is carboxyl derivatized as a pharmaceutically acceptable amide, such as aminocarbonyl, mono- or di-lower alkylaminocarbonyl.

Pharmaceutically acceptable salts of the acidic dipeptide nitriles are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

In one embodiment, specifically, an N-terminal-substituted dipeptide nitrile, i.e. a dipeptide in which C-terminal carboxy group of the dipeptide is replaced by a nitrile group (—C≡N) and in which the N-terminal nitrogen atom is substituted via a peptide or pseudopeptide linkage which optionally additionally comprises a -methylene-hetero atom-linker or an additional hetero atom, directly by aryl, lower alkyl, lower alkenyl, lower alkynyl or heterocyclyl, or a physiologically-acceptable and -cleavable ester or salts thereof, may be used to inhibit cathepsins.

The dipeptide nitrite may conveniently comprise α-amino acid residues, including both natural and unnatural α-amino acid residues. Herein the “natural α-amino acid residues” denote the 20 amino acids obtainable by translation of RNA according to the genetic code or the corresponding nitrites thereof, as appropriate. “Unnatural α-amino acid residues” are α-amino acids which have α-substituents other than those found in “natural α-amino acid residues”. Preferred α-amino acid residues, as the C-terminal amino acid residue of the dipeptide nitrite, are the nitrites of tryptophan, 2-benzyloxymethyl-2-amino-acetic acid, 2,2-dimethyl-2-amino-acetic acid, 2-butyl-2-amino-acetic acid, methionine, leucine, lysine, alanine, phenylalanine, and glycine and derivatives thereof, e.g. as hereinafter described. Preferred amino acid residues as the N-terminal amino acid residue of the dipeptide nitrite are 1-amino-cyclohexanecarboxylic acid, 1-amino-cycloheptanecarboxylic acid, phenylalanine, histidine, tryptophan and leucine and derivatives thereof, e.g., as hereinafter described.

The aryl, lower alkyl, lower alkenyl, lower alkynyl or heterocyclyl substituent (hereinafter referred to as R) is attached to the N-terminal nitrogen atom of the dipeptide via a peptide linkage, i.e. as R—C(O)—NH—, or via a pseudopeptide linkage. Suitable pseudopeptide linkages include sulphur in place of oxygen and sulphur and phosphorous in place of carbon, e.g., as R—C(S)—NH—, R—S(O)—NH—, R—S(O)₂—NH— or R—P(O)₂—NH and analogues thereof. Additionally the peptide or pseudopeptide linkage between the R substituent and the N-terminal nitrogen atom may comprise an additional hetero atom, e.g., as R—Het-C(O)—NH—, or a -methylene-hetero atom-linker, e.g., as R-Het-CH₂—C(O)—NH— or R—CH₂-Het-C(O)—NH—, wherein Het is a hetero atom selected from O, N or S, and pseudopeptide containing alternatives thereof, e.g., as defined above. When the linkage between the aryl substituent and the N-terminal nitrogen atom comprises a -methylene-hetero atom-linker, the methylene group and the hetero atom may be optionally further substituted, e.g., as hereinafter described.

The R substituent may be further substituted, e.g., by up to 3 substituents selected from halogen, hydroxy, amino, nitro, optionally substituted C₁₋₄ alkyl (e.g., alkyl substituted by hydroxy, alkyloxy, amino, optionally substituted alkylamino, optionally substituted dialkylamino, aryl or heterocyclyl), C₁₋₄ alkoxy, C₂₋₆ alkenyl, CN, trifluoromethyl, trifluoromethoxy, aryl, (e.g., phenyl or phenyl substituted by CN, CF₃, halogen, OCH₃), aryloxy, (e.g., phenoxy or phenoxy substituted by CN, CF₃, halogen, OCH₃), benzyloxy or a heterocyclic residue.

A cathepsin inhibitor may be a dipeptide nitrile of Formula I, or a physiologically-acceptable and -cleavable ester or a salt thereof.

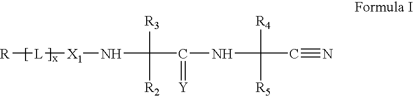

wherein:

R is optionally substituted (aryl, lower alkyl, lower alkenyl, lower alkynyl, or heterocyclyl);

R₂ and R₃ are independently hydrogen, or optionally substituted [lower alkyl, cycloalkyl, bicycloalkyl, or (aryl, biaryl, cycloalkyl or bicycloalkyl)-lower alkyl]; or

R₂ and R₃ together represent lower alkylene, optionally interrupted by O, S or NR₆, so as to form a ring with the carbon atom to which they are attached wherein R₆ is hydrogen, lower alkyl or aryl-lower alkyl; or

either R₂ or R₃ are linked by lower alkylene to the adjacent nitrogen to form a ring;

R₄ and R₅ are independently H, or optionally substituted (lower alkyl, aryl-lower alkyl), —C(O)OR₇, or —C(O)NR₇R₈,

wherein

R₇ is optionally substituted (lower alkyl, aryl, aryl-lower alkyl, cycloalkyl, bicycloalkyl or heterocyclyl), and

R₈ is H, or optionally substituted (lower alkyl, aryl, aryl-lower alkyl, cycloalkyl, bicycloalkyl or heterocyclyl), or

R₄ and R₅ together represent lower alkylene, optionally interrupted by O, S or NR₆, so as to form a ring with the carbon atom to which they are attached wherein R₆ is hydrogen, lower alkyl or aryl-lower alkyl, or

R₄ is H or optionally substituted lower alkyl and R₅ is a substituent of formula —X₂—(Y₁)ₙ—(Ar)ₚ-Q-Z

Wherein

Y₁ is O, S, SO, SO₂, N(R₆)SO₂, N—R₆, SO₂ NR₆, CONR₆ or NR₆ CO;

n is zero or one;

p is zero or one;

X₂ is lower alkylene; or when n is zero, X₂ is also C₂-C₇-alkylene interrupted by O, S, SO, SO₂, NR₆, SO₂ NR₆, CONR₆ or NR₆ CO;

wherein R₆ is hydrogen, lower alkyl or aryl-lower alkyl;

Ar is arylene;

Z is hydroxy, acyloxy, carboxyl, esterified carboxyl, amidated carboxyl, aminosulfonyl, (lower alkyl or aryl-lower alkyl)aminosulfonyl, or (lower alkyl or aryl-lower alkyl)sulfonylaminocarbonyl; or Z is tetrazolyl, triazolyl or imidazolyl;

Q is a direct bond, lower alkylene, Y₁-lower alkylene or C₂-C₇-alkylene interrupted by Y₁;

X₁ is —C(O)—, —C(S)—, —S(O)—, —S(O)₂—, —P(O)(OR₆)—

wherein R₆ is as defined above;

Y is oxygen or sulphur;

L is optionally substituted -Het-, -Het-CH₂— or —CH₂-Het-,

wherein Het is a hetero atom selected from O, N or S, and

x is zero or one;

and aryl in the above definitions represents carbocyclic or heterocyclic aryl, for use as a pharmaceutical;

a pharmaceutical composition comprising a compound of Formula I as defined above as an active ingredient;

a method of treating a patient suffering from or susceptible to a disease or medical condition in which a cathepsin is implicated, comprising administering an effective amount of a compound of Formula I as defined above to the patient; and

use of a compound of Formula I as defined above for the preparation of a medicament for therapeutic or prophylactic treatment of a disease or medical condition in which a cathepsin is implicated.

A cathepsin inhibitor may be, for example, a dipeptide nitrile of Formula I as defined above

provided that when R is lower alkyl not substituted by aryl,

one of R₄ or R₅ is a substituent of formula —X₂—(Y₁)ₙ—(Ar)ₚ-Q-Z,

provided that when x is one, L is —O—, or —CH₂—O— and X₁ is —C(O)—,

either one of R₄ or R₅ is a substituent of formula —X₂—(Y₁)ₙ—(Ar)ₚ-Q-

Z, or R is not unsubstituted phenyl,

provided that when R₂═R₄═R₅═H, x is zero and X₁ is —C(O)—,

R₃ is not H, —CH₃, —CH(CH₃)₂, —CH₂—CH—(CH₃)₂, —CH₂—COOH, or —CH₂—COO—CH₂—CH₃, when R is unsubstituted phenyl,

R₃ is not H, —CH(CH₃)₂, or —CH₂—CH—(CH₃)₂, when R is 4-aminophenyl or 4-nitrophenyl,

R₃ is not H when R is 3-aminophenyl, 3-nitrophenyl 2-chloropyridin-4-yl, or vinyl or

R₃ is not —CH₂—CH₂—S—CH₃ when R is pyridin-3-yl or 2-chloropyridin4-yl,

provided that when R₂═R₃═R₄═H, x is zero and X₁ is —C(O)— and R is phenyl,

R₅ is not —CH(CH₃)₂,

provided that when R₃═R₄═H, R₅ is —CH₂—CH₂—COOH, x is zero and X₁ is —C(O)—,

R₂ does not form a heterocyclic ring with the adjacent nitrogen atom, and

provided that when R₂═R₃═R₄═R₅═H, x is zero and X₁ is —SO₂

R is not 4-methylphenyl.

In Formula I, R, R₂, R₃, R₄, R₅ and L may be further substituted by one or more, e.g., up to 3, substituents independently selected from lower alkyl, aryl, aryl-lower alkyl, cycloalkyl, heterocyclyl, —CN, -halogen, —OH, —NO₂, —NR₉R₁₀, —X₃—R₇, lower alkyl-X₃—R₈, halo-substituted lower alkyl,

wherein R₇ and R₈ are as defined above,

X₃ is —O—, —S—, —NR₈—, —C(O)—, —C(S)—, —S(O)—, —S(O)₂—, —C(O)O—, —C(S)O—, —C(O)NR₈—,

wherein R₈ is as defined above,

R₉ and R₁₀ are independently as defined above for R₈, or —X₄—R₈,

wherein X₄ is —C(O)—, —C(S)—, —S(O)—, —S(O)₂—, —C(O)O—, —C(S)O—, —C(O)NR₆—

wherein R₆ and R₇ are as defined above, or

R₈ and R₁₀ together with N form a heteroaryl group or a saturated or unsaturated heterocycloalkyl group, optionally containing one or more additional heteroatoms selected from O, N or S.

Compounds of Formula I exhibit valuable pharmacological properties in mammals, in particular as cysteine cathepsin inhibitors. By appropriate choice of groups R, R₂, R₃, R₄, R₅, X₁, Y and L, the relative selectivity of the compounds as inhibitors of the various cysteine cathepsin types, e.g., cathepsins B, K, L and S may be altered, e.g., to obtain inhibitors which selectively inhibit a particular cathepsin type or combination of cathepsin types.

A cathepsin inhibitor may be, for example, a dipeptide nitrile of Formula II, or a physiologically-acceptable and -cleavable ester or a salt thereof

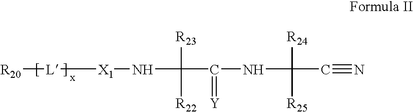

wherein:

R₂₀ is optionally substituted (aryl, aryl-lower alkyl, lower alkenyl, lower alkynyl, heterocyclyl, or heterocyclyl-lower alkyl);

R₂₂ is H, or optionally substituted lower alkyl, and

R₂₃ is optionally substituted (lower alkyl, aryl-lower alkyl, or cyloalkyl-lower alkyl) or

R₂₂ and R₂₃ together with the carbon atom to which they are attached form an optionally substituted (cycloalkyl group or heterocycloalkyl group);

R₂₄ and R₂₅ are independently H, or optionally substituted (lower alkyl, or aryl-lower alkyl), —C(O)OR₇, or —C(O)NR₇R₈

wherein R₇ and R₈ are as defined above, or

R₂₄ and R₂₅, together with the carbon atom to which they are attached form an optionally substituted (cycloalkyl group or heterocycloalkyl group);

X₁ is as defined above;

Y is oxygen or sulphur;

L′ is optionally substituted (-Het-CH₂— or —CH₂-Het-),

wherein Het is a hetero atom selected from O, N or S, and

x is 1 or 0,

provided that when x is one, L is —CH₂—O— and X₁ is —C(O)—,

R₂₀ is not unsubstituted phenyl,

provided that when R₂₂═R₂₄═R₂₅═H, x is zero and X, is —C(O)—,

R₂₃ is not H, —CH₃, —CH(CH₃)₂, —CH₂—CH—(CH₃)₂, —CH₂—COOH, or —CH₂—COO—CH₂—CH₃, when R₂₀ is unsubstituted phenyl,

R₂₃ is not H, —CH(CH₃)₂, or —CH₂—CH—(CH₃)₂, when R₂₀ is 4-aminophenyl or 4-nitrophenyl,

R₂₃ is not H when R₂₀ is 3-aminophenyl, 3-nitrophenyl 2-chloropyridin-4-yl, or vinyl, or

R₂₃ is not —CH₂—CH₂—S—CH₃ when R₂₀ is pyridin-3-yl or 2-chloropyridin-4-yl,

provided that when R₂₂═R₂₃═R₂₄═H, x is zero and X₁ is —C(O)— and

R₂₀ is phenyl,

R₂₅ is not —CH(CH₃)₂,

provided that when R₂₃═R₂₄═H, R₂₅ is —CH₂—CH₂—COOH, x is zero and X₁ is —C(O)—,

R₂₂ does not form a heterocyclic ring with the adjacent nitrogen atom, and

provided that when R₂₂═R₂₃═R₂₄═R₂₅═H, x is zero and X₁ is —SO₂—,

R₂₀ is not 4-methylphenyl.

Compounds of Formula II are typically inhibitors of cathepsins K, L or S, especially selective inhibitors of cathepsin K or cathepsin L or cathepsin S, or in some case inhibitors of, e.g., cathepsins L and S.

The substituents of the compounds of Formula II have the following preferred significances. Preferred compounds of Formula II comprise compounds having preferred substituents, singly or in any combination. Preferably when R₂₀ comprises aryl, the aryl is optionally substituted (phenyl, naphthylenyl, phenanthrenyl, thiophenyl, furanyl, pyrrolyl, pyrazolyl, thiazolyl, pyridinyl, indolyl, quinolinyl, isoquinolinyl, benzothienyl and benzofuranyl).

Preferably R₂₂ is hydrogen.

Preferably R₂₃ is optionally substituted (lower alkyl, aryl-lower alkyl or cycloalkyl-lower alkyl), or R₂₃ and R₂₂ together with the carbon atom to which they are attached form a C₅-C₈, especially a C₆ or C₇, cycloalkyl group. More preferably R₂₃ is —CH₂—CH(CH₃)₂, or optionally substituted benzyl, cyclohexylmethyl, naphthalenylmethyl, indolylmethyl, benzothienylmethyl or benzofuranylmethyl, or R₂₃ and R₂₂ together with the carbon atom to which they are attached form a cyclohexane ring.

Preferred significances for R₂₄ and R₂₅ are:

R₂₄ and R₂₅ are both H or —CH₃, or

R₂₄ is H and R₂₅ is aryl-lower alkyl, lower alkyl, both optionally substituted by up to 3 substituents selected from amino, halogen (e.g., fluorine or preferably chlorine) or S—CH₃, or

R₂₄ and R₂₅ together with the carbon atom to which they are attached form a C₃-C₇ cycloalkyl ring.

More preferably R₂₄ is H and R₂₅ is optionally substituted (—CH₂-phenyl, —CH₂-indolyl, —(CH₂)₂—S—CH₃, —CH₂—CH(CH₃)₂, —(CH₂)₄—NH₂ or —(CH₂)₃—CH₃), or yet more preferably R₄ and R₅ are both —CH₃, or especially R₄ and R₅ are both H.

Preferably —X₁— is —C(O)—.

Preferably Y is ═O.

Preferably either x is 0, or when x is 1 L′ is —CH₂—O—, —NH—CH₂—O—CH₂— or —S—CH₂.

A cathepsin inhibitor may be, for example, a dipeptide nitrile of Formula II′ or a physiologically-acceptable and -cleavable ester or a salt thereof.

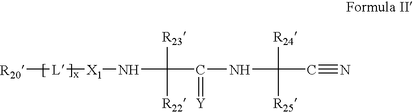

wherein:

R₂₀′ is optionally substituted (C₆-C₁₈ aryl or C₄-C₁₈ heteroaryl);

R₂₂′ is H, or optionally substituted C₁-C₈ alkyl, and

R₂₃′ is optionally substituted (C₂-C₈ alkyl, or C₇-C₁₄ aralkyl), or

R₂₂′ and R₂₃′ together with the carbon atom to which they are attached form an optionally substituted (C₃-C₈ cycloalkyl group or C₄-C₇ heterocycloalkyl group);

R₂₄′ and R₂₅′ are independently H, or optionally substituted (C₁-C₈ alkyl, C₇-C₁₄ aralkyl, or C₅-C₁₄ heteroaralkyl), —C(O)OR₆′, or —C(O)NR₆′R₇′

wherein

R₆′ is optionally substituted (C₁-C₈ alkyl, C₇-C₁₄ aralkyl, C₃-C₈ cycloalkyl, C₄-C₇ heterocycloalkyl, C₅-C₁₄ heteroaralkyl, C₆-C₁₄ aryl, or C₄-C₁₄ heteroaryl), and

R₇′ is H, or optionally substituted (C₁-C₈ alkyl, C₇-C₁₄ aralkyl, C₃-C₈ cycloalkyl, C₄-C₇ heterocycloalkyl, C₅-C₁₄ heteroaralkyl, C₆-C₁₄ aryl, or C₄-C₁₄ heteroaryl), or

R₂₄′ and R₂₅′ together with the carbon atom to which they are attached form an optionally substituted (C₃-C₈ cycloalkyl group or C₄-C₇ heterocycloalkyl group);

X₁ is —C(O)—, —C(S)—, —S(O)—, —S(O)₂—, —P(O)(OR₆′)—

wherein R′ is as defined above;

Y is oxygen or sulphur;

L′ is optionally substituted (-Het-CH₂— or —CH₂-Het-),

wherein Het is a hetero atom selected from O, N or S, and x is 1 or 0,

provided that when x is one, L′ is —CH₂—O— and X₁ is —C(O)—

R₂₀′ is not unsubstituted phenyl,

provided that when R₂₂′═R₂₄′═R₂₅′═H, x is zero and X₁ is —C(O)—,

R₂₃′ is not H, —CH₃, —CH(CH₃)₂, —CH₂—CH—(CH₃)₂, —CH₂—COOH, or —CH₂—COO—CH₂—CH₃, when R₂₀′ is unsubstituted phenyl,

R₂₃′ is not H, —CH(CH₃)₂, or —CH₂—CH—(CH₃)₂, when R₂₀′ is 4-aminophenyl or 4-nitrophenyl,

R₂₃′ is not H when R₂₀′ is 3-aminophenyl, 3-nitrophenyl, 2-chloropyridin-4-yl, or vinyl, or

R₂₃′ is not —CH₂—CH₂—S—CH₃ when R₂₀′ is pyridin-3-yl or 2-chloropyridin-4-yl,

provided that when R₂₂′═R₂₃′═R₂₄′═H, x is zero and X₁ is —C(O)— and

R₂₀′ is phenyl,

R₂₅′ is not —CH(CH₃)₂,

provided that when R₂₃′═R₂₄′═H, R₂₅′ is —CH₂—CH₂—COOH, x is zero and X₁ is —C(O)—,

R₂₀′ does not form a heterocyclic ring with the adjacent nitrogen atom, and

provided that when R₂₂′═R₂₃′═R₂₄′═R₂₅′ H, x is zero and X₁ is —SO₂—,

R₂₀′ is not 4-methylphenyl.

Compounds of Formula II′ are typically selective inhibitors of cathepsin K.

A cathepsin inhibitor may be, for example, a dipeptide nitrile of Formula III

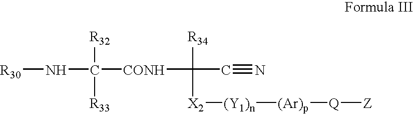

wherein:

R₃₀ is an acyl group derived from an organic carboxylic, carbonic, carbamic or sulfonic acid;

R₃₂ and R₃₃ are independently hydrogen, lower alkyl, cycloalkyl, bicycloalkyl, or (aryl, biaryl, cycloalkyl or bicycloalkyl)-lower alkyl; or R₃₂ and R₃₃ together represent lower alkylene so as to form a ring together with the carbon to which they are attached;

R₃₄ is hydrogen or lower alkyl; X₂, Y₁, Ar, Q, Z, n and p are as previously defined;

and pharmaceutically acceptable salts and esters thereof for use as a cathepsin inhibitor.

Dipeptide nitrile may be, for example, a compound of Formula III as defined above, wherein R₃₀ is an acyl group derived from an organic carboxylic, carbamic or sulfonic acid Compounds of Formula III are typically selective inhibitors of cathepsin B and/or L.

In one embodiment, the compounds of Formula III wherein R₃₀, R₃₂, R₃₃, R₃₄, Q, Z and n are as defined above; and wherein

(a) p is one;

(b) Y₁ is O, S, SO, SO₂, N(R₆)SO₂ or N—R₆; and

(c) X₂ is lower alkylene; or when n is zero, X₂ is also C₂-C₇-alkylene interrupted by O, S, SO, SO₂ or NR₆;

wherein R₆ is as defined above and pharmaceutically acceptable salts thereof.

Further particular embodiments relate to the compounds of Formula III wherein R₃₀, R₃₂, R₃₃, R₃₄, R₃₅, Ar, Z and Q have meaning as defined above; and wherein

(a) p is one, n is zero, and X₂ is lower alkylene or C₂-C₇-alkylene interrupted by O, S, SO, SO₂ NR₆, NR₆ SO₂, SO₂ NR₆, CONR₆ or NR₆ CO; or

(b) p is one, n is one, X₂ is lower alkylene and Y₁ is O, S, SO, SO₂, N(R)SO₂ or NR₆, SO₂ NR₆, CONR₆, NR₆ CO; or

(c) p is one, n is zero and X₂ is lower alkylene; or

(d) p is one, n is zero and X₂ is C₂-C₇-alkylene interrupted by O, S, SO, SO₂ or NR₆, SO₂ NR₆, CONR₆ or NR₂ CO; or

(e) p is zero, n is one, X₂ is lower alkylene and Y₁ is O, S, SO, SO₂, N(R₆)SO₂ or NR₆, SO₂ NR₆, CONR₆ or NR₆ CO; or

(f) p is zero, n is zero and X₂ is C₂-C₇-alkylene interrupted by O, S, SO, SO₂ or NR₆, SO₂ NR₆, CONR₆ or NR₆ CO;

and pharmaceutically acceptable salts thereof.

Preferred compounds of Formula III are those in which Z is carboxyl or carboxyl derivatized as a pharmaceutically acceptable ester.

A cathepsin inhibitor may be, for example, a dipeptide nitrile of Formula III, wherein n is zero, in particular those of Formula III′

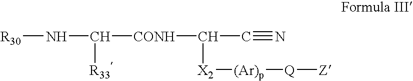

wherein:

R₃₀, X₂, Ar, Q, and p are as defined above; and wherein

R₃₃′ is carbocyclic or heterocyclic aryl-lower alkyl;

Z′ is hydroxy, acyloxy, carboxyl, carboxyl derivatized as a pharmaceutically acceptable ester or amide, or 5-tetrazolyl;

and pharmaceutically acceptable salts thereof.

A cathepsin inhibitor may be, for example, a dipeptide nitrile of Formula III′, wherein R₃₀ is carboxylic acid derived acyl; R₃₃′ is carbocyclic or heterocyclic aryl-lower alkyl; X₂ is C₁-C₅-alkylene, or X₂ is C₂-C₄-alkylene interrupted by O or S; p is one; Ar is carbocyclic arylene; Q is a direct bond or C₁-C₄-alkylene; and Z is carboxyl or carboxyl derivatized as a pharmaceutically acceptable ester; and pharmaceutically acceptable salts thereof.

In another embodiment, a cathepsin inhibitor may be, for example, a dipeptide nitrile of Formula III′, wherein R₃₀ is aroyl, R₃₃′ is carbocyclic aryl-methyl; X₂ is C₃-alkylene; or X₂ is C₂-alkylene interrupted by 0; p is one; Ar is phenylene; Q is a direct bond; and Z is carboxyl; and pharmaceutically acceptable salts thereof.

A cathepsin inhibitor may be, for example, a dipeptide nitrile of Formula III wherein n is one, in particular those of Formula III″

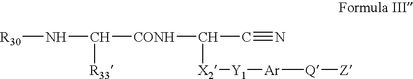

wherein

R₃₀, R₃₃′, Y₁, Ar, and Z′ are as defined above;

X₂′ is lower alkylene;

Q′ is a direct bond or lower alkylene;

and pharmaceutically acceptable salts thereof.

In one embodiment, a cathepsin inhibitor is selected from compounds of Formula III″ wherein R₃₀ is carboxylic acid derived acyl; R₃₃′ is carbocyclic or heterocyclic aryl-lower alkyl; X₂′ is C₁-C₄-alkylene; Y₁ is O or S; Ar is carbocyclic arylene; Q′ is a direct bond or C₁-C₄-alkylene; and Z′ is carboxyl or carboxyl derivatized as a pharmaceutically acceptable ester; and pharmaceutically acceptable salts thereof.

A cathepsin inhibitor may be, for example, a dipeptide nitrile of Formula III″ wherein R₃₀ is aroyl, R₃₃′ is carbocyclic aryl-methyl; X₂′ is C₂-alkylene; Y₁ is O; Ar is phenylene; Q′ is a direct bond; and Z′ is carboxyl, and pharmaceutically acceptable salts thereof.

A cathepsin inhibitor may be, for example, a dipeptide nitrile of Formula IV

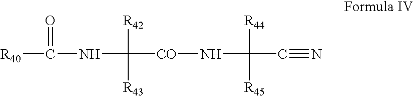

wherein

R₄₀ (is substituted phenyl or heterocyclic aryl, (mono- or di-carbocyclic or heterocyclic aryl)-lower alkyl or lower alkenyl, or heterocyclyl;

R₄₂ is hydrogen or lower alkyl;

R₄₃ is carbocyclic or heterocyclic aryl-lower alkyl;

R₄₄ and R₄₅ are independently hydrogen or lower alkyl or

R₄₄ and R₄₅ combined represent lower alkylene;

and pharmaceutically acceptable salts and esters thereof.

Other dipeptide nitriles include compounds of Formula IV wherein R₄₀ is morpholino, substituted phenyl or heterocyclic aryl; R₄₂ is hydrogen; R₄₃ is carbocyclic or heterocyclic aryl-lower alkyl; R₄₄ and R₄₅ are hydrogen or lower alkyl; or R₄₄ and R₄₅ combined represent ethylene to form a cyclopropyl ring. Also, compounds of Formula IV wherein R₄₀ is pyrazolyl or pyrazolyl substituted by 1-3 lower alkyl; R₄₂ is hydrogen; R₄₃ is carbocyclic or heterocyclic aryl-C₁-C₄-alkyl; and R₄₄ and R₄₅ are hydrogen; or R₄₄ and R₄₅ combined are ethylene.

Compounds of Formula IV are typically selective inhibitors of cathepsin L and/or S.

The compounds of Formulae I, II, III and IV, depending on the nature of substituents, possess one or more asymmetric carbon atoms. The resulting stereoisomers are encompassed by the instant invention. Preferably, however, e.g., for pharmaceutical use in accordance with the invention, the compounds of Formulae I, II, III and IV are provided in pure or substantially pure epimeric form, e.g., as compositions in which the compounds are present in a form comprising at least 90%, preferably at least 95% of a single epimer (i.e. comprising less than 10%, preferably less than 5% of other epimeric forms).

Preferred compounds of Formula I are those wherein the asymmetric carbon to which are attached R₂ and/or R₃ corresponds to that of an L-amino acid precursor and the asymmetric carbon to which is attached the cyano group also corresponds to that of an L-amino acid and is generally assigned the (S)-configuration, with the exception is cysteine having the (R)-configuration. Preferred compounds of Formula I wherein R₃ and R₄ represent hydrogen can be represented by Formulae V, V′ and V″, corresponding to preferred compounds of Formulae II, III and IV respectively.

A cathepsin inhibitor may be, for example, a dipeptide nitrile of Formula V, V′ or V″

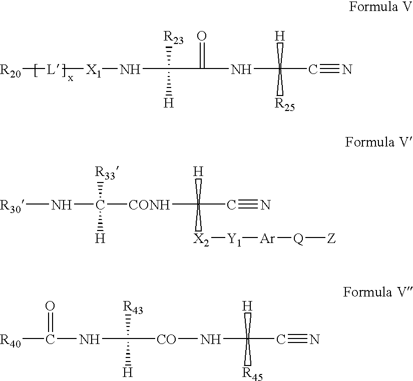

wherein the symbols are as defined above, and physiologically-acceptable and -cleavable esters or salts thereof.

The compounds of Formula I, II, II′, III, III′, III″, IV, V, V′ and V″ as defined above are therefore yet other exemplary cathepsin inhibitors.

Methods of preparing compounds by Formulas I, II, II′, III, III′, III″, IV, V, V′, and V″ above were previously described in U.S. Pat. No. 6,353,071 to which reference can be made for more information.

In view of the close relationship between the free compounds and the compounds in the form of their salts, whenever a compound is referred to in this context, a corresponding salt is also intended, provided such is possible or appropriate under the circumstances.

2) Aspartic Proteinase Inhibitors

Cathepsin inhibitor may be, for example, an aspartic proteinase inhibitor. Aspartic proteinase inhibitors are often selective inhibitors of cathepsin D. Exemplary aspartic proteinase inhibitors are listed in Table D below and were previously described. For review see Kim and Kang, “Recent developments of cathepsin inhibitors and their selectivity” Expert Opin. Ther. Patents (2002) 12(3):419-432.

TABLE D

| Compound | Name | Structure |
|---|---|---|
| 41 | sulfonamide and carboxamide derivatives | 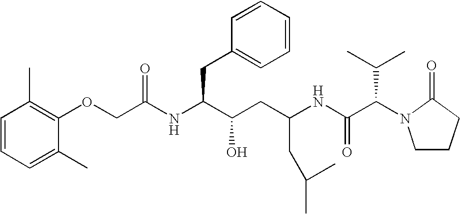 |
| 42 | modulated amyloid precursor protein and tau protein | 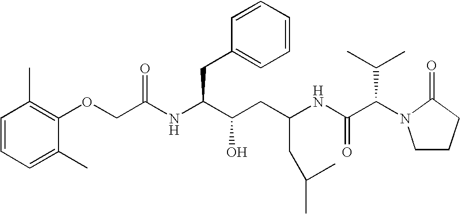 |
| 43 | hydroxypropylamide peptidomimetics | 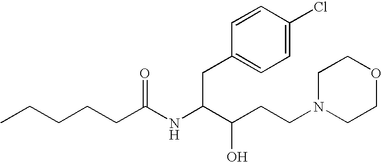 |
| 44 | hydroxystatine amide hydroxyphosphonate peptidomimetics | 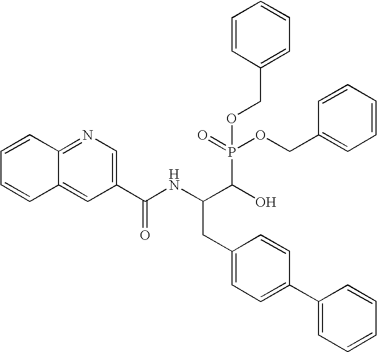 |
| 45, 46 | hydroxyamino acid amide derivatives | 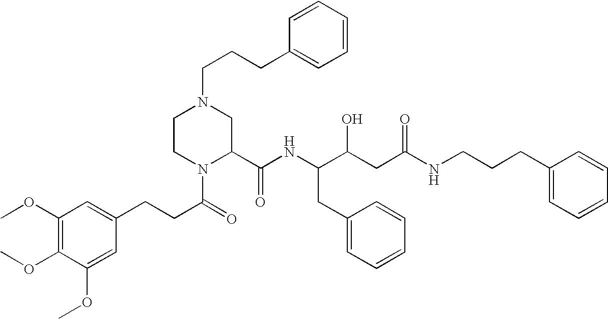 |
| | | 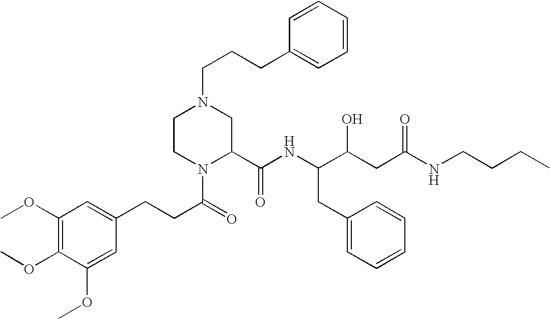 |
| 47 | peptoid compounds | 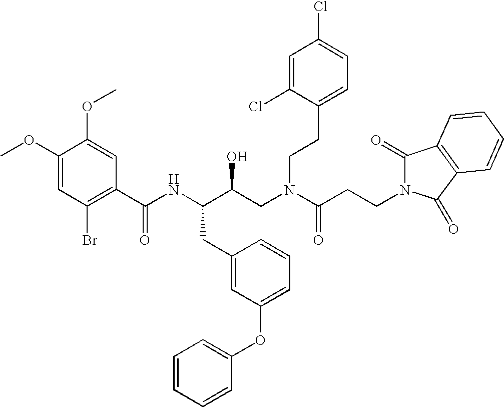 |

3) Serine Proteinase Inhibitors

Cathepsin inhibitor may be, for example, a serine proteinase inhibitor. Serine proteinase inhibitors are often selective inhibitors of cathepsin G. Exemplary aspartic serine proteinase inhibitors are listed in Table E below.

TABLE E

| Compound | Name | Structure |
|---|---|---|
| 48 | heteroaryl amidines methylamidines and guanidines | 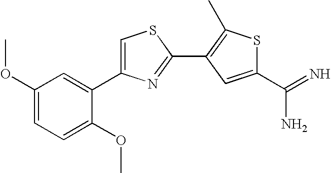 |
| 49 | 1,2,5-thiadiazolidin-3-one 1,1-dioxide derivatives | 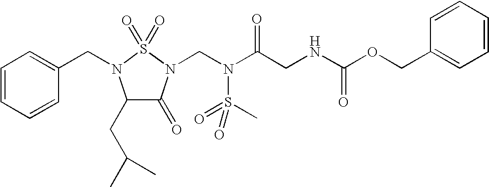 |
| 50 | transhexahydro- pyrrolo[3,2-b] pyrrolone derivatives | 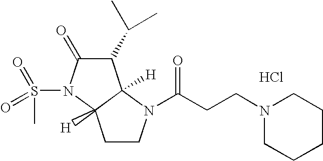 |
| 51 | pyrolopyrrolidine derivatives | 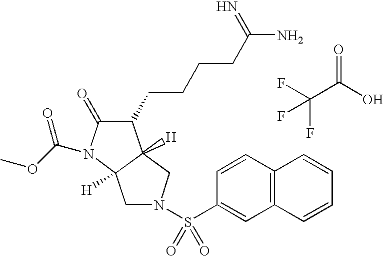 |
| 52 | furopyrrolidine derivatives | 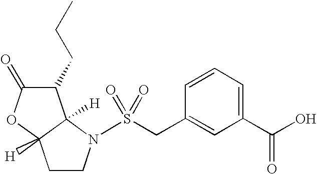 |
| 53 | anthraquinone derivatives | 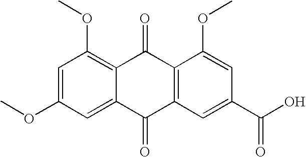 |

Cathepsin inhibitor may be, for example, a beta-phosphonic acid.

Other suitable cathepsin inhibitors, or mixtures thereof, will be known hose of ordinary skill in the art and are also included.

In one example, cathepsin inhibitor may be, for example, admixed excipients or carriers suitable for either enteral or parenteral application. In embodiment, cathepsin inhibitors may be admixed with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; and/or if desired c) disintegrants, e.g., starches, agar, alginic acid or its sodium salt, or effervescent mixtures. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 75%, preferably about 1 to 50%, of the cathepsin inhibitor.

B. Cathepsin Inhibitor Configurations

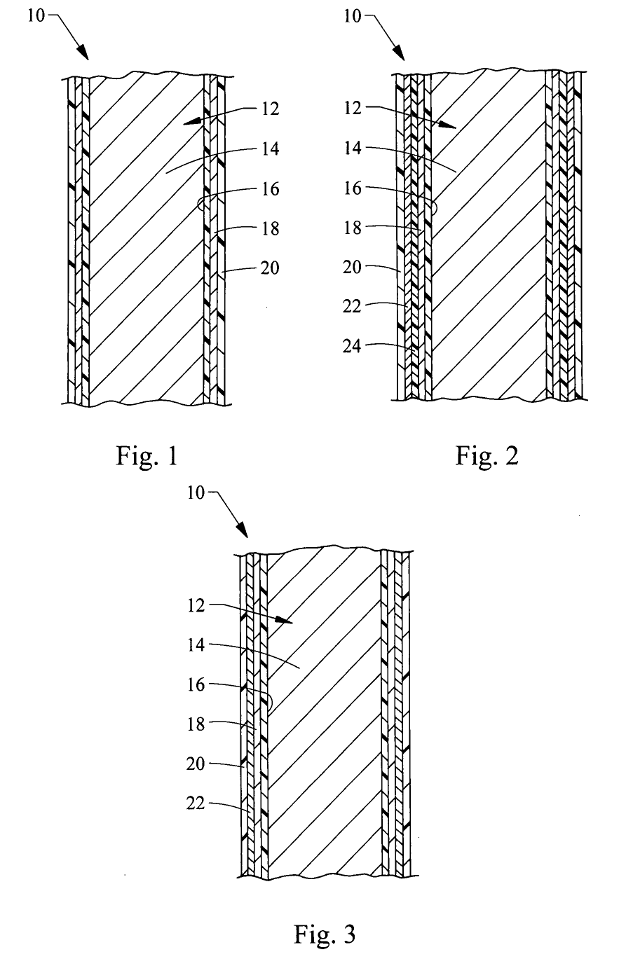

With reference to FIGS. 1-3, in one embodiment cathepsin inhibitors may be placed directly on the surface of the drug release system 12 (or on a primer layer 16, which is placed directly on the surface of the drug release system), forming a cathepsin inhibitor layer 18. One or more polymer layers may be placed over at least a portion of the cathepsin inhibitor layer 18.

In another embodiment of the invention, the cathepsin inhibitor may be incorporated with base material 14 of the drug release system 12. One or more polymer layers may be placed over at least a portion of the system 12.

In yet another embodiment, the cathepsin inhibitor may be incorporated with a primer layer 16, polymer layer 20, and/or intermediate polymer layer 24 of the device 10, as convenient or desired, and the combination may be applied to the device 10.

The term “incorporated” means that the cathepsin inhibitor is coated, adsorbed, placed, deposited, attached, impregnated, mixed, or otherwise incorporated into the device and the layers described herein by methods known in the art.

In other configurations, the cathepsin inhibitor may be linked to the surface of the drug release system without the need for a coating by means of detachable bonds and release with time, can be removed by active mechanical or chemical processes, or are in a permanently immobilized form that presents the cathepsin inhibitor at the implantation site.

In one embodiment, multiple layers of cathepsin inhibitor, or mixtures of carrier material/cathepsin inhibitor, separated by polymer layers are present to form a multicoated medical device. In certain embodiments, different cathepsin inhibitors may be present in the different layers.

A vast range of cathepsin inhibitors described above may be employed. Moreover, different cathepsin inhibitor may be included in different portions of the drug release system 12 of the device 10.

III. Drug Release System

With reference to FIG. 1, an embodiment for endoluminal medical device 10 is shown and comprises a drug release system 12 that releases a cathepsin inhibitor at a predetermined location within a lumen of a human or veterinary patient.

The drug release system may be configured as any vascular or other medical device, and can include any of variety of conventional stents, stent grafts, balloons, baskets or other device that can be deployed or permanently implanted within the vessel. Exemplary drug release systems for use with cathepsin inhibitors are described below. Moreover, the drug release system need not be an entire system, but can merely be that portion of a vascular or other device which is intended to be introduced into the patient.

The drug release system may be composed of a base material 14 suitable for the intended use of the system 12. The base material 14 is preferably biocompatible, although cytotoxic or other poisonous base materials may be employed if they are adequately isolated from the patient. Such incompatible materials may be useful in, for example, radiation treatments in which a radioactive material is positioned by catheter in or close to the specific tissues to be treated. Under most circumstances, however, the base material 14 of the release system 12 should be biocompatible.

A variety of conventional materials can be employed as the base material 14. The base material 14 may be either elastic or inelastic, depending upon the flexibility or elasticity of the polymer layers to be applied over it. The base material may be either biodegradable or non-biodegradable, and a variety of biodegradable polymers are known. Moreover, some biologic agents have sufficient strength to serve as the base material 14 of some useful release systems 12, even if not especially useful in the exemplary devices.

Accordingly, the base material 14 can include at least one of stainless steel, tantalum, titanium, nitinol, gold, platinum, inconel, iridium, silver, tungsten, cobalt, chromium, or another biocompatible metal, or alloys of any of these; carbon or carbon fiber; cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, polytetrafluoroethylene, or another biocompatible polymeric material, or mixtures or copolymers of these; polylactic acid, polyglycolic acid or copolymers thereof, a polyanhydride, polycaprolactone, polyhydroxybutyrate valerate or another biodegradable polymer, or mixtures or copolymers of these; a protein, an extracellular matrix component, collagen, fibrin or another biologic agent; a suitable mixture of any of these; and other available base materials. For example, stainless steel is particularly useful as the base material 14 when the release system 12 is configured as a stent.

Of course, when the release system 12 is composed of a radiolucent material such as polypropylene, polyethylene, or others above, a conventional radiopaque coating may and preferably should be applied to it. The radiopaque coating provides a means for identifying the location of the release system 12 by X-ray or fluoroscopy during or after its introduction into the patient's vascular system.

A. Polymer Layer

With further reference to FIG. 1, the endoluminal device may include at least one polymer layer 20.

The purpose of the polymer layer 20 may be to provide a controlled release of the cathepsin inhibitor when the device 10 is positioned at the predetermined treatment location in a patient's body. The thickness of the polymer layer 20 is chosen so as to provide such control.

Another purpose of the polymer layer may be to prevent the degradation of cathepsin inhibitor.

The polymer in the polymer layer 20 may be any material capable of releasing cathepsin inhibitor into tissue when placed in contact with the tissue. Preferably, polymer layer 20 is acceptable for at least temporary use within a human body. Polymer layer 20 is also preferably compatible with cathepsin inhibitor.

Examples of commonly used materials that may be used to form polymer layer 20 include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; and polytetrafluoroethylene (PTFE).

Polymer layer 20 may also comprise a biodegradable polymeric material, such as synthetic or natural bioabsorbable polymers. Synthetic bioabsorbable polymeric materials that can be used to form the coating layers include poly(L-lactic acid), polycaprolactone, poly(lactide-co-glycolide), poly(ethylene-vinyl acetate), poly(hydroxybutyrate-covalerate), polyd ioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) such as PEO/PLA, polyalkylene oxalates, polyphosphazenes, and other suitable biodegradable materials. The polymeric materials can be natural bioabsorbable polymers such as, but not limited to, fibrin, fibrinogen, cellulose, starch, collagen, and hyaluronic acid.

Also, biostable polymers with a relatively low chronic tissue response such as polyurethanes, silicones, and polyesters could be used and other polymers could also be used if they can be dissolved and cured or polymerized on the drug release system such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

A bioabsorbable polymer is preferred since, unlike a biostable polymer, it will not be present long after implantation to cause any adverse, chronic local response.

Polymers of polymer layer 20 may be porous, or may be made porous. Porous materials known in the art include those disclosed in U.S. Pat. No. 5,609,629 and U.S. Pat. No. 5,591,227. Typically polymers are non-porous. However, non-porous polymers may be made porous through known or developed techniques, such as extruding with CO₂, by foaming the polymeric material prior to extrusion or coating, or introducing and then removing a porogen. Non-limiting examples of porogens include salts, such as sodium bicarbonate, gelatin beads, sugar crystals, polymeric microparticles, and the like. One or more porogen may be incorporated into a polymer prior to curing or setting. The polymer may then be cured or set, and the porogen may be extracted with an appropriate solvent. Pores generated by such techniques or processes typically range in size from between about 0.01 μm to about 100 μm. The size and degree of porosity of polymer layer 20 may be controlled by the size and concentration of porogen used, the extent of mixing with gas or foaming, etc. Accordingly, the release profile of cathepsin inhibitor through or from polymer layer 20 may be controlled by varying the conditions under which pores are generated, as pore size and degree of porosity are related to release rate. For example, larger pore size, e.g., between about 1 μm and about 100 μm or between about 10 μm to 50 μm may be preferred when more rapid release of cathepsin inhibitor from polymer layer 20 is desired.

Most preferably, the polymer of the polymer layer 20 is a porous polymer. Alternatively, the polymer may be a non-porous, biodegradable polymer that releases the cathepsin inhibitor as the polymer degrades.

B. Optional Layers

As shown in FIG. 1, the device 10 may further comprise at least one primer layer 16. While the primer layer 16 can simply be any medical grade primer, the primer layer 16 is preferably composed of a polymer, and more preferably composed of the same polymer, as the polymer layer 20 described above. Examples of polymers that may be used to form the primer layer 16 were provided above in connection with the polymer layer 20.

Preferably, the primer layer 16 is less porous than the polymer layer 20, and is more preferably substantially nonporous. “Substantially nonporous” means that the primer layer 16 is sufficiently impervious to prevent any appreciable interaction between the base material 14 of the release system 12 and the blood to which the device 10 will be exposed during use. The use of primer layer 16 which is substantially nonporous would permit the use of a toxic or poisonous base material 14, as mentioned above. Even if the base material 14 of the release system 12 is biocompatible, however, it may be advantageous to isolate it from the blood by use of a substantially nonporous primer layer 16.

As shown in FIG. 2, the endoluminal medical device may include multiple layers of cathepsin inhibitor or inhibitors. For example, the device 10 may comprise a second cathepsin inhibitor layer 22 posited over the release system 12, for example, by coating. The cathepsin inhibitor of the second layer 22 can be, but need not necessarily be, different from the cathepsin inhibitor of the first cathepsin inhibitor layer 18.

The use of different cathepsin inhibitors in the layers 18 and 22 allows the device 10 to act on more than one specific cathepsin.

When the device of the present invention includes two cathepsin inhibitor layers 18 and 22, the device 10 of the present invention can further comprise at least one intermediate polymer layer 24 posited between each of the layers 18 and 22 of cathepsin inhibitor. Examples of polymers that may be used to form the intermediate polymer layer 24 were provided above in connection with the polymer layer 20.

The intermediate polymer layer 24 can give the cathepsin inhibitors in the layers 18 and 22 different release rates. Simultaneously, or alternatively, the device 10 may employ cathepsin inhibitors in the two layers 18 and 22 which are different from one another and have different solubilities. In such a case, it is advantageous and preferred to position the layer 22 containing the less soluble cathepsin inhibitor above the layer 18 containing the more soluble cathepsin inhibitor.

In one embodiment, as shown in FIG. 3, the layers 18 and 22 do not have to be separated by a polymer layer, but can instead lie directly against one another. It is still advantageous in this embodiment to position the layer containing the relatively less soluble cathepsin inhibitor above the layer containing the relatively more soluble cathepsin inhibitor.

Whether or not the intermediate polymer layer 24 is present, it is preferred that the total amount of cathepsin inhibitor posited into the drug release system 12 is in the range of about 0.01 to 10 mg/cm².

In yet another embodiment, the drug release system 12 may include a biocompatible, controlled-release matrix comprising a ligand that modulates adherence of circulating progenitor endothelial cells, which may be coated on the blood contacting surface of the medical device. Suitable ligands that modulates adherence of circulating progenitor endothelial cells, examples of suitable matrix, and methods of application of ligands to the matrix were previously described in U.S. Pat. Pub. No. 2005/0271701.

In short, the “ligand” may be a molecule that binds a cell membrane structure such as a receptor molecule on the circulating endothelial and/or progenitor cell. For example, the ligand can be an antibody, antibody fragment, small molecules such as peptides, cell adhesion molecule, basement membrane component, such as basement membrane proteins, for example, elastin, fibrin, cell adhesion molecules, and fibronectin. In an embodiment using antibodies, the antibodies recognize and bind with high affinity and specificity to progenitor endothelial cells surface antigens in the circulating blood so that the cells are immobilized on the surface of the device. The antibodies may comprise monoclonal antibodies reactive (recognize and bind) with progenitor endothelial cell surface antigens, or a progenitor or stem cell surface antigen, such as vascular endothelial growth factor receptor-1, -2 and -3 (VEGFR-1, VEGFR-2 and VEGFR-3 and VEGFR receptor family isoforms), Tie-1, Tie2, CD34, Thy-1, Thy-2, Muc-18 (CD146), CD30, stem cell antigen-1 (Sca-1), stem cell factor (SCF or c-Kit ligand), CD133 antigen, VE-cadherin, P1H12, TEK, CD31, Ang-1, Ang-2, oran antigen expressed on the surface of progenitor endothelial cells. A single type of antibody that reacts with one antigen may be used. Alternatively, a plurality of different types of antibodies directed against different progenitor endothelial cell surface antigens may be mixed together and added to the matrix. In yet another embodiment, a cocktail of monoclonal antibodies may be used to increase the rate of epithelium formation by targeting specific cell surface antigens. For example, anti-CD34 and anti-CD133 may be used in combination and attached to the surface of the matrix on a stent or graft.

C. Polymer Deposition Methods

The polymer layers may be deposited on the medical device in any known manner. Some exemplary deposition methods include coating, spraying, dipping, pouring, pumping, brushing, wiping, vacuum deposition, vapor deposition, plasma deposition, electrostatic deposition, epitaxial growth, or any other suitable method known to those skilled in the art.

Preferably, the polymer layer 20 is deposited by vapor deposition. Plasma deposition may also be useful for this purpose. Preferably, the polymer layer 20 is one that is polymerized from a vapor which is free of any solvent, catalysts or similar polymerization promoters. Also preferably, the polymer in the polymer layer 20 is one which automatically polymerizes upon condensation from the vapor phase, without the action of any added curative agent or activity such as heating, the application of visible or ultraviolet light, radiation, ultrasound, or the like. A polymer layer of a biocompatible polymer that is applied without the use of solvents, catalysts, heat or other chemicals or techniques, which would otherwise be likely to degrade or damage the cathepsin inhibitor, is preferred.

While plasma deposition and vapor phase deposition may be a preferred method for applying the various polymer coatings on the device surfaces, other techniques may be employed. For example, a polymer solution may be applied to the device and the solvent allowed to evaporate, thereby leaving on the device surface a coating of the polymer. Typically, the polymer is incorporated in a solution and sprayed onto the device until the proper thickness is achieved. Alternatively, the device may be immersed into the polymer until the proper thickness is achieved. As the polymer dries and solidifies it forms the coating layer. Methods for dip coating a medical device are disclosed in U.S. Pat. No. 6,153,252, for example.

Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the device. In either a coating applied by spraying or by immersion, multiple application steps are generally desirable to provide improved coating uniformity and improved control over the amount of polymer to be applied to the device.

Alternatively, electrostatic spray deposition method or an ultrasonic spray deposition process may be used. These methods are known in the art.

D. Surface Processing

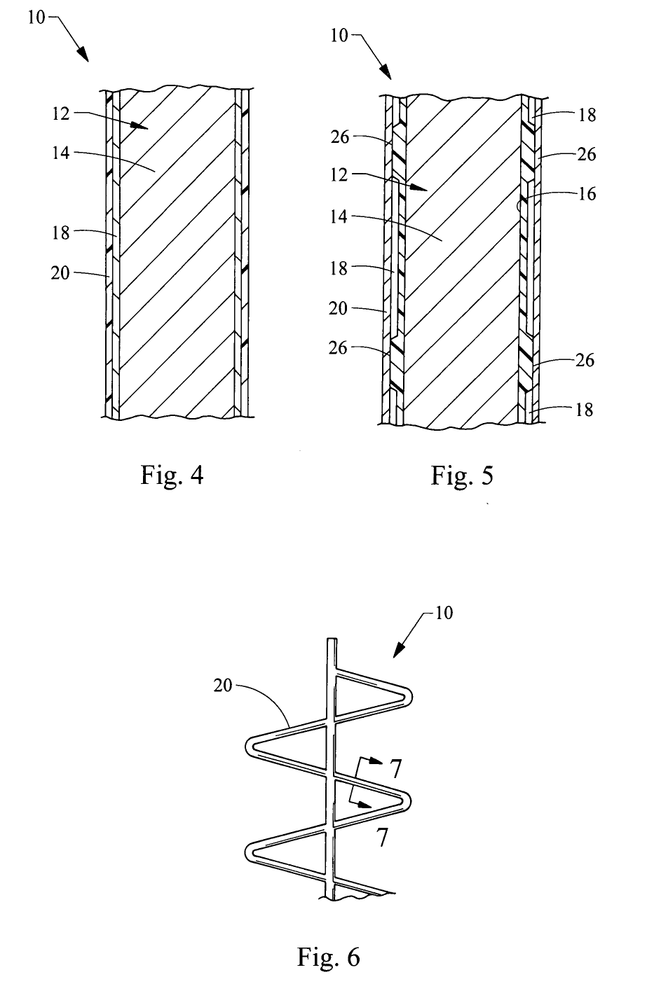

As shown in FIG. 4, the cathepsin inhibitor layer 18 may be deposited directly atop the base material 14 of the drug release system 12. In such a case, it may be highly advantageous to surface process or surface activate the base material 14, to promote the deposition and adhesion of the cathepsin inhibitor on the base material 14. Surface processing and surface activation can also selectively alter the release rate of the cathepsin inhibitor. Such processing can also be used to promote the deposition and adhesion of the primer layer 16, if present, on the base material 14. The primer layer 16 itself, or any intermediate layer 24 itself, can similarly be processed to promote the deposition and adhesion of the cathepsin inhibitor layer 18, or to further control the release rate of the cathepsin inhibitor.

Useful methods of surface processing can include any of a variety of such procedures, including: cleaning; physical modifications such as etching, drilling, cutting, or abrasion; and chemical modifications such as solvent treatment, the application of primer coatings, the application of surfactants, plasma treatment, ion bombardment and covalent bonding, which are known in the art.

The polymer layers described above may also be surface processed by any of the methods mentioned above to alter the release rate of the cathepsin inhibitor or inhibitors, and/or otherwise improve the biocompatibility of the surface of the layers. For example, the application of an overcoat of polyethylene oxide, phosphatidylcholine or a covalently bound cathepsin inhibitor, e.g., covalently attached cathepsin inhibitor to the layers 20 and/or 24 could render the surface of the layers more blood compatible. Similarly, the plasma treatment or application of a hydrogel coating to the layers 20 and/or 24 could alter their surface energies, preferably providing surface energies in the range of 20 to 30 dyne/cm, thereby rendering their surfaces more biocompatible.

Referring now to FIG. 5, an embodiment of the device 10 is there shown in which a mechanical bond or connector 26 may be provided between (a) any one of the layers 20 and 24 (not shown), and (b) any or all of the other of the layers 20 and 24 (not shown), the primer layer 16 and the base material 14. The connector 26 may reliably secure the layers 16, 20 and/or 24 to each other, and or to the base material 14. The connector 26 may lend structural integrity to the device 10, particularly after the cathepsin inhibitor layer or layers 18 and/or 22 (not shown) have been fully released into the patient. Examples of connectors as used with implantable medical devices were previously described in U.S. Pub. No. 2004/0243225.

As has been previously discussed, multiple cathepsin inhibitors, multiple layers of cathepsin inhibitors and polymer layers may be applied to the device 10 where the limiting factors become the total thickness of the endoluminal device, the adhesion of multiple layers and the like.

E. Methods of Controlling Cathepsin Inhibitor Release

Various methods of controlling the rate of release of the cathepsin inhibitors from a device are known in the art and may be used to control the release rate. For example, a coating layer may be designed according to the teachings of WO/04026361, entitled “Controllable Drug Releasing Gradient Coating for Medical Devices.”

For example, the rate of release of the cathepsin inhibitor may be controlled by placing a polymer layer 20 over the cathepsin inhibitor layer 18, which may be placed directly on the surface of the drug release system 12 or on a primer layer 16, which is placed directly on the surface of the drug release system 12, as described below.

Polymer layer 20 may be designed to control the rate at which cathepsin inhibitor is released, leached, or diffuses from or through the polymer layer 20. As used herein, “release”, “leach”, “diffuse”, “elute” and the like are used interchangeably when referring to cathepsin inhibitor with respect to polymer layer 20, base material 14, primer layer 16, or intermediate polymer layer 24 of device 10.

Varying the molecular weight of the polymer may be one way to affect the release of the cathepsin inhibitor. For example, to obtain a slower rate of release, a polymer(s) of higher molecular weight may be used. Alternatively, an amorphous polymer with higher purity may be used to obtain slower release rate. In addition, the more hydrophobic the cathepsin inhibitor, the slower the rate of release of the cathepsin inhibitor from a polymer matrix. In contrast, hydrophilic cathepsin inhibitors are released from a polymer matrix at a faster rate. Therefore, the composition of the polymer matrix can be altered according to the specific cathepsin inhibitor to be delivered in order to maintain the concentration of inhibitor required at the site for a longer period of time.

In another embodiment of the invention, the rate of release of the cathepsin inhibitor may be controlled by incorporating the cathepsin inhibitor with the polymer and coating the drug release system 12 with the resulting cathepsin inhibitor/polymer composition. The cathepsin inhibitor contained in the polymer layer 20 will then diffuse through the polymer at a rate dependent on the composition, structure, thickness, molecular weight, and purity of the polymer. Optionally, the polymer may contain pre-existing channels, through which the inhibitor may diffuse, or channels created by the release of the inhibitor or another soluble substance from the polymer.

Another technique for controlling the release of the cathepsin inhibitor may include depositing monodispersed polymeric particles, i.e., referred to as porogens, on the surface of the device 10 comprising one or more cathepsin inhibitors prior to deposition of polymer layer 20. After the polymer layer 20 is deposited and cured, the porogens may be dissolved away with the appropriate solvent, leaving a cavity or pore in the polymer layer 20 to facilitate the passage of the underlying cathepsin inhibitors.

In yet another embodiment, microencapsulated spheres, including cathepsin inhibitor, may be disposed in a polymer layer 20 on the exterior surface of the release system of the device in accordance with the techniques described in U.S. Pat. No. 6,129,705. The device includes a mechanism for radially expanding the device to cause the microencapsulated spheres to become embedded in the artery wall and thereafter to rupture to release the cathepsin inhibitor in a manner analogous to the balloon embodiment described below.

In another example, the cathepsin inhibitor may be attached to primer layer 16. Cathepsin inhibitor may be passively loaded on adsorbent primer layer 16. For example, the polymer coated stainless steel stents may be immersed in a buffered aqueous solution of cathepsin inhibitor (pH=7.2) at 37° C. Using, for example, a radio-labeled cathepsin inhibitor it may be demonstrated the approximate amount of cathepsin inhibitor was loaded per mm² device surface area. It may also be demonstrated by an in vitro flow system (16 mL/min, 4% BSA in PBS) the approximate amount of the cathepsin inhibitor remaining on the device after approximately 10 days perfusion.

Alternatively, the cathepsin inhibitor may be contained within the drug release system itself. For example, the cathepsin inhibitor may be incorporated with the base material 14 used to make the drug release system 12.

In one embodiment, the drug release system 12 may contain apertures, holes, wells, slots and the like occurring within the surface of the drug release system for containing the cathepsin inhibitor and/or coating polymer, as illustrated in FIGS. 8, 9, 10A, 10B, 10C and 10D.

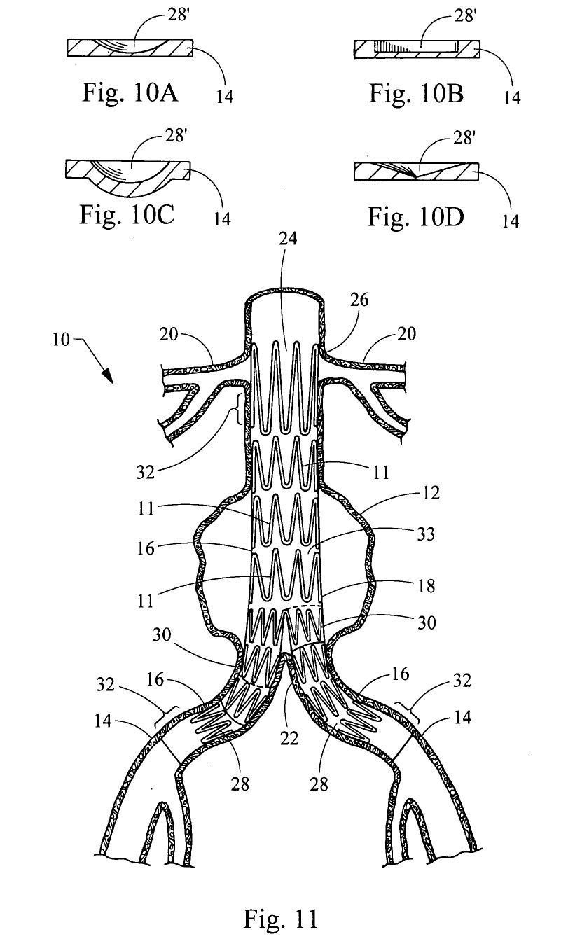

FIG. 8 shows an arm of the device of FIG. 6 wherein the arm includes holes 28 into which a cathepsin inhibitor is contained. FIG. 9 shows a section of the arm of the device along lines 9-9 of FIG. 8. Cathepsin inhibitor 18 is contained within the hole 28 where the base material 14 contains primer layer 16 and further where polymer layer 20 forms the outer most layer for the cathepsin inhibitor 18 to diffuse through. In an alternative embodiment, wells 28′ may be cut, etched or stamped into the base material 14 of the release system in which a cathepsin inhibitor 18 may be contained. This embodiment is illustrated in FIGS. 10A, 10B, 10C and 10D which are partial cross-sectional FIGS. taken along line 10-10 of FIG. 8. The wells 28′ may also be in the form of slots or grooves in the surface of the base material 14 of the release system of the device. This aspect of the invention provides the advantage of better controlling the total amount of the cathepsin inhibitor 18 to be released as well as the rate at which it is released. For example, a V-shape well 28′, as illustrated in FIG. 10D, will contain less quantity of cathepsin inhibitor 18 and release the inhibitor at geometric rate as compared to a square shaped well 28′, as illustrated in FIG. 10B, which will have a more uniform, linear release rate.

The holes, wells, slots, grooves and the like, described above, may be formed in the surface of release system of the device 10 by a variety of techniques. For example, such techniques include drilling or cutting by utilizing lasers, electron-beam machining and the like or employing photoresist procedures and etching the desired apertures.

All the cathepsin inhibitors discussed above that may be coated on the surface or otherwise incorporated with the drug release system 12 may be used to be contained within the apertures of this aspect of the invention. Likewise, layers of cathepsin inhibitors and polymer layers may be applied and built up on the exterior surfaces of the drug release system of the device as described above.

IV. Methods of Making the Device

The method of making the endoluminal device 10 as taught herein may now be understood. In its simplest form, the method comprises the steps of providing a drug release system as part of the medical device. The drug release system releases a cathepsin inhibitor at a predetermined location within a lumen of a patient. The method may further include providing at least one polymer layer configured to provide a controlled release of the cathepsin inhibitor from the device. Providing of the polymer layer is preferably by vapor deposition or plasma deposition, over the surface of the release system. The polymer layer may be composed of any suitable polymer and be of a thickness adequate to provide a controlled release of the cathepsin inhibitor. Preferably, the method further includes providing a primer layer directly on the drug release system.

Different cathepsin inhibitors may be applied to different sections or surfaces of the device.

It can be particularly convenient to apply a cathepsin inhibitor, or a mixture of the cathepsin inhibitor or inhibitors and a volatile fluid over the release system, and then remove the fluid in any suitable way, for example, by allowing it to evaporate. The cathepsin inhibitor is preferably applied in an amount as disclosed above.

The appropriate dose of cathepsin inhibitor to be included with the drug release system of the endoluminal medical device of this invention will be provided. Preferably, the cathepsin inhibitors are present in an amount effective to inhibit cathepsins once the device is deployed at a predetermined location. Preferably, the cathepsin inhibitor may be in a total amount from about 0.01 mg to about 10 mg, and more preferably from about 0.1 mg to about 4 mg of the cathepsin inhibitor per cm² of the gross surface area of the drug release system. “Gross surface area” refers to the area calculated from the gross or overall extent of the system, and not necessarily to the actual surface area of the particular shape or individual parts of the system. Preferably, about 100 μg to about 300 μg of cathepsin inhibitor per 25.4 microns of coating thickness may be contained on the device surface.

The cathepsin inhibitor may, of course, be deposited on a surface of the release system as a smooth film or as a layer of particles. Moreover, multiple but different cathepsin inhibitors may be positioned in a manner that different section and/or surfaces of the device contain the different cathepsin inhibitors. In the latter case, the particle size may affect the properties or characteristics of the device, such as the smoothness of the uppermost porous coating, the profile of the device, the surface area over which the cathepsin inhibitor layer is disposed, the release rate of the cathepsin inhibitor, the formation of bumps or irregularities in the cathepsin inhibitor layer, the uniformity and strength of adhesion of the cathepsin inhibitor layer, and other properties or characteristics. For example, in one embodiment micronized cathepsin inhibitors may have been processed to a small particle size, typically less than 10 μm in diameter. However, the cathepsin inhibitor may also be positioned as microencapsulated particles described below, dispersed in liposomes, adsorbed onto or absorbed into small carrier particles, or the like.

In still another embodiment, the cathepsin inhibitor may be incorporated with the release system in a specific geometric pattern. For example, the tips or arms of a device, such as a stent, may be free of cathepsin inhibitor, or the cathepsin inhibitor may be applied in parallel lines, particularly where two or more cathepsin inhibitors are applied to the same section of the device.

The steps of the method are preferably carried out with any of the cathepsin inhibitors, drug release systems, and base materials disclosed herein.

Exemplary drug release systems are described below.

V. Exemplary Drug Release Systems

A. Stents

In one embodiment, a cathepsin inhibitor may be released from a drug release system, such as an intraluminal stent, and delivered to a predetermined location within a lumen of a patient. Examples of drug coated stents were described in U.S. Pat. Application 2004/0243225A1.

The stent may be, for example, a Wallstent variety stent or a Gianturco-Roubin, Palmaz-Shatz, Wiktor, Strecker, Cordis, AVE Micro Stent, Igaki-Tamai, Millenium Stent, Cook-Z® Stent or Zilver Stent.

Specific examples of drug-coated stents, include BIODIVYSIO (stainless steel (SS)), S7 DRIVER (Medtronic); TRIMAXX (SS and titanium) (Medtronic); 316 SS and DURAFLEX (316L SS) (Novartus); absorbable metal magnesium stent (Biotronik); NIR stent, LIBERTE™, and EXPRESS 2™ (Boston Scientific); CoCr with holes (Conor Medsystems); SUPRA-G, V-FLEX, and ACHIEVE (Cook); expandable, flexible mesh tube made of Nitinol (Edwards Lifesciences), MULTI LINK VISION (CoCr), S-STENT (SS), MULTI-LINK TETRA, MULTI-LINK PENTA, S STENT (quadrature link), MULTI-LINK VISION delivery system/S Stent, MULTI-LINK VISION RX (CoCr) MULTI-LINK VISION (CoCr)¹³ (Guidant); IGAKI-TAMAI (Igaki Tamai); MILLENIUM STENT (Sahajanand Medical Technologies); CYPHER, SONIC, SLEEPCHASER, and BX VELOCITY (Johnson & Johnson); FLEXMASTER (ceramic) (JOMED); R-STENT (Orbus); TECNIC (Sorin Biomedica); and other suitable stents.

Some exemplary stents are disclosed in U.S. Pat. Nos. 5,292,331, 6,090,127, 5,133,732, and 4,739,762, and 5,421,955.

Referring to FIG. 1, the drug release system 12 may be a stent particularly suited for insertion into the vascular system of the patient. This stent structure can be used in other systems and sites such as the esophagus, trachea, colon, biliary ducts, urethra and ureters, subdural among others. Indeed, the system 12 can alternatively be configured as any conventional vascular or other medical device, and can include any of a variety of conventional stents or other adjuncts, such as helical wound strands, perforated cylinders, or the like. Preferably, the stent structure is designed as a stent similar to those currently used in the treatment of aneurysms, and especially aortic abdominal aneurysm. Moreover, the inserted stent need not be an entire device, but can merely be that portion of a vascular or other device which is intended to be introduced into the patient.

The stent can be biodegradable, permanently implanted, or removable. Examples of base materials that may be used for stents were described above. Stents can variable dimensions depending on the type of the stent. For example, aortic, esophageal, tracheal and colonic stents may have diameters up to about 25 mm and lengths about 100 mm or longer. For example, typical coronary artery stents are about 10 to about 60 mm in length and designed to expand to a diameter of about 2 to about 6 mm when inserted into the vascular system of the patient. Other stents may have different dimensions more suited to specific use of the stent.

The stent may be deployed according to conventional methodology, such as by an inflatable balloon catheter, by a self-deployment mechanism (after release from a catheter) (e.g., nitinol), or by other appropriate means. The stent may be formed through various methods, such as welding, laser cutting, or molding, or it may consist of filaments or fibers that are wound or braided together to form a continuous structure.

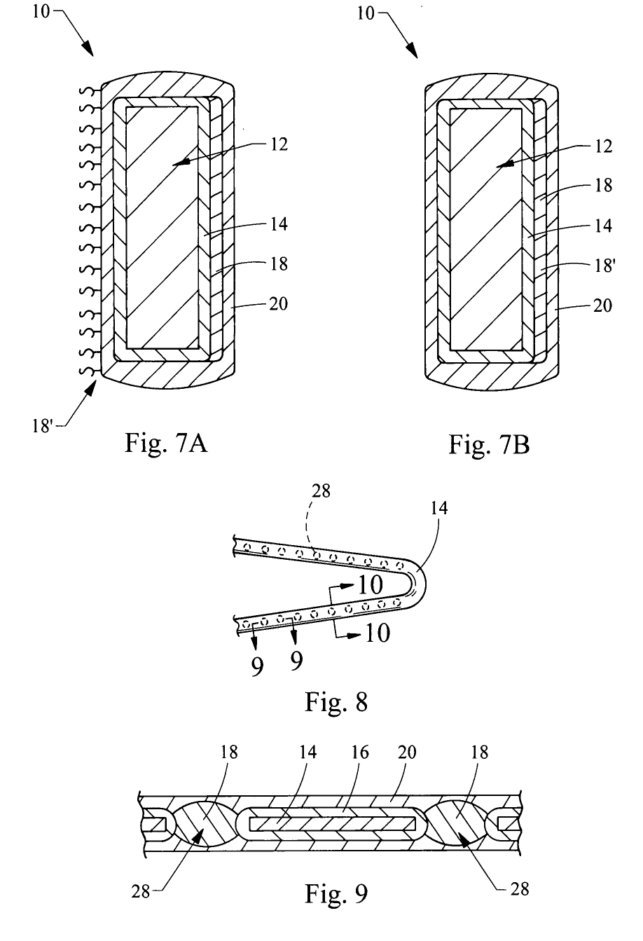

For example, FIG. 6 shows a stent 10 in its flat or planar state prior to being coiled and showing polymer layer 20 applied to its outermost surface. This polymer layer is preferably placed over the stent (drug release system). FIGS. 7A and 7B are section views along line 6-6 of FIG. 6, wherein the cathepsin inhibitor 18 may be coated on the surface of base material 14 of the stent. The cathepsin inhibitor may be a number of different cathepsin inhibitors or combination of the cathepsin inhibitors described above. For example, the stent may be placed in the body of a patient near an aneurysm to deliver a cathepsin inhibitor directly to the aneurysm. A polymer layer 20 may be posited over the stent to provide a smoother surface as well as a more controlled release of the cathepsin inhibitor 18. As further illustrated in FIG. 7A, the opposite surface of the device may have, for example, different cathepsin inhibitor 18′ covalently bonded to polymer layer 20. It is pointed out, as has been discussed herein, a third cathepsin inhibitor may be posited (not shown) on the surface of base material 14.

B. Stent Grafts

In one embodiment, the stent further is a stent graft and comprises a tubular graft material supported by the stent. Accordingly, a cathepsin inhibitor may be released from the stent graft and delivered to a predetermined location within a lumen of a patient.

A known stent graft that may be suitable for use with the cathepsin inhibitors is the Zenith AAA™ stent graft sold by William A. Cook Australia Pty., Brisbane, Australia and Cook, Inc., Bloomington, Ind.

The tubular graft material may comprise a textile fabric, a polymer, biomaterial, or a composite thereof, in which the cathepsin inhibitor is incorporated into the material (e.g., polymer, biomatrix) or otherwise coated on, bonded to, or impregnated thereinto. Additionally, the cathepsin inhibitor-loaded graft material may be made of biodegradable fiber material that does not break into larger pieces. An example of a suitable fiber material is one that comprises at least two layers that degrade or are resorbed at different rates. Examples of various graft materials and methods of incorporating the cathepsin inhibitors into the graft material are described below.

A graft material may be a biocompatible textile. The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

Examples of biocompatible materials from which textile can be formed include polyesters, such as poly(ethylene terephthalate); fluorinated polymers, such as polytetrafluoroethylene (PTFE) and fibers of expanded PTFE; and polyurethanes. In addition, materials that are not inherently biocompatible may be subjected to surface modifications in order to render the materials biocompatible. Examples of surface modifications include graft polymerization of biocompatible polymers from the material surface, coating of the surface with a crosslinked biocompatible polymer, chemical modification with biocompatible functional groups, and immobilization of a compatibilizing agent such as heparin or other substances. Thus, any fibrous material may be used to form a textile graft material, provided the final textile is biocompatible. Textile materials that can be formed into fibers suitable for making textiles include polyethylene, polypropylene, polyaramids, polyacrylonitrile, nylons and cellulose, in addition to polyesters, fluorinated polymers, and polyurethanes as listed above. Preferably the textile is made of one or more polymers that do not require treatment or modification to be biocompatible. More preferably, the textile is made of a biocompatible polyester. One example of biocompatible polyester includes Dacron™ (DUPONT, Wilmington, Del.).

One example of suitable stent graft is disclosed in PCT Publication No. WO 98/53761, in which the stent graft includes a sleeve or tube of biocompatible graft material such as Dacron™.

Textile graft material may be from woven (including knitted) textiles or nonwoven textiles. Nonwoven textiles are fibrous webs that are held together through bonding of the individual fibers or filaments. The bonding can be accomplished through thermal or chemical treatments or through mechanically entangling the fibers or filaments. Because nonwovens are not subjected to weaving or knitting, the fibers can be used in a crude form without being converted into a yarn structure. Woven textiles are fibrous webs that have been formed by knitting or weaving. The woven textile structure may be any kind of weave including, for example, a plain weave, a herringbone weave, a satin weave, or a basket weave.

In one example of woven textiles, knitted textiles include weft knit and warp knit fiber arrays. Weft knit fabric structures (including double-knit structures) utilize interlocked fiber loops in a filling-wise, or weft, direction, while warp knit structures utilize fabric loops interlocked in a length wise, or warp, direction. Weft knit structures generally are more elastic than warp knit structures, but the resiliency of warp knit fabrics is satisfactory to provide a substantial degree of elasticity, or resiliency, to the fabric structure without substantially relying on tensile fiber elongation for such elasticity. Weft knit fabrics generally have two dimensional elasticity (or stretch), while warp knit fabrics generally have unidirectional (width wise) elasticity. The different elasticity properties of the various knit or woven structures may be beneficially adapted to the functional requirement of the particular device application. In some cases, where little elasticity is desired, the fabric may be woven to minimize in plane elasticity but yet provide flexibility. Commercially available woven and knitted fabrics of medical grade Dacron fibers including, single and double velour graft fabrics, stretch Dacron graft fabric and Dacron mesh fabrics, provided the fibers that have suitably small diameter and other properties to provide graft material of an implantable device of the type taught herein. For smaller vascular graft applications (less than 6 mm diameter), and for other applications for which suitable substrates of desired structure are not commercially available, special manufacture may be necessary.

Woven fabrics may have any desirable shape, size, form and configuration. For example, the fibers of a woven fabric may be filled or unfilled. Examples of how the basic unfilled fibers may be manufactured and purchased are indicated in U.S. Pat. No. 3,772,137. Fibers similar to those described are currently being manufactured by the DuPont Company from polyethylene terephthalate (often known as “DACRON™” when manufactured by DuPont), and by other companies from various substances. Certain physical parameters may be used to characterize the textile fibers. The fibers may have a tensile strength of at least about 20,000 psi and a tensile modulus of at least about 2×10⁶ psi. Preferably, the textile is made of medical grade synthetic polymeric materials. The fibers of the textile may also have a high degree of axial orientation. The fibers may be of diameter from about 1 micron to about 5 millimeters. The denier of the textile may be from 0.5 denier per filament to 5 denier per filament. Preferably the interstices between the fibers of the textile comprise a maximum interstices spacing from about 1 micron to about 400 microns. More preferably, the interstices between the fibers of the textile comprise a maximum interstices spacing from about 1 micron to about 100 microns. Most preferably, the interstices between the fibers of the textile comprise a maximum interstices spacing from about 1 micron to about 10 microns.

Preferred textiles include those formed from polyethylene terephthalate and PTFE. These materials are inexpensive, easy to handle, have good physical characteristics and are suitable for clinical application.

In textile devices, the fibers provide a flexible array in sheet or tubular form so that the graft material is provided with a predetermined high degree of flexibility of the graft material which also has beneficial biologically compatible properties of extracellular collagen matrix. Furthermore, a high degree of elasticity may be provided through bending of the fibers of the array rather than through substantial tensile elongation of the fibers.

Preferred textile graft materials are made of woven polyester having a twill weave and a porosity of about 350 mL/min/cm² (available from VASCUTEK® Ltd., Renfrewshire, Scotland, UK).

The graft material may be a biocompatible polymer material. Preferably, the polymer material is porous.

Examples of biocompatible polymers from which polymer graft materials can be formed include polyesters, such as poly(ethylene terephthalate), polylactide, polyglycolide and copolymers thereof; fluorinated polymers, such as polytetrafluoroethylene (PTFE), expanded PTFE and poly(vinylidene fluoride); polysiloxanes, including polydimethyl siloxane; and polyurethanes, including polyetherurethanes, polyurethane ureas, polyetherurethane ureas, polyurethanes containing carbonate linkages and polyurethanes containing siloxane segments. In addition, materials that are not inherently biocompatible may be subjected to surface modifications in order to render the materials biocompatible. Examples of surface modifications include graft polymerization of biocompatible polymers from the material surface, coating of the surface with a crosslinked biocompatible polymer, chemical modification with biocompatible functional groups, and immobilization of a compatibilizing agent such as heparin or other substances. Other suitable polymers include polyolefins, polyacrylonitrile, nylons, polyaramids and polysulfones, in addition to polyesters, fluorinated polymers, polysiloxanes and polyurethanes as listed above. Preferably the graft material is made of one or more polymers that do not require treatment or modification to be biocompatible. More preferably, the graft material includes a biocompatible polyurethane. Examples of biocompatible polyurethanes include THORALON (THORATEC, Pleasanton, Calif.), BIOSPAN, BIONATE, ELASTHANE, PURSIL and CARBOSIL (POLYMER TECHNOLOGY GROUP, Berkeley, Calif.).

Preferably the polymer graft material contains the polyurethane THORALON. As described in U.S. Pub. No. 2002/0065552 A1, incorporated herein by reference, THORALON is a polyetherurethane urea blended with a siloxane-containing surface modifying additive. Specifically, the polymer is a mixture of base polymer BPS-215 and an additive SMA-300. The concentration of additive may be in the range of 0.5% to 5% by weight of the base polymer. The BPS-215 component (THORATEC) is a segmented polyether urethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED). The SMA-300 component (THORATEC) is a polyurethane comprising polydimethylsiloxane as a soft segment and the reaction product of MDI and 1,4-butanediol as a hard segment. A process for synthesizing SMA-300 is described, for example, in U.S. Pat. Nos. 4,861,830 and 4,675,361. A polymer graft material can be formed from these two components by dissolving the base polymer and additive in a solvent such as dimethylacetamide (DMAC) and solidifying the mixture by solvent casting or by coagulation in a liquid that is a non-solvent for the base polymer and additive.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON may be useful in larger vessels, such as the abdominal aorta, where elasticity and compliance is beneficial.

In addition to THORALON, other polyurethane ureas may be used as a graft material. For example, the BPS-215 component with a MDI/PTMO mole ratio ranging from about 1.0 to about 2.5 may be used.

In addition to polyurethane ureas, other polyurethanes, preferably those having a chain extended with diols, may be used as a graft material. Polyurethanes modified with cationic, anionic and aliphatic side chains may also be used. See, for example, U.S. Pat. No. 5,017,664. Polyurethanes may need to be dissolved in solvents such as dimethyl formamide, tetrahydrofuran, dimethyacetamide, dimethyl sulfoxide, or mixtures thereof.

In addition, the polyurethanes may also be end-capped with surface active end groups, such as, polydimethylsiloxane, fluoropolymers, polyolefin, polyethylene oxide, or other suitable groups. See, for example the surface active end groups disclosed in U.S. Pat. No. 5,589,563.

In one embodiment, the graft material may contain a polyurethane having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pub. No. 2002/0187288 A1.

The graft material may contain polytetrafluoroethylene or expanded polytetraffluoroethylene (ePTFE). The structure of ePTFE can be characterized as containing nodes connected by fibrils. The structure of ePTFE is disclosed, for example, in U.S. Pat. Nos. 6,547,815; 5,980,799; and 3,953,566.

Polymers can be processed to form porous polymer graft materials using standard processing methods, including solvent-based processes such as casting, spraying and dipping, and melt extrusion processes. Extractable pore forming agents can be used during processing to produce porous polymer graft material. Examples of the particulate used to form the pores in the first coat may be a salt, including, but not limited to, sodium chloride (NaCl), sodium bicarbonate (NaHCO₃), Na₂CO₃, MgCl₂, CaCO₃, calcium fluoride (CaF₂), magnesium sulfate (MgSO₄), CaCl₂, AgNO₃ or any water soluble salt. However, other suspended particulate materials may be used. These include, but are not limited to, sugars, polyvinyl alcohol, cellulose, gelatin or polyvinyl pyrolidone. Preferably, the particulate is sodium chloride; more preferably, the particulate is a sugar. Preferably, the size of the particles ranges from about 5 to about 50 microns.

The amount of pore forming agent relative to the polymer may be from about 20 percent by weight (wt %) to about 90 wt %, and from about 40 wt % to about 70 wt %. These sizes and amounts of pore forming agents can provide for a high degree of porosity following extraction of the pore forming agent. The porosity can be from about 20 wt % to about 90 wt %, and from about 40 wt % to about 70 wt % of the final product.

Porous polymer may be in the form of a microporous, open-celled structure in which the pores are substantially interconnected. Microporous structures can be formed by extrusion of a mixture of polymer and one or more blowing agents. Microcellular polymeric foams can be produced by exposing the polymer to super-critical CO₂ under high temperature and pressure to saturate the polymer with the super-critical CO₂, and then cooling the polymer. Microcellular foams can be produced as described, for example, in U.S. Pat. Nos. 4,473,665 and 5,160,674, which are incorporated herein by reference in their entirety. The foaming process can be carried out on extruded polymer tube by first dissolving an inert gas such as nitrogen or CO₂ under pressure into the polymer, and then forming microvoids by quickly decreasing the solubility of the gas in the polymer by changing the pressure or temperature, thus inducing thermodynamic instability. Examples of microporous polymeric structures are disclosed, for example, in U.S. Pat. No. 6,702,849 B1.

Porous THORALON can be formed by mixing the polyetherurethane urea, the surface modifying additive and a particulate substance in a solvent. Preferably the particulate is insoluble in the solvent, and the particulate may be any of a variety of different particulates or pore forming agents described above. For example, the solvent may be DMAC, and the particulate may be an inorganic salt. The composition can contain from about 5 wt % to about 40 wt % polymer, and different levels of polymer within the range can be used to fine tune the viscosity needed for a given process. The composition can contain less than 5 wt % polymer for some spray application embodiments. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water for the extraction, for example water at a temperature of about 60° C. Alternatively, a lubricant, such as Liqui-NOX® detergent may be used. The resulting void-to-volume ratio can be substantially equal to the ratio of salt volume to the volume of the polymer plus the salt. The resulting pore diameter can also be substantially equal to the diameter of the salt grains.

The porous polymer graft material may have a void-to-volume ratio from about 0.40 to about 0.90. Preferably the void-to-volume ratio is from about 0.65 to about 0.80. Void-to-volume ratio is defined as the volume of the pores divided by the total volume of the polymeric layer including the volume of the pores. The void-to-volume ratio can be measured using the protocol described in AAMI (Association for the Advancement of Medical Instrumentation) VP20-1994, Cardiovascular Implants—Vascular Prosthesis section 8.2.1.2, Method for Gravimetric Determination of Porosity. The pores in the polymer can have an average pore diameter from about 1 micron to about 400 microns. Preferably the average pore diameter is from about 1 micron to about 100 microns, and more preferably is from about 1 micron to about 10 microns. The average pore diameter is measured based on images from a scanning electron microscope (SEM). Formation of porous THORALON is described, for example, in U.S. Pub. Nos. 2003/0114917 A1 and 2003/0149471 A1.

The graft material may be a biomaterial, such as naturally occurring extracellular matrix (ECM), or naturally occurring biopolymers. ECM is preferred.

ECM is the noncellular part of a tissue and consists of protein and carbohydrate structures secreted by the resident cells. ECM serves as a structural element in tissues. The extracellular matrix can be isolated and treated in a variety of ways.

The ECM for use in the endoluminal device can be selected from a variety of commercially available matrices including collagen matrices, or can be prepared from a wide variety of natural sources of collagen. Examples of these naturally occurring ECMs include submucosa, dura mater, pericardium, serosa, peritoneum, acellular dermis, cadaveric fascia, the bladder acellular matrix graft, amniotic membrane (for review see Hodde J., Tissue Engineering 8(2):295-308 (2002)), and basement membrane tissues. Suitable submucosa tissues include, for instance, intestinal submucosa, stomach submucosa, urinary bladder submucosa, and uterine submucosa. In addition, collagen-based extracellular matrices derived from renal capsules of warm blooded vertebrates may be selected for use in preparing the graft materials of this invention. The extracellular matrices derived from renal capsules of warm blooded vertebrates were described in WO 03/02165. Juvenile submucosa tissue may also be used. Juvenile submucosal tissue was described in WO 04/22107.

Another type of ECM, isolated from liver basement membrane, is described in U.S. Pat. No. 6,379,710. ECM may also be isolated from pericardium, as described in U.S. Pat. No. 4,502,159.

These ECMs may be derived generally from warm-blooded vertebrates, more preferably mammals such as porcine, bovine, or ovine mammals. Human donor tissues may also be used. These ECMs may be used in any suitable form, including their use as layers.

In addition to xenogenic biomaterials, autologous tissue can be harvested as well. Additionally elastin or elastin-like polypeptides (ELPs) and the like offer potential as a biologically active ECM. Another alternative would be to use allographs such as harvested native valve tissue. Such tissue is commercially available in a cryopreserved state.

The graft material may be, for example, submucosa. “Tela submucosa” or “submucosa” refers to a layer of collagen-containing connective tissue occurring under the mucosa in most parts of the alimentary, respiratory, urinary and genital tracts of animals. Submucosa is a preferred source of ECM. Purified submucosa, a preferred type of ECM, has been previously described in U.S. Pat. Nos. 6,206,931, 6,358,284 and 6,666,892 as a bio-compatible material that enhances the repair of damaged or diseased host tissues. U.S. Pat. Nos. 6,206,931, 6,358,284 and 6,666,892. The submucosa may be derived from intestine. The mucosa can also be derived from vertebrate liver tissue as described in WIPO Publication, WO 98/25637, based on PCT application PCT/US97/22727; from gastric mucosa as described in WIPO Publication, WO 98/26291, based on PCT application PCT/US97/22729; from stomach mucosa as described in WIPO Publication, WO 98/25636, based on PCT application PCT/US97/23010; or from urinary bladder mucosa as described in U.S. Pat. No. 5,554,389.

The submucosa is preferably derived from the intestines, more preferably the small intestine, of a warm blooded vertebrate; i.e., small intestine submucosa (SIS). SIS is commercially available from Cook Biotech, West Lafayette, Ind. Preferred intestine submucosal tissue typically includes the tunica submucosa delaminated from both the tunica muscularis and at least the luminal portions of the tunica mucosa. In one example the submucosal tissue includes the tunica submucosa and basilar portions of the tunica mucosa including the lamina muscularis mucosa and the stratum compactum. The preparation of intestinal submucosa is described in U.S. Pat. No. 4,902,508, and the preparation of tela submucosa is described in U.S. patent application Ser. No. 08/916,490. The preparation of submucosa is also described in U.S. Pat. No. 5,733,337 and in 17 Nature Biotechnology 1083 (November 1999); and WIPO Publication WO 98/22158, dated 28 May 1998, which is the published application of PCT/US97/14855. Also, a method for obtaining a highly pure, delaminated tela submucosa collagen matrix in a substantially sterile state was previously described in U.S. Pub. No. 20040180042.

The stripping of the tela submucosa source is preferably carried out by utilizing a disinfected or sterile casing machine, to produce a tela submucosa which is substantially sterile and which has been minimally processed. A suitable casing machine is the Model 3-U-400 Stridhs Universal Machine for Hog Casing, commercially available from the AB Stridhs Maskiner, Gotoborg, Sweden. As a result of this process, the measured bioburden levels may be minimal or substantially zero. Other means for delaminating the tela submucosa source can be employed, including, for example, delaminating by hand.

In this method, a segment of vertebrate intestine, preferably harvested from porcine, ovine or bovine species, may first be subjected to gentle abrasion using a longitudinal wiping motion to remove both the outer layers, identified as the tunica serosa and the tunica muscularis, and the innermost layer, i.e., the luminal portions of the tunica mucosa. The submucosal tissue is rinsed with water or saline, optionally sterilized, and can be stored in a hydrated or dehydrated state. Delamination of the tunica submucosa from both the tunica muscularis and at least the luminal portions of the tunica mucosa and rinsing of the submucosa provide an acellular matrix designated as submucosal tissue. The use and manipulation of such material for the formation of ligament and tendon grafts and the use more generally of such submucosal tissue constructs for inducing growth of endogenous connective tissues is described and claimed in U.S. Pat. No. 5,281,422.

Following delamination, submucosa may be sterilized using any conventional sterilization technique including propylene oxide or ethylene oxide treatment and gas plasma sterilization. Sterilization techniques which do not adversely affect the mechanical strength, structure, and biotropic properties of the purified submucosa are preferred. Preferred sterilization techniques also include exposing the submucosa to ethylene oxide treatment or gas plasma sterilization. Typically, the purified submucosa is subjected to two or more sterilization processes. After the purified submucosa is sterilized, for example by chemical treatment, the matrix structure may be wrapped in a plastic or foil wrap and sterilized again using electron beam or gamma irradiation sterilization techniques.

Preferred submucosa graft material may also be characterized by the low contaminant levels set forth in Table 1 below. The contaminant levels in Table 1 may be found individually or in any combination in a given ECM sample. The abbreviations in Table 1 are as follows: CFU/g=colony forming units per gram; PFU/g=plaque forming units per gram; μg/mg=micrograms per milligram; ppm/kg=parts per million per kilogram.

TABLE 1

| | FIRST | | SECOND | | THIRD | |
|---|---|---|---|---|---|---|
| | PREFERRED | | PREFERRED | | PREFERRED | |
| | LEVEL | | LEVEL | | LEVEL | |
| ENDOTOXIN | <12 | EU/g | <10 | EU/g | <5 | EU/g |
| BIOBURDEN | <2 | CFU/g | <1 | CFU/g | <0.5 | CFU/g |
| FUNGUS | <2 | CFU/g | <1 | CFU/g | <0.5 | CFU/g |
| NUCLEIC | <10 | μg/mg | <5 | μg/mg | <2 | μg/mg |
| ACID | | | | | | |
| VIRUS | <500 | PFU/g | <50 | PFU/g | <5 | PFU/g |
| PROCESS- | <100,000 | ppm/kg | <1,000 | ppm/kg | <100 | ppm/kg |
| ING AGENT | | | | | | |

Purified submucosa may be further processed in a number of ways to provide ECM suitable for the graft material to use in the device of this invention.

It is also possible to form large surface area constructs by combining two or more submucosa sections using techniques described in U.S. Pat. Nos. 2,127,903 and 5,711,969. Thus, a plurality of submucosa strips can be fused to one another, for example by compressing overlapping areas of the strips under dehydrating conditions, to form an overall planar construct having a surface area greater than that of any one planar surface of the individual strips used to form the construct.

Variations of the above-described processing procedures may be used to produce submucosa that may be used as the graft material of a stent graft. For example, the source tissue for the submucosa, e.g., stomach, whole intestine, cow uterus and the like, can be partially delaminated, treated with a disinfecting or sterilizing agent followed by complete delamination of the submucosa. Illustratively, attached mesentery layers, and/or serosa layers of whole intestine can be removed prior to treatment with the disinfecting agent, followed by delamination of remaining attached tissues from the tela submucosa. These steps may or may not be followed by additional disinfection steps, e.g., enzymatic purification and/or nucleic acid removal. Alternatively, the submucosa source can be minimally treated with a disinfecting or other such agent, the submucosa delaminated from the tunica muscularis and tunica mucosa, followed by a complete disinfection treatment to attain the desired contaminant level(s).

The purified submucosa can be conditioned, as described in U.S. patent application Ser. No. 08/916,490, to alter the viscoelastic properties of the purified submucosa. The purified submucosa may be conditioned by stretching, chemically treating, enzymatically treating or exposing the matrix structure to other environmental factors. In one embodiment, the strips of purified tela submucosa may be conditioned by stretching in a longitudinal and/or lateral direction to a strain of no more than 20%. Strain is the percentage increase in the length of the material after loading.

In another embodiment, the purified submucosa may be conditioned by stretching the material longitudinally to a length longer than the length of the purified submucosa from which the ECM was formed. The conditioning process and other relevant processes are described in U.S. Pat. No. 6,358,284.

Additionally, the cathepsin-loaded graft material can be made of biodegradable fiber material that does not break into larger pieces. An example of a suitable fiber material is one that comprises at least two layers that degrade or are reabsorbed at different rates.

In addition to delivering cathepsin inhibitors, the ECM graft material may serve as a matrix for, promote and/or induce the growth of endogenous tissue and undergo a process of bioremodeling. Common events related to this bioremodeling process may include widespread and rapid neovascularization, proliferation of granulation mesenchymal cells, biodegradation/resorption of implanted purified intestine submucosa material, and lack of immune reaction. Cathepsin inhibitors may advance the healing process by producing a desired biological effect in vivo (e.g., reduce elastin breakdown leading to dialation of the vessels and aneurysm).

Cathepsin inhibitors, for example, may be incorporated into or covalently attached to the graft material during the process of preparing of the graft material. For example, the cathepsin inhibitor may be incorporated (for example, by impregnation) into the graft material. In one embodiment of this invention, cathepsin inhibitors may be first mixed with the fluidized biomaterial, such as fluidized SIS to form a substantially homogenous graft material, including the cathepsin inhibitor. The fluidized mixture of biomaterial and the cathepsin inhibitor(s) is then allowed to dry before applying it to a device. The preparation of fluidized forms of intestine submucosa is described in U.S. Pat. Nos. 5,275,826, 5,516,533, and 6,264,992.

Alternatively, cathepsin inhibitors may be added to the graft material after preparation of the graft material, e.g., by soaking, spraying, painting, impregnating, or otherwise applying the cathepsin inhibitor to the graft material by methods known in the art.

In yet another embodiment, holes, wells, slots and the like may be introduced into the graft material of the stent graft to hold the cathepsin inhibitor.

In another example, the cathepsin inhibitor may be incorporated into a polymer coating placed over the graft material.

In one embodiment, a graft material, such as polymer graft material or the ECM graft material, may be populated with endothelial cells or precursors thereto on the interior lumen side of the graft, when the graft is in a preferred tubular form. The endothelial cells, or precursors thereto, may be derived from any suitable source of endothelial cells including vascular endothelial cells from arterial or venous tissues. In yet another embodiment, at least one additional exogenous cell population may be included on the graft material. The additional exogenous cell population may be any cell population adding to the functional characteristics and durability of the graft material. Preferably, the exogenous cell population includes muscle cells or precursors to muscle cells. Smooth muscle cells and precursor cells are more preferred. Suitable muscle cells and precursor cells are disclosed for example in WO 178754. The exogenous cells may also include fibroblasts, or precursors thereto. In one embodiment, endothelial cells, preferably vascular endothelial cells, fibroblasts, and smooth muscle cells (or precursors to any of these) may be seeded onto the graft material either as separate cell layers or admixed together. Cell-seeded extracellular matrix grafts were previously described in U.S. Pat. Pub. No. 2005/0202058 A1.

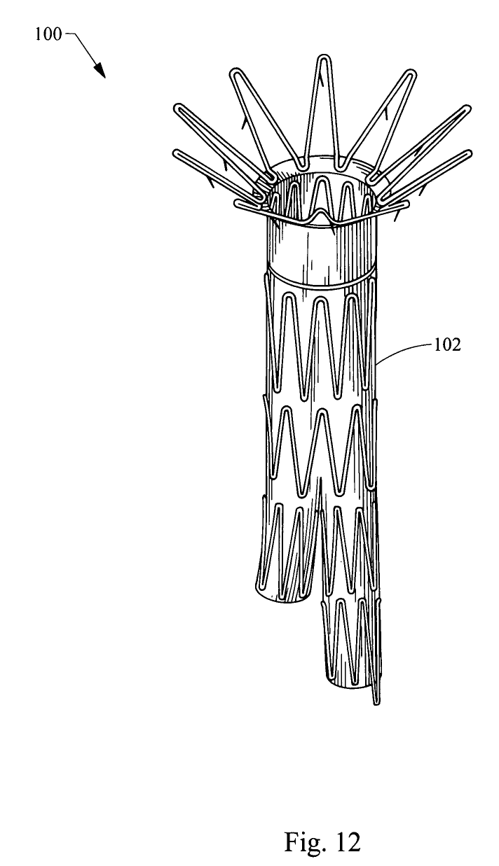

FIG. 11 shows an example of a modular bifurcated stent graft 10 deployed within an aneurysmal aorta 12 and both iliac arteries 14. The cathepsin inhibitor is deposited within the graft material 33. The prosthetic modules 16 that make up the stent graft 10 are generally tubular, so that the fluid can flow through the stent graft 10, and are preferably made of biocompatible polyurethane, polysiloxane, polyester, fluorinated polymer; or a textile, such as poly(ethylene terephthalate) or similar materials described above. The main body 18 extends from the renal arteries 20 to near the bifurcation 22. Multiple Z-stents 11 are sutured along the length of the stent graft 10. A suprarenal fixation stent 24 anchors the main body 18 to the healthier, preferably non-aneurysmal tissue 26 near the renal arteries. Two iliac extension modules 28 extend from the iliac limbs 30.

The stent graft 10 will preferably achieve a blood-tight seal at the contact regions 32 on both ends of the aneurysm 12, so that the aneurysm 12 will be excluded. In the particular embodiment shown in FIG. 2, the stent graft 10 contacts the vascular tissue below the renal arteries 20, around the bifurcation 22 and at the iliac limbs 30 and extensions 28. In this embodiment, a seal is preferably achieved that will help exclude the entire aneurysmal region and, as a result, the hemodynamic pressures within the aneurysm 12 may be reduced. The cathepsin inhibitor released from the stent graft will work to stop and/or reverse the progression of endovascular disease, preventing further weakening and dialation of vessel wall.

FIG. 12 shows another example of a modular bifurcated stent graft 100. This figure shows a three-piece modular bifurcated stent graft 100 also designed for deployment into an aorta. Cathepsin inhibitors are impregnated into the graft material 102.

C. Balloons

In yet another embodiment, the endoluminal device may comprise a drug release system and a delivery system for delivering the device. The drug release system may be integrated with the delivery system. The delivery system may include a structure such as catheter with a balloon and delivered to a predetermined location within a lumen of a patient Examples of balloons used for drug delivery were described in U.S. Pub. No. 2004/0073190 A1.

The drug release system could be incorporated in the delivery system alone, in the medical device alone, or in both. In the latter event, the delivery system and device could include the same or different cathepsin inhibitors or inhibitor functions.

Usually, the catheters will be intravascularly introduced, typically through a cut down or through the well-known Seldinger technique. The catheters will comprise a catheter body, typically which is introducable over a steerable guidewire. The drug release system at its distal end, will include a balloon to perform the desired cathepsin inhibitor delivery and release.

In one embodiment, a cathepsin inhibitor may be coated or otherwise placed on the outer surface of a balloon.

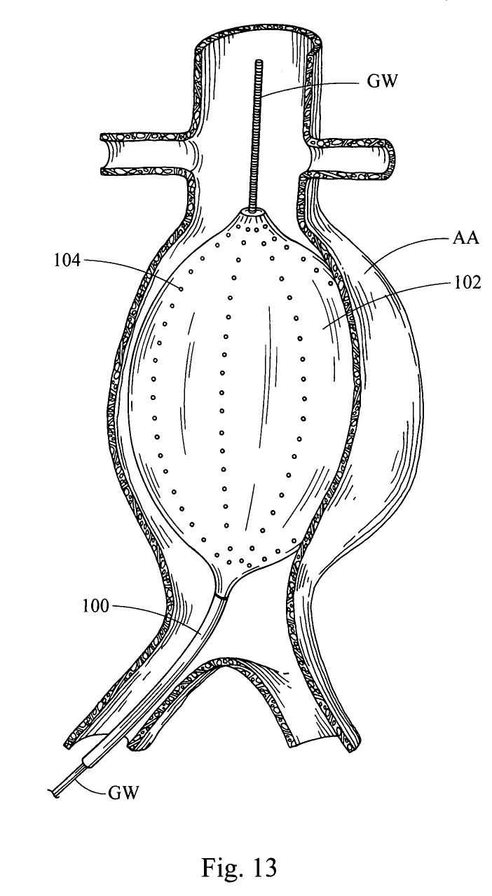

In other embodiment, referring now to FIG. 13, the cathepsin inhibitor or cathepsin inhibitor carrying solution (e.g., polymer carrier material) may be delivered by a catheter 100 having a perforated balloon 102 at its distal end. The catheter may be introduced over a guidewire to the target site, which is illustrated as an aortic aneurysm AA. The cathepsin inhibitor may be introduced to inflate the balloon and, once a sufficient pressure is relieved, to pass outwardly through the perforations 104 formed in a pre-selected pattern over the balloon surface. Usually, the perforations will be evenly distributed over the surface of the balloon, but there may be instances when asymmetric release is desired for some reason. While the cathepsin inhibitor or cathepsin inhibitor-carrying solution will usually be the balloon inflation medium, it will also be possible to separately inflate the balloon and to deliver cathepsin inhibitors through isolated lumen(s) formed in the balloon structure and/or catheter body.

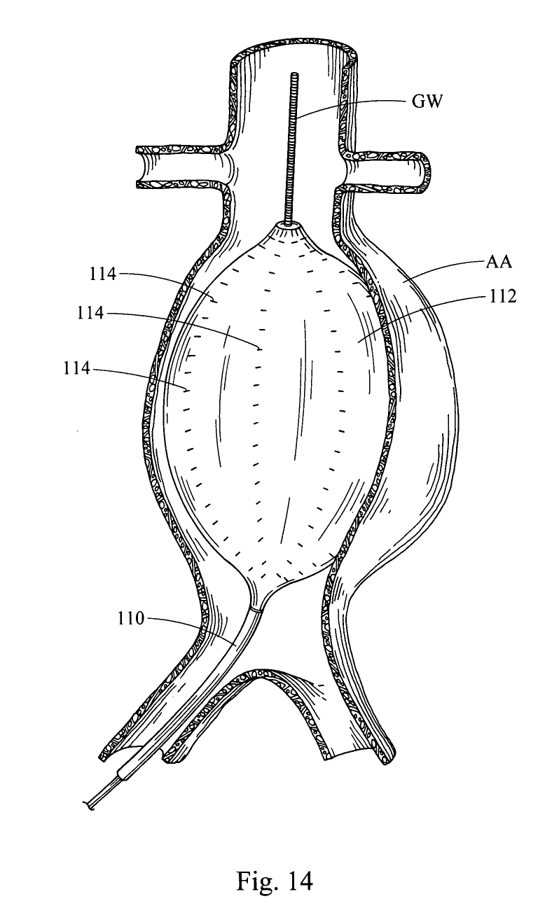

Referring now to FIG. 14, a catheter 110 which is also introducable over a guidewire GW is positioned to treat an aortic aneurysm AA. The catheter has an inflatable balloon 112 at its distal end, and the balloon has a plurality of microneedles 114 formed over its surface. The catheter may be operated to deliver the cathepsin inhibitor medium in manner analogous to that described with respect to FIG. 13, i.e., the medium may be used to inflate the balloon and, once a threshold pressure has been reached, be delivered through the microneedles which have penetrated into the aneurysmal wall. Alternatively, separate delivery means may be provided within the catheter in the balloon for delivering the medium through the needles 114 at a different pressure.

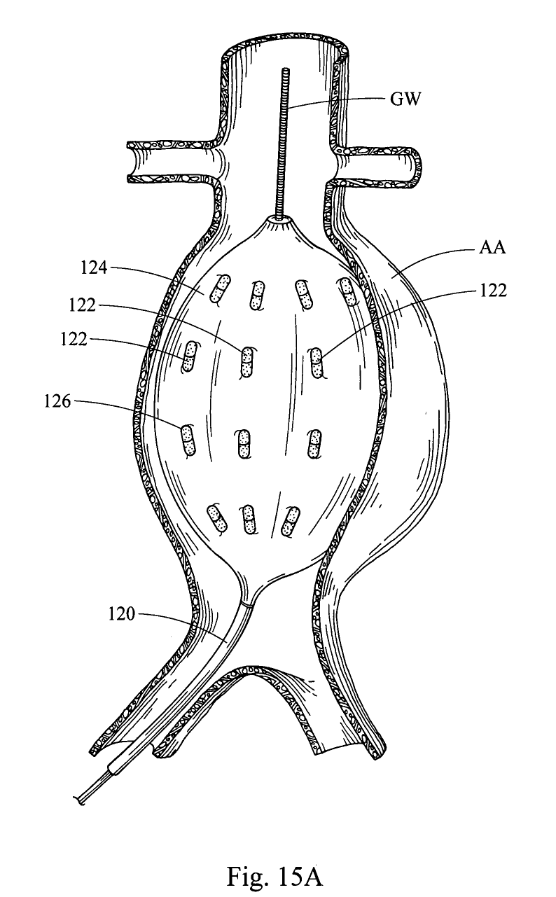

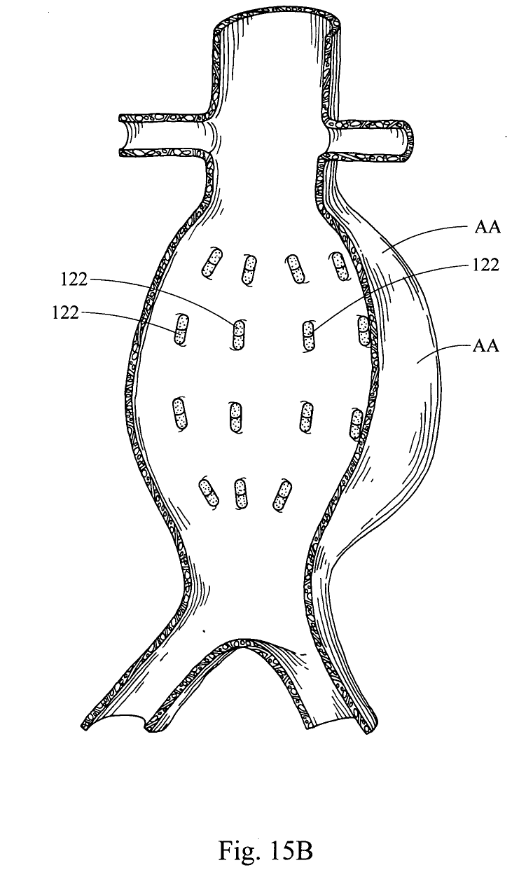

Referring now to FIG. 15, a catheter 120 may be utilized to deliver a plurality of delivery capsules, including cathepsin inhibitor 122 which are initially disposed over an exterior surface of an inflatable balloon 124, as illustrated in FIG. 15a. The catheter 120 may be positioned over a guidewire GW at the aortic aneurysm AA, as described previously. By inflating the balloon 124, the cathepsin inhibitor capsules 122 are implanted into the interior wall of the aneurysm AA, as illustrated in FIG. 15b. Catheter 120 may then be removed, leaving the inhibitor capsules 122 in place. The inhibitor capsules may be any of a variety of conventional controlled drug delivery structures intended to release the desired drug into the aneurysmal wall over time at a controlled rate. Optionally, the capsules may comprise hooks 126 (FIG. 15a) or other similar anchors for holding the capsules in the wall.

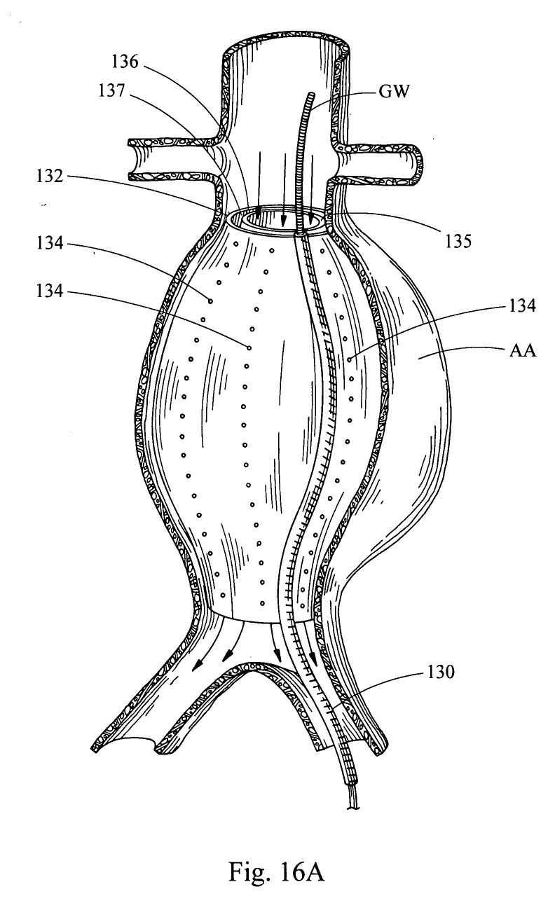

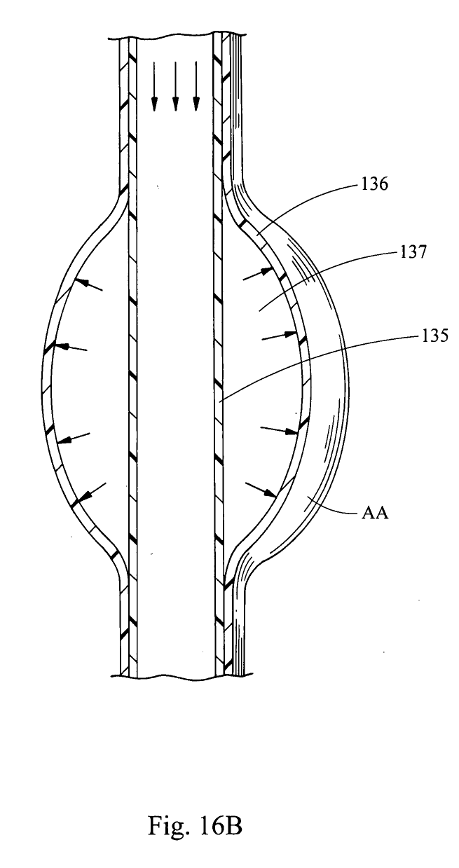

Referring now to FIGS. 16A and B, a catheter 130 includes a torroidal (i.e. coaxial, double) balloon 132 at its distal end. The catheter may be introduced over a guidewire GW to the aortic aneurysm AA generally as described previously. A balloon includes an inner channel for blood flow, inner 135 and outer 136 balloon, wherein the space 137 between the inner and outer balloon is filled with a cathepsin inhibitor or a polymer carrier material, including a cathepsin inhibitor. In this instance, the carrier material, including the cathepsin inhibitor may be used to inflate the balloon and conform to the shape of aneurysm. The polymer carrier material, including a cathepsin inhibitor may then be delivered through perforations 134 formed in the torroidal balloon 132. Preferably, the polymer carrier material releases the cathepsin inhibitor over a period of time. Alternatively the material may be delivered through separate, isolated delivery lumens in the balloon and the catheter. As the material is graduated or eluted through the perforations, the balloon may retract and pull the aneurysm inward, thereby shrinking it. An advantage of the torroidal balloon structure is that it allows blood flow through its center during balloon deployment and cathepsin inhibitor release.

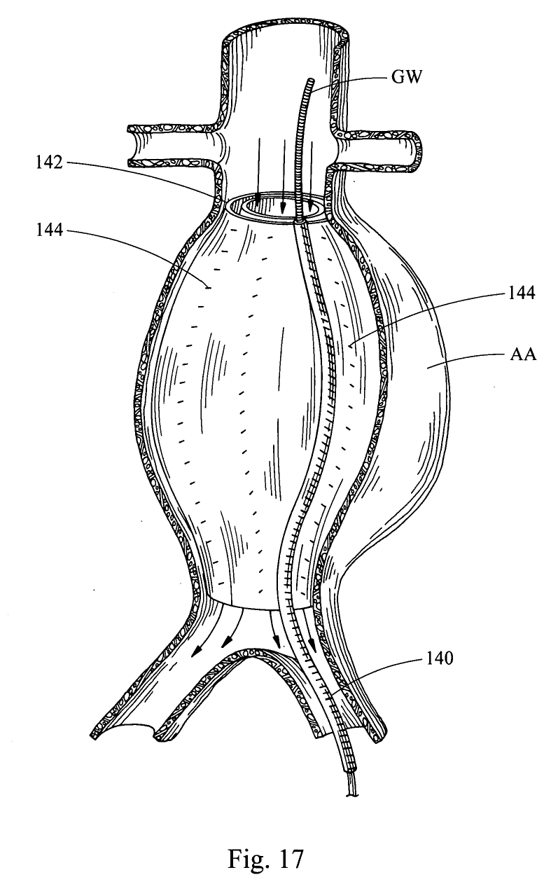

Referring now to FIG. 17, a catheter 140 includes a torroidal balloon 142 similar to that described in connection with catheter 130 above. Instead of perfusion holes, however, catheter 140 has microneedles 146 formed over its exterior surface. Catheter 140 is also intended for introducing to an aortic aneurysm AA over a guidewire GW, as previously discussed, and permits blood perfusion through the axial opening in the balloon 142. As with previous embodiments, the cathepsin inhibitor or cathepsin inhibitor carrying solution to be delivered may also serve as the inflation medium for the balloon 142. Alternatively, the cathepsin inhibitor may be delivered to the microneedles 144 via isolated lumens formed within the balloon and/or catheter.

The cathepsin inhibitor can also be placed on the balloon in a form of microencapsulated spheres, which are denoted by the reference numeral 12 and are disposed on the exterior of or extruded within the wall of a balloon 14 associated with a balloon catheter 16. The balloon catheter 16 and balloon 14 are conventional and well known in the art. The balloon catheter 16 is surgically or percutaneously inserted into an artery of the patient. The balloon catheter 16 may be coupled to an external shuttle gas source (not shown) to inflate and deflate the balloon 14.

Other examples of suitable balloons using microencapsulated spheres were previously described in U.S. Pat. No. 6,129,705. For example, with reference to FIG. 19, the balloon 171 includes an outer peripheral surface 172, on which a plurality of microencapsulated spheres 173 are impregnated in a coating material 174, which may be, but is not limited to, a hydrophilic material. The microencapsulated spheres 173 contain one or more cathepsin inhibitors, and are immersed in the coating 174. In an alternative embodiment, the microencapsulated spheres may be extruded in the wall of the balloon during the manufacturing process. The microencapsulated spheres 173 can be made from a biologically inert material, which may be a polymeric material, but is not limited to a polymeric material, and are sized (on the order of 5 microns, but not limited to such size) and configured to rupture upon application of a predetermined pressure caused by inflating the balloon 171. The microencapsulated spheres 173 are fabricated with a quantity of medicament in accordance with known techniques. These are described, for example, in articles entitled Intelligent Gels, Toyoichi Tanaka, Chemical & Engineering News, Page 26, Jun. 9, 1997, and Double Wall Microspheres—Advanced Drug Delivery, R & D, Page 64, March 1994. The technology described in the R & D Article has been licensed by Alkermes, of Cambridge Mass. The density of microencapsulated spheres 173 in the coating 174 is a function of the size of the spheres, balloon surface area and desired quantity of cathepsin inhibitor to be administered. The coating typically comprises a hydrophilic material, although other materials may be employed within the scope of the invention, and is on the order of about 0.127 μm thickness, but is not limited to such size.

Once the microspheres 173 become embedded in an artery wall 175 when an initial pressure is communicated to the balloon 171 as depicted in FIG. 19, and to thereafter rupture upon further inflation of the balloon 171 to cause the cathepsin inhibitor to be administered to the patient. The amount of pressure required is a function of the balloon geometry and material, as well as the configuration of the microencapsulated spheres 173. The arrangement described above allows for the delivery and release of the cathepsin inhibitor to a specific area, without undesirable occlusion of blood flow or dilution of the cathepsin inhibitor. It may also reduce the amount of time required for the balloon 171 to remain inflated within the artery. The microencapsulated sphere contents may be infused directly into the artery wall, and consequently the delivery is more effective.

In one embodiment, a photodynamic therapy (PDT) balloon catheter may be used when a cathepsin inhibitor is formulated to be taken up at the treatment site (e.g., bond with the elastin or other constituents of the wall), then infrared, UV or visible light (of wavelength of 200 nm up to 1200 nm) may be used to activate the drug. PDT balloon catheters were previously described in U.S. Pat. Nos. 5,797,868, 5,709,653, and 5,728,068. Two methods for photodynamic therapy (PDT) treatment of blood vessels including use of a balloon are disclosed in the Narciso, Jr. U.S. Pat. Nos. 5,169,395 and 5,298,018. The elastin-based biomaterials that may be used to for photodynamic therapy were described in U.S. Pat. No. 6,372,228.

In yet another embodiment, cathepsin inhibitors may be impregnated into a film material, which may be delivered via balloon.

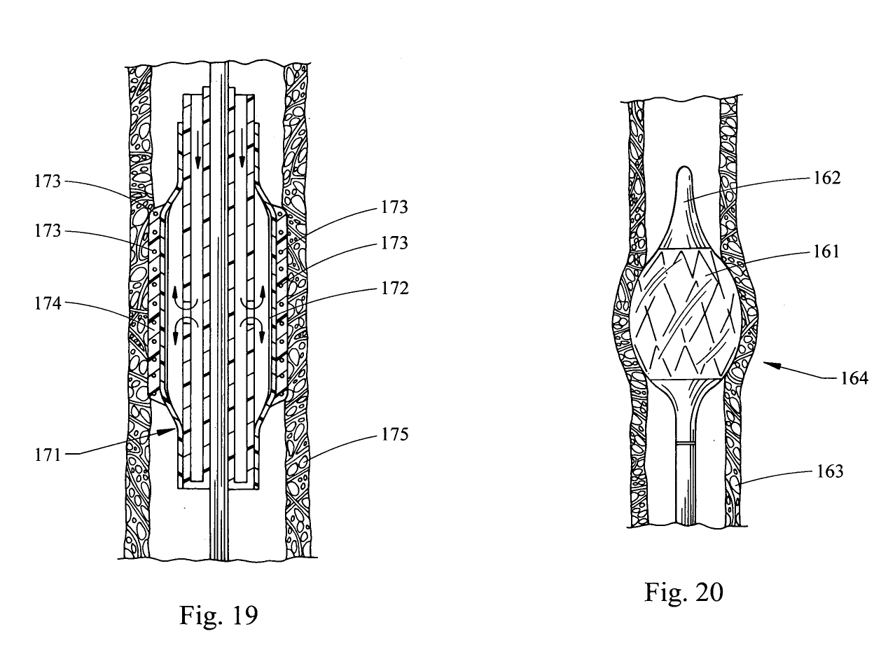

In one embodiment, a cathepsin-loaded film can be pre-mounted upon a deflated balloon catheter. Referring to FIG. 20, the balloon catheter can be maneuvered into the desired arterial or venous location 164 using standard techniques. The balloon 162 can then be inflated, compressing the stent (film material 161) against the vessel wall 163 and then the balloon can be deflated and removed leaving the cathepsin inhibitor-leaded film in place. A protective sleeve (e.g., of plastic) can be used to protect the stent during its passage to the vessel and then withdrawn once the film is in the desired location.

Similarly to stents and stent grafts, the cathepsin inhibitor is preferably released in a controlled manner over an extended time frame (e.g., months) using at least one of several well-known techniques involving polymer carriers or layers to control elution, which were described above.

D. Baskets

In one embodiment of this invention, the endoluminal device of this invention comprises a drug release system that releases a cathepsin inhibitor at predetermined location within a lumen of a patient, wherein the drug release system may be a structure such as catheter with a basket.

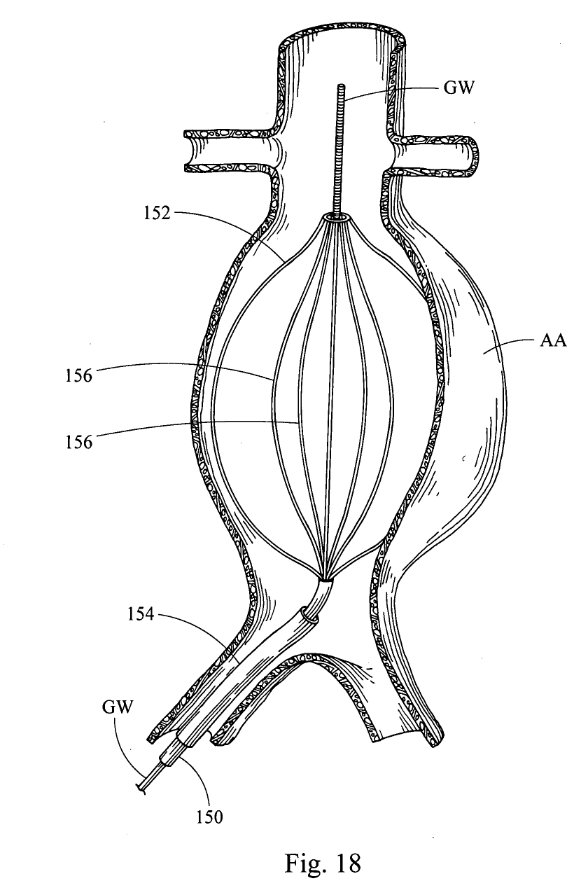

Referring now to FIG. 18, cathepsin inhibitors may be delivered using a catheter 150 introducable over a guidewire GW. The catheter 150 carries an expansible cage (basket) 152 at its distal end. As illustrated, the cage is constructed from elastic struts or tubes, typically formed from a shape memory alloy, such as a nickel-titanium alloy. Typically, the elastic struts will be coated with drugs, and the cage may be released and implanted in the manner of a stent. Alternatively, the tubes include lumens for infusing the cathepsin inhibitor to be delivered. The cage may be selectively expanded and collapsed by withdrawing or extending the cage from a cover sheath 154. The cathepsin inhibitors may be delivered through the longitudinal elements 156 of the sheath through holes, microneedles, or other release (not shown).

VI. Treatment

In one embodiment, the invention is directed to a method for treating endovascular disease, including aneurysms, such as abdominal aortic aneurysm. The method comprises placing an endoluminal medical device comprising a drug release system that releases a cathepsin inhibitor to a location near the aneurysm.

Preferably, the device includes a polymer layer and/or any optional polymer layers described above configured to provide a controlled release of the cathepsin inhibitor. The device may be advanced into the patient using conventional techniques such as over a guiding catheter with an advancing catheter or element.

The remaining details of the method of medical treatment are the same as those disclosed with respect to the method of making the device 10 of the present invention; for the sake of brevity, they need not be repeated here.

VII. Combination Therapy

In one embodiment, the invention provides a medical device comprising one or more cathepsin inhibitor compounds and one or more other bioactive agents. In one embodiment, therapeutically effective amounts of the cathepsin inhibitor compound and bioactive agents are provided. Examples of suitable cathepsin inhibitor compounds were described above.

Other bioactive agents may be incorporated with the medical device using the methods which were described above in connection with incorporating the cathepsin inhibitor compounds with the medical device of this invention.

Other bioactive agents that may be incorporated with the medical device of this invention include MMPs inhibitors, including endogenous inhibitors, such as tissue inhibitors of MMPs (TIMPs) and α-macroglobulins, and synthetic inhibitors, such as chelating agents (e.g., EDTA and 1,10-phenanthroline), peptides, antibodies, and the like. Agents that would enhance function of TIMPs may also be used.

Any suitable tetracycline, including tetracycline per se, or tetracycline-derivative compounds, such as for example, doxycycline hydrate, doxycycline aureomycin and chloromycin may be included. Preferred tetracycline compounds include CMTs (CMT that lack the dimethylamino group at position 4 of the ring structure of tetracycline, including 4-dedimethylaminotetracycline (CMT-1), 4-dedimethylamino-5-oxytetracycline, 4-dedimethylamino-7-chlorotetracycline (CMT-4), 4-hydroxy-4-dedimethylaminotetracycline (CMT-6), 5a,6-anhydro-4-hydroxy-4-dedimethylaminotetracycline, 6-demethyl-6-deoxy-4-dedimethylaminotetracycline (CMT-3; COL-3), 4-dedimethylamino-12a-deoxytetracycline (CMT-7), and 6-α-deoxy-5-hydroxy-4-dedimethylaminotetracycline (CMT-8); tetracyclines modified at the 2 carbon position to produce a nitrile, e.g., tetracyclinonitrile; 6-α-benzylthiomethylenetetracycline, the mono-N-alkylated amide of tetracycline, 6-fluoro-6-demethyltetracycline, and 11α-chlorotetracycline).

In another embodiment beta blockers may be included. Beta blockers include acebutolol, atenolol, betaxolol, bisoprolol, carteolol, carvedilol, esmolol, labetolol, metoprolol, nadolol, penbutolol, pindolol, propranolol, and timolol.

Other bioactive agents useful in embodiments of this invention include cyclooxygenase-2 (COX-2) inhibitors; angiotensin-converting enzyme (ACE) inhibitors; glucocorticoids; nitric acid synthase (NOS) inhibitors; other anti-inflammatories; anti-oxidants; and cellular adhesion molecules (CAMs).

COX-2 inhibitors include Celecoxib, Rofecoxib, Valdecoxib, Etoricoxib, Parecoxib, all of which are available in pharmacological preparations. Additionally, COX-2 inhibition has been demonstrated from herbs, such as green tea, ginger, turmeric, chamomile, Chinese gold-thread, barberry, baikal skullcap, Japanese knotweed, rosemary, hops, feverfew, and oregano; and other agents, such as piroxican, mefenamic acid, meloxican, nimesulide, diclofenac, MF-tricyclide, raldecoxide, nambumetone, naproxen, herbimycin-A, and diaryl hydroxyfuranones.

NSAIDs that may be used in embodiments according to the present invention include ketoralac tromethamine (Toradol), indomethacin, ketorolac, ibuprofen and aspirin among others. Additionally, steroidal based anti-inflammatories, such as methylprednisolone, dexamethasone or sulfasalazine may be provided. Other suitable anti-inflammatory agents include cyclosporine A and azathioprine.

Another type of suitable bioactive agents are anti-oxidants, such as curcumin, vitamins, and vitamin constituents, such as α-tocopherol and β-carotene.

Yet other bioactive agents include ACE inhibitors, such as captopril, enalapril, losartan and lisinopril and the active forms of several ACE inhibitor prodrugs on the market.

Yet another group of bioactive agents include elastin-stabilizing compounds, such as tannic acid. Exemplary elastin-stabilizing compounds as well as medical devices including elastin-stabilizing compounds were previously described in U.S. Provisional Pat. Application Ser. No. 60/799,608, filed May 10, 2006, which is incorporated herein by reference in its entirety.

Other bioactive agents, such as the NOS inhibitors, including aminoguanidine are also useful in combination with the cathepsin inhibitor compounds of the present invention.

The invention also provides medical device coatings comprising the cathepsin inhibitor compounds in combination with one or more bioactive agents described in U.S. Pat. No. 5,834,449; U.S. Publication Nos. 2005/0266043 A1, published on Dec. 1, 2005, and 2006/0004441 A1, published on Jan. 5, 2006, which are incorporated herein by reference.

In addition to the embodiments described above, the invention includes combinations of the preferred embodiments discussed above, and variations of all embodiments.

VIII. Kits

In addition, the invention concerns a kit comprising an endoluminal medical device, comprising a drug release system that releases cathepsin inhibitor at a predetermined location within a lumen of a patient. The kit may also include a delivery system for inserting the endoluminal device into a lumen of a body, wherein the endoluminal device is the endoluminal device as disclosed above. In an aspect, the invention relates to these kits wherein the delivery system is an intraluminal catheter.

The invention also relates to these kits where the cathepsin inhibitor is contained within the drug release system. The invention also relates to these kits wherein the cathepsin inhibitor is present in an amount effective to inhibit cathepsins once the device is deployed. The invention also relates to these kits where the drug release system is a stent, covered stent, balloon, torroidal balloon, or basket.

The kit may further include instructional materials.

A consensus document has been assembled by clinical, academic, and industrial investigators engaged in preclinical interventional device evaluation to set forth standards for evaluating devices, including drug-eluting stents such as those contemplated by the present invention. See “Drug-Eluting Stents in Preclinical Studies—Recommended Evaluation From a Consensus Group” by Schwartz and Edelman (available at http://www.circulationaha.org) (incorporated herein by reference).

In view of the disclosure above, it is clear that the described embodiments can provide an endoluminal medical device which achieves precise control over the release of cathepsin inhibitor or inhibitors contained in the device. The cathepsin inhibitor can be supplied to any of a wide variety of locations within a patient during or after the performance of a medical procedure, but are especially useful for preventing further degradation of host connective tissue, which can result in dialation of vessel and aneurysm, by the delivery cathepsin inhibitors to the region. They can permit the release rate of a cathepsin inhibitor to be carefully controlled over both the short and long terms. Most importantly, any degradation of the cathepsin inhibitor which might otherwise occur may be avoided by application of, for example, a polymer coating.

The device and method are useful in the performance of vascular surgical procedures, and therefore find applicability in human and veterinary medicine.

The other details of the construction or composition of the various elements of the disclosed embodiments of the present invention are not believed to be critical to the achievement of the advantages of the device and method, so long as the elements possess the strength or flexibility needed for them to perform as disclosed. The selection of these and other details of construction are believed to be well within the ability of one of ordinary skills in this area, in view of the present disclosure.

It is to be understood that the above-described devices are merely illustrative embodiments of the principles taught herein, and that other devices and methods for using them may be devised by those skilled in the art, without departing from the scope of the claims. It is also to be understood that the invention is directed to embodiments both comprising and consisting of the disclosed parts.

### EXAMPLES

### Example 1

### Testing Compounds as Cathepsin Inhibitors

The cathepsin inhibitory effects of the compound of the invention can be determined in vitro by measuring the inhibition of, e.g., recombinant human cathepsins B, K, L and S. The buffer for use in the cathepsin B, L and S assays is a 0.1 M pH 5.8 phosphate buffer containing EDTA (1.33 mM), DTT (2.7 mM) and Brij (0.03%). The in vitro assays are carried out as follows:

(a) For cathepsin B:

To a microtiter well is added 100 uL of a 20 uM solution of inhibitor in assay buffer followed by 50 uL of a 6.4 mM solution of Z-Arg-Arg-AMC substrate (Peptides International) in assay buffer. After mixing, 50 uL of a 0.544 nM solution of recombinant human cathepsin B in assay buffer is added to the well, yielding a final inhibitor concentration of 10 uM. Enzyme activity is determined by measuring fluorescence of the liberated aminomethylcoumarin at 440 nM using 380 nM excitation, at 20 minutes. % Enzyme inhibition is determined by comparison of this activity to that of a solution containing no inhibitor. Compounds are subsequently subjected to a dose response curve analysis to determine IC₅₀ values.

(b) For Cathepsin K:

The assay is performed in 96 well microtiter plates at ambient temperature using recombinant human cathepsin K. Inhibition of cathepsin K is assayed at a constant enzyme (0.16 nM) and substrate concentration (54 mM Z-Phe-Arg-MCA—Peptide Institute Inc. Osaka, Japan) in 100 mM sodium phosphate buffer, pH 7.0, containing 2 mM dithiothreitol, 20 mM Tween 80 and 1 mM EDTA. Cathepsin K is pre-incubated with the inhibitors for 30 min, and the reaction is initiated by the addition of substrate. After 30 min incubation the reaction is stopped by the addition of E-64 (2 mM), and fluorescence intensity is read on a multi-well plate reader at excitation and emission wavelengths of 360 and 460 nm, respectively.

(c) For Cathepsin L:

Recombinant human cathepsin L is activated prior to use in this assay: To 500 uL of a 510 nM solution of cathepsin L in a 50 mM pH 5.0 acetate buffer containing 1 mM EDTA, 3 mM DTT and 150 mM NaCl is added 10 uL of a 625 uM solution of dextran sulfate (ave. mw=8000), and the resulting solution is incubated on ice for 30 min. 4 uL of this solution is then diluted into 46 uL assay buffer, yielding a 40 nM enzyme solution.

To perform the assay, 100 uL of a 20 uM solution of inhibitor in assay buffer is added to a microtiter well. 50 uL of a 20 uM solution of Z-Phe-Arg-AMC (Peptides International) is then added. After mixing, 50 uL of the activated 40 nM solution of recombinant human cathepsin L in assay buffer is then added to the well, yielding a final inhibitor concentration of 10 uM. Enzyme activity is determined by measuring fluorescence of the liberated aminomethylcoumarin at 440 nM using 380 nM excitation of 20 minutes. % Enzyme inhibition is determined by comparison of this activity to that of a solution containing no inhibitor. Compounds are subsequently subjected to a dose response curve analysis to determine IC₅₀ values.

(d) For Cathepsin S:

To a microtiter well is added 100 uL of a 20 uM solution of inhibitor is assay buffer. 50 uL of a 700 uM solution of Z-Val-Val-Arg-AMC substrate (Peptides International) is then added. After mixing, 50 uL of a 5.2 nM solution of recombinant human cathepsin S in assay buffer is then added to the well, yielding a final inhibitor concentration of 10 uM. Enzyme activity is determined by measuring fluorescence of the liberated aminomethylcoumarin at 440 nM using 380 nM excitation at 200 minutes. % Enzyme inhibition is determined by comparison of this activity to that of a solution containing no inhibitor. Compounds are subsequently subjected to a dose response curve analysis to determine IC₅₀ values.

### Additional Embodiments

In one embodiment, the invention is an endoluminal medical device including a drug release system operable to release a cathepsin inhibitor at a predetermined location within a lumen of a patient. The cathepsin inhibitor may be selected from the group consisting of cysteine proteinase inhibitor, aspartic proteinase inhibitors, and serine proteinase inhibitors. The cathepsin inhibitor may be selected from inhibitors selected from the group consisting of inhibitors of cathepsin B, inhibitors of cathepsin C, inhibitors of cathepsin H, inhibitors of cathepsin L, inhibitors of cathepsin S, inhibitors of cathepsin S, inhibitors of cathepsin K, inhibitors of cathepsin O, inhibitors of cathepsin D, inhibitors of cathepsin E, inhibitors of cathepsin G, and inhibitors of cathepsin A. The cathepsin inhibitor may be selected from the group consisting of compounds CP-1, CP-2, CP-3 from Aspergillus sp.; epoxysuccinamide derivative; peptide derivative; epoxysuccinamide derivative; thiomethylene-containing aldehyde; Monobactam derivative; peptidic oxadiazole and oxathiazole derivatives; 3,4-disubstituted azetidin-2-one derivatives; 4-substituted-3-(2-amino-2-cycloalkylmethylacetamido)azetidin-2-one derivatives; β-lactam penam and cepham derivatives; O-benzoylhydroxylaminoe dipeptides; piperidylketocarboxylic acids; benzamidoaldehyde; ketobenzamide; heterocyclic substituted benzamide; substituted oxodiazole derivatives; ketoamide derivatives; Quinolone-containing ketoamide; dipeptide nitrile derivatives; thiadiazole derivatives; substituted benzamides; N-carbonylalkyl-benzamide; heterocyclically-substituted amide derivatives; N-cyanomethyl-amide derivative; amide derivatives; 3-acetamidoazetidin-2-one derivatives; dipeptide derivatives; cyclic amide hypercalcaemia and dipeptide derivative; substituted pyrrolidin-2-one derivative; N-aminoalkyl-N-hydrazine derivatives; diacyl carbohydrazine compounds; thiazole guanidine derivatives; morpholinoethoxybenzofuran compounds; butyl amide derivatives; sulfonamide and carboxamide derivatives; modulated amyloid precursor protein and tau protein; hydroxypropylamide peptidomimetics; hydroxystatine amide hydroxyphosphonate peptidomimetics; hydroxyamino acid amide derivatives; peptoid compounds; heteroaryl amidines methylamidines and guanidines; 1,2,5-thiadiazolidin-3-one 1,1-dioxide derivatives; transhexahydro-pyrrolo[3,2-b]pyrrolone derivatives; pyrolopyrrolidine derivatives; furopyrrolidine derivatives; and anthraquinone derivatives; and mixtures thereof. The cysteine proteinase inhibitor may be an endogenous cathepsin inhibitor. The cysteine proteinase inhibitor may be an exogenous cysteine proteinase inhibitor. The exogenous cysteine proteinase inhibitor may be a small peptide derivative or a beta phosphonic acid. The cathepsin inhibitor may be a dipeptide nitrile. The drug release system may comprise a stent. The stent may be a self-expanding stent or a balloon expandable stent. The drug release system comprises a tubular graft material supported by the stent. The graft material may comprise an extracellular matrix material. The device may comprise a delivery system for delivering the device and wherein the drug release system may be integrated with the delivery system. The delivery system may comprise a balloon and wherein the drug release system may be integrated with the balloon. The balloon may include one or more perforations configured to release the cathepsin inhibitor. The cathepsin inhibitor may be carried on an outer surface of the balloon. The balloon may be a torroidal balloon. The balloon may be a photodynamic therapy balloon. The drug release system may comprise an expandable wire basket. The device, as described above may comprise a polymer layer to provide a controlled release of the cathepsin inhibitor at the predetermined location. The device, as described above, wherein the device may be configured for treatment of an aneurysm. The aneurysm may be an abdominal aortic aneurysm. The device, as described above, wherein a plurality of cathepsin inhibitor compounds may be incorporated in multiple coating layers. An endoluminal medical device as described above, wherein the device may be for treating an aneurysm, such as abdominal aortic aneurysm, and the drug release system may be operable to release cathepsin inhibitor at a location near the aneurysm.

In another embodiment, the invention is a method of treating an aneurysm, the method comprising delivering a cathepsin inhibitor releasing device to a location near the aneurysm. The device may be an endoluminal device comprising a drug release system that releases the cathepsin inhibitor. The endoluminal device may be a stent graft for treating an aortic aneurysm. In the method, the cathepsin inhibitor may be selected from the group consisting of cysteine proteinase inhibitor, aspartic proteinase inhibitors, and serine proteinase inhibitors. In the method, the cathepsin inhibitor may be selected from inhibitors selected from the group consisting of inhibitors of cathepsin B, inhibitors of cathepsin C, inhibitors of cathepsin H, inhibitors of cathepsin L, inhibitors of cathepsin S, inhibitors of cathepsin S, inhibitors of cathepsin K, inhibitors of cathepsin O, inhibitors of cathepsin D, inhibitors of cathepsin E, inhibitors of cathepsin G, and inhibitors of cathepsin A. In the method, the cathepsin inhibitor may be selected from the group consisting of compounds CP-1, CP-2, CP-3 from Aspergillus sp.; epoxysuccinamide derivative; peptide derivative; epoxysuccinamide derivative; thiomethylene-containing aldehyde; Monobactam derivative; peptidic oxadiazole and oxathiazole derivatives; 3,4-disubstituted azetidin-2-one derivatives; 4-substituted-3-(2-amino-2-cycloalkylmethylacetamido)azetidin-2-one derivatives; β-lactam penam and cepham derivatives; O-benzoylhydroxylaminoe dipeptides; piperidylketocarboxylic acids; benzamidoaldehyde; ketobenzamide; heterocyclic substituted benzamide; substituted oxodiazole derivatives; ketoamide derivatives; Quinolone-containing ketoamide; dipeptide nitrile derivatives; thiadiazole derivatives; substituted benzamides; N-carbonylalkyl-benzamide; heterocyclically-substituted amide derivatives; N-cyanomethyl-amide derivative; amide derivatives; 3-acetamidoazetidin-2-one derivatives; dipeptide derivatives; cyclic amide hypercalcaemia and dipeptide derivative; substituted pyrrolidin-2-one derivative; N-aminoalkyl-N-hydrazine derivatives; diacyl carbohydrazine compounds; thiazole guanidine derivatives; morpholinoethoxybenzofuran compounds; butyl amide derivatives; sulfonamide and carboxamide derivatives; modulated amyloid precursor protein and tau protein; hydroxypropylamide peptidomimetics; hydroxystatine amide hydroxyphosphonate peptidomimetics; hydroxyamino acid amide derivatives; peptoid compounds; heteroaryl amidines methylamidines and guanidines; 1,2,5-thiadiazolidin-3-one 1,1-dioxide derivatives; transhexahydro-pyrrolo[3,2-b]pyrrolone derivatives; pyrolopyrrolidine derivatives; furopyrrolidine derivatives; and anthraquinone derivatives; and mixtures thereof. In the method, the cysteine proteinase inhibitor may be an endogenous cathepsin inhibitor. In the method, the cysteine proteinase inhibitor may be an exogenous cysteine proteinase inhibitor. In the method, the exogenous cysteine proteinase inhibitor may be a small peptide derivative or beta phosphonic acid. In the method, the cathepsin inhibitor may be a dipeptide nitrile.

In yet another embodiment, the present invention is a cathepsin inhibitor for use in therapy. The cathepsin inhibitor may be for use in treating an aneurysm. The cathepsin inhibitor may be a cysteine proteinase inhibitor, an aspartic proteinase inhibitor or a serine proteinase inhibitor. The cathepsin inhibitor may be selected from inhibitors selected from the group consisting of inhibitors of cathepsin B, inhibitors of cathepsin C, inhibitors of cathepsin H, inhibitors of cathepsin L, inhibitors of cathepsin S, inhibitors of cathepsin S, inhibitors of cathepsin K, inhibitors of cathepsin O, inhibitors of cathepsin D, inhibitors of cathepsin E, inhibitors of cathepsin G, and inhibitors of cathepsin A. The inhibitor may be a cysteine proteinase inhibitor and wherein the cysteine proteinase inhibitor may be an endogenous cysteine proteinase inhibitor. The cathepsin inhibitor may be a cysteine proteinase inhibitor and wherein the cysteine proteinase inhibitor may be an exogenous cysteine proteinase inhibitor. The exogenous cysteine proteinase inhibitor may be a small peptide derivative or a beta phosphonic acid. The cathepsin inhibitor may be CP-1, CP-2, CP-3 from Aspergillus sp.; epoxysuccinamide derivative; peptide derivative; epoxysuccinamide derivative; thiomethylene-containing aldehyde; Monobactam derivative; peptidic oxadiazole and oxathiazole derivatives; 3,4-disubstituted azetidin-2-one derivatives; 4-substituted-3-(2-amino-2-cycloalkylmethylacetamido)azetidin-2-one derivatives; β-lactam penam and cepham derivatives; O-benzoylhydroxylaminoe dipeptides; piperidylketocarboxylic acids; benzamidoaldehyde; ketobenzamide; heterocyclic substituted benzamide; substituted oxodiazole derivatives; ketoamide derivatives; Quinolone-containing ketoamide; dipeptide nitrile derivatives; thiadiazole derivatives; substituted benzamides; N-carbonylalkyl-benzamide; heterocyclically-substituted amide derivatives; N-cyanomethyl-amide derivative; amide derivatives; 3-acetamidoazetidin-2-one derivatives; dipeptide derivatives; cyclic amide hypercalcaemia and dipeptide derivative; substituted pyrrolidin-2-one derivative; N-aminoalkyl-N-hydrazine derivatives; diacyl carbohydrazine compounds; thiazole guanidine derivatives; morpholinoethoxybenzofuran compounds; butyl amide derivatives; sulfonamide and carboxamide derivatives; modulated amyloid precursor protein and tau protein; hydroxypropylamide peptidomimetics; hydroxystatine amide hydroxyphosphonate peptidomimetics; hydroxyamino acid amide derivatives; peptoid compounds; heteroaryl amidines methylamidines and guanidines; 1,2,5-thiadiazolidin-3-one 1,1-dioxide derivatives; transhexahydro-pyrrolo[3,2-b]pyrrolone derivatives; pyrolopyrrolidine derivatives; furopyrrolidine derivatives; or anthraquinone derivatives; or mixtures thereof. The cathepsin inhibitor may be a dipeptide nitrile.

In a further embodiment, the invention is directed to use of a cathepsin inhibitor in the manufacture of an endoluminal medical device for treating an aneurysm. The cathepsin inhibitor may be a cysteine proteinase inhibitor, an aspartic proteinase inhibitor or a serine proteinase inhibitor. The cathepsin inhibitor may be a cysteine proteinase inhibitor and wherein the cysteine proteinase inhibitor may be an endogenous cysteine proteinase inhibitor. The inhibitor may be a cysteine proteinase inhibitor and wherein the cysteine proteinase inhibitor may be an exogenous cysteine proteinase inhibitor. The exogenous cysteine proteinase inhibitor may be a small peptide derivative or a beta phosphonic acid. The cathepsin inhibitor is selected from inhibitors selected from the group consisting of inhibitors of cathepsin B, inhibitors of cathepsin C, inhibitors of cathepsin H, inhibitors of cathepsin L, inhibitors of cathepsin S, inhibitors of cathepsin S, inhibitors of cathepsin K, inhibitors of cathepsin O, inhibitors of cathepsin D, inhibitors of cathepsin E, inhibitors of cathepsin G, and inhibitors of cathepsin A. The cathepsin inhibitor may be CP-1, CP-2, CP-3 from Aspergillus sp.; epoxysuccinamide derivative; peptide derivative; epoxysuccinamide derivative; thiomethylene-containing aldehyde; Monobactam derivative; peptidic oxadiazole and oxathiazole derivatives; 3,4-disubstituted azetidin-2-one derivatives; 4-substituted-3-(2-amino-2-cycloalkylmethylacetamido)azetidin-2-one derivatives; β-lactam penam and cepham derivatives; O-benzoylhydroxylaminoe dipeptides; piperidylketocarboxylic acids; benzamidoaldehyde; ketobenzamide; heterocyclic substituted benzamide; substituted oxodiazole derivatives; ketoamide derivatives; Quinolone-containing ketoamide; dipeptide nitrile derivatives; thiadiazole derivatives; substituted benzamides; N-carbonylalkyl-benzamide; heterocyclically-substituted amide derivatives; N-cyanomethyl-amide derivative; amide derivatives; 3-acetamidoazetidin-2-one derivatives; dipeptide derivatives; cyclic amide hypercalcaemia and dipeptide derivative; substituted pyrrolidin-2-one derivative; N-aminoalkyl-N-hydrazine derivatives; diacyl carbohydrazine compounds; thiazole guanidine derivatives; morpholinoethoxybenzofuran compounds; butyl amide derivatives; sulfonamide and carboxamide derivatives; modulated amyloid precursor protein and tau protein; hydroxypropylamide peptidomimetics; hydroxystatine amide hydroxyphosphonate peptidomimetics; hydroxyamino acid amide derivatives; peptoid compounds; heteroaryl amidines methylamidines and guanidines; 1,2,5-thiadiazolidin-3-one 1,1-dioxide derivatives; transhexahydro-pyrrolo[3,2-b]pyrrolone derivatives; pyrolopyrrolidine derivatives; furopyrrolidine derivatives; or anthraquinone derivatives; or mixtures thereof. The cathepsin inhibitor may be a dipeptide nitrile.

It is therefore intended that the foregoing detailed description be regarded as illustrative rather than limiting.

1. An endoluminal medical device including a drug release system operable to release a cathepsin inhibitor at a predetermined location within a lumen of a patient.

2. The device of claim 1, wherein the cathepsin inhibitor is selected from the group consisting of cysteine proteinase inhibitor, aspartic proteinase inhibitors, and serine proteinase inhibitors.

3. The device of claim 1, wherein the cathepsin inhibitor is selected from inhibitors selected from the group consisting of inhibitors of cathepsin B, inhibitors of cathepsin C, inhibitors of cathepsin H, inhibitors of cathepsin L, inhibitors of cathepsin S, inhibitors of cathepsin S, inhibitors of cathepsin K, inhibitors of cathepsin O, inhibitors of cathepsin D, inhibitors of cathepsin E, inhibitors of cathepsin G, and inhibitors of cathepsin A.

4. The device of claim 1, wherein the drug release system comprises a stent.

5. The device of claim 4, wherein the stent is a self-expanding stent or a balloon expandable stent.

6. The device of claim 1, wherein the drug release system comprises a tubular graft material supported by the stent.

7. The device of claim 6, wherein the graft material comprises an extracellular matrix material.

8. The device of claim 1, wherein the device comprises a delivery system for delivering the device and wherein the drug release system is integrated with the delivery system.

9. The device of claim 1, comprising a polymer layer to provide a controlled release of the cathepsin inhibitor at the predetermined location.

10. The device of claim 1, wherein the device is configured for treatment of an aneurysm.

11. The device of claim 10, wherein the aneurysm is an abdominal aortic aneurysm.

12. The device of claim 1, wherein the device is configured for treatment of an aortic dissection.

13. The device of claim 1, wherein a plurality of cathepsin inhibitor compounds are incorporated in multiple coating layers.

14. The device of claim 1, wherein the device is for treating an aneurysm and the drug release system is operable to release cathepsin inhibitor at a location near the aneurysm.

15. The device of claim 14, wherein the aneurysm is an abdominal aortic aneurysm.

16. A method of treating an aneurysm, the method comprising delivering a cathepsin inhibitor releasing device to a location near the aneurysm.

17. The method of claim 16, wherein the device is an endoluminal device comprising a drug release system that releases the cathepsin inhibitor.

18. The method of claim 17, wherein the endoluminal device is a stent graft for treating an aortic aneurysm.

19. The method of claim 16, wherein the cathepsin inhibitor is selected from the group consisting of cysteine proteinase inhibitor, aspartic proteinase inhibitors, and serine proteinase inhibitors.

20. The method of claim 16, wherein the cathepsin inhibitor is selected from inhibitors selected from the group consisting of inhibitors of cathepsin B, inhibitors of cathepsin C, inhibitors of cathepsin H, inhibitors of cathepsin L, inhibitors of cathepsin S, inhibitors of cathepsin S, inhibitors of cathepsin K, inhibitors of cathepsin O, inhibitors of cathepsin D, inhibitors of cathepsin E, inhibitors of cathepsin G, and inhibitors of cathepsin A.

21. A cathepsin inhibitor for use in therapy.

22. The cathepsin inhibitor of claim 21, for use in treating an aneurysm.

23. Use of a cathepsin inhibitor in the manufacture of an endoluminal medical device for treating an aneurysm.
